(19) United States
(12) Patent Application Publication
(10) Pub. No.: US 2023/0185191 A1
(43) Pub. Date: Jun. 15, 2023

(54) COMPOUND, PRODUCTION METHOD THEREFOR, ACID GENERATOR, COMPOSITION, RESIST FILM, UNDERLAYER FILM, PATTERN FORMATION METHOD, AND OPTICAL COMPONENT

(71) Applicants: The School Corporation Kansai University, Osaka (JP); MITSUBISHI GAS CHEMICAL COMPANY, INC., Tokyo (JP)

(72) Inventors: Hiroto KUDO, Osaka (JP); Takashi SATO, Kanagawa (JP); Tadashi OMATSU, Kanagawa (JP); Masatoshi ECHIGO, Tokyo (JP)

(21) Appl. No.: 17/924,277

(22) PCT Filed: May 10, 2021
(86) PCT No.: PCT/JP2021/017656

§ 371 Date: Nov. 9, 2022

(30) Foreign Application Priority Data
May 11, 2020 (JP) 2020-083105
May 11, 2020 (JP) 2020-083107

(51) Int. Cl. G03F 7/004; G03F 7/11; C07C 381/12; C07C 309/06; C07D 311/78

(52) U.S. Cl. CPC G03F 7/0045; G03F 7/11; C07C 381/12; C07C 309/06; C07D 311/78

(57) ABSTRACT

To provide a compound which is high in sensitivity and which provides high resolution and high flatness. A compound represented by the following formula (P-0):

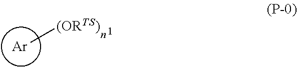

wherein Ar is a group having an aryl group having 6 to 60 carbon atoms, each OR^{TS} is independently a hydroxy group or a group having a specified ion moiety, and n¹ is an integer of 1 to 20, provided that at least one OR^{TS} is a group having a specified ion moiety.

### TECHNICAL FIELD

The present invention relates to a compound and a method for producing the compound, an acid generating agent, a composition, a resist film, an underlayer film, a method for forming a pattern, and an optical article.

### BACKGROUND ART

While fine processing lithography with photoresist materials has been performed in production of semiconductor devices, further refining by pattern rules has been recently demanded along with higher integration and higher speed of LSI (large-scale integrated circuit).

Conventional common resist materials have been polymer-based resist materials capable of forming amorphous thin films. Examples include polymer-based resist materials such as polymethyl methacrylate, polyhydroxystyrene or polyalkyl methacrylate having a dissociation reaction group. A substrate is coated with a solution of such a polymer-based resist material to thereby produce a resist thin film, and the resist thin film is irradiated with ultraviolet light, far ultraviolet light, electron beam, extreme ultraviolet light (Extreme UltraViolet: hereinafter, appropriately referred to as “EUV”). X-ray or the like to thereby form a line pattern of about 45 to 100 nm (see, for example, NPL 1).

However, such a polymer-based resist material has a high molecular weight of about 10,000 to 100,000, and also exhibits a broad molecular weight distribution. Thus, lithography with such a polymer-based resist material causes roughness on a fine pattern surface, making control of a pattern dimension difficult, to lead to a decrease in yield. Accordingly, there has been a limit on refining in lithography with a conventional polymer-based resist material. There have been proposed various low-molecular weight resist materials in order to produce a finer pattern.

For example, there has been proposed an alkaline development type negative radiation-sensitive composition with a low-molecular weight polynuclear polyphenol compound as a main component (see, for example, PTL 1 and PTL 2). There has also been proposed, as a candidate of a low-molecular weight resist material having high heat resistance, an alkaline development type negative radiation-sensitive composition with a low-molecular weight cyclic polyphenol compound as a main component (see, for example, PTL 3 and NPL 2). Furthermore, it is known that a polyphenol compound as a base compound of a resist material, while has a low molecular weight, can impart high heat resistance, and is useful for improvements in resolution performance and roughness of a resist pattern (see, for example, NPL 3).

Lithography by electron beam or extreme ultraviolet light (EUV) is different in reaction mechanism from usual photolithography. Furthermore, lithography by electron beam or EUV is targeted for formation of a fine pattern of several tens nanometers. As the dimension of a resist pattern is thus smaller, a resist material high in sensitivity to an exposure light source is demanded. In particular, a resist composition in lithography by EUV is required to be increased in sensitivity, in terms of throughput.

There has been proposed, as a resist material for improvements of these, for example, an inorganic resist material having titanium, hafnium, or zirconium (see, for example, PTL 4 and PTL 5).

### CITATION LIST

### Patent Literature

PTL 1: Japanese Patent Laid-Open No. 2005-326838
PTL 2: Japanese Patent Laid-Open No. 2008-145539
PTL 3: Japanese Patent Laid-Open No. 2009-173623
PTL 4: Japanese Patent Laid-Open No. 2015-75500
PTL 5: Japanese Patent Laid-Open No. 2015-108781

### Non Patent Literature

NPL 1: Shinji Okazaki and other eight authors, “Development of Lithography Technology in These 40 Years”, S&T Publishing Inc., Dec. 9, 2016
NPL 2: T. Nakayama, M. Nomura. K. Haga, M. Ueda: Bull. Chem. Soc. Jpn., 71, 2979 (1998)
NPL 3: Shinji Okazaki and other twenty two authors, “New Trends of Photoresist Materials”, CMC Publishing Co., Ltd., September 2009, pp. 211-259

### SUMMARY OF INVENTION

### Technical Problem

However, inorganic resist materials are low in sensitivity and short in usable time. There is a need for a further increase in resolution, also in terms of resolution.

An object of the present invention is to provide a compound which is high in sensitivity and which provides high resolution and high flatness, and a method for producing the compound, an acid generating agent, a composition including the compound or the acid generating agent, a resist film, an underlayer film, an optical article, as well as a method for forming a pattern by use of the compound or the acid generating agent.

### Solution to Problem

The present inventors have made intensive studies in order to solve the above problems, and as a result, have found that the above problems can be solved by a specified compound or acid generating agent, leading to completion of the present invention. In other words, the present invention is as follows.

[1] A compound represented by the following formula (P-0).

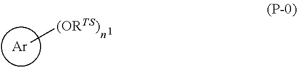

In formula (P-0), Ar is a group having an aryl group having 6 to 60 carbon atoms, each OR^{TS} is independently a hydroxy group, a group represented by the following formula (TS-0), or a group represented by the following formula (TS-1), and n¹ is an integer of 1 to 20, provided that at least one OR^{TS} is a group represented by the following formula (TS-0) or a group represented by the following formula (TS-1).

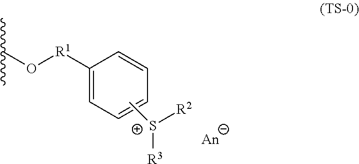

In formula (TS-0), R¹ is a single bond or a divalent group having 1 to 30 carbon atoms and optionally having a substituent. R² is an alkyl group having 1 to 10 carbon atoms and optionally having a substituent or an aryl group having 6 to 10 carbon atoms and optionally having a substituent, R³ is an alkyl group having 1 to 10 carbon atoms and optionally having a substituent or an aryl group having 6 to 10 carbon atoms and optionally having a substituent, and An⁻ is an anion containing fluorine or iodine.

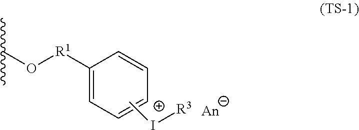

In formula (TS-1), R¹, R³ and An⁻ have the same meanings as in formula (TS-0).

[2] The compound according to [1], wherein, in formula (TS-0) and formula (TS-1). R³ is an alkyl group having 1 to 10 carbon atoms and optionally having a substituent, and An⁻ is R⁴SO₃⁻, wherein R⁴ is a monovalent group containing fluorine or iodine, having 1 to 9 carbon atoms and optionally having a substituent.

[3] The compound according to [1] or [2], wherein R¹ in formula (TS-0) and formula (TS-1) is a divalent group having 2 to 6 carbon atoms and optionally having a substituent.

[4] The compound according to any one of [1] to [3], wherein R² in formula (TS-0) is a methyl group or an ethyl group.

[5] The compound according to [4], wherein R² in formula (TS-0) is a methyl group.

[6] The compound according to any of [1] to [5], wherein, in formula (TS-0)) and formula (TS-1). R³ is a methyl group and An⁻ is CF₃SO₃⁻.

[7] The compound according to any of [1] to [6], wherein the compound is a compound represented by the following formula (P-0A).

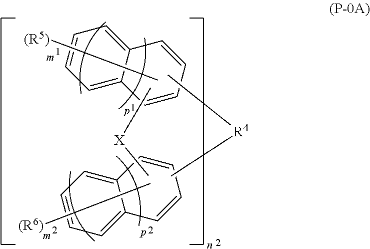

In formula (P-0A), each X is independently an oxygen atom, a sulfur atom or non-crosslinking. R⁴ is a single bond or a 2n-valent group having 1 to 30 carbon atoms and optionally having a substituent, R⁵ and R⁶ are each independently a halogen atom, a straight alkyl group having 1 to 30 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a cyano group, a nitro group, an amino group, a carboxylic acid group, a thiol group, a hydroxy group, the group represented by formula (TS-0), or the group represented by formula (TS-1), wherein the alkyl group, the aryl group, the alkenyl group, the alkynyl group, and the alkoxy group each optionally contain an ether bond, a ketone bond or an ester bond, m¹ and m² are each independently an integer of 0 to 7, p¹ and p² are each independently 0 or 1, and n² is an integer of 1 to 4, provided that at least one of m¹ and m² is an integer of 1 to 7 and formula (P-0A) contains at least one of the group represented by formula (TS-0) or the group represented by formula (TS-1) as R or R⁶.

[8] The compound according to any of [1] to [6], wherein the compound is a compound represented by the following formula (P-0B).

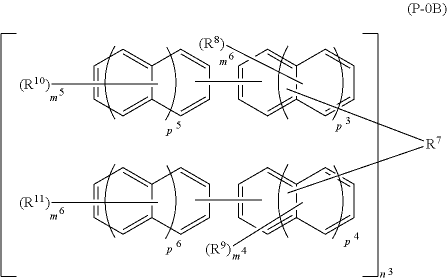

In formula (P-0B), R is a 2n-valent group having 1 to 30 carbon atoms, R⁸ to R¹¹ are each independently a halogen atom, a straight alkyl group having 1 to 10 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a thiol group, a cyano group, a nitro group, an amino group, a carboxylic acid group, a hydroxy group, the group represented by formula (TS-0), or the group represented by formula (TS-1), wherein the alkyl group, the aryl group, the alkenyl group, the alkynyl group, and the alkoxy group each optionally contain an ether bond, a ketone bond or an ester bond, m³ and m⁴ are each independently an integer of 0 to 8, m⁵ and m⁶ are each independently an integer of 0 to 9, p³ to p⁶ are each independently an integer of 0 to 2, and n³ is an integer of 1 to 4, provided that at least one of m³, m⁴, m⁵ and m⁶ is an integer of 1 or more and formula (P-0B) contains at least one of the group represented by formula (TS-0) or the group represented by formula (TS-1), as R⁸, R⁹, R¹⁰ or R¹¹.

[9] The compound according to any of [1] to [6], wherein the compound is a compound represented by the following formula (P-0C).

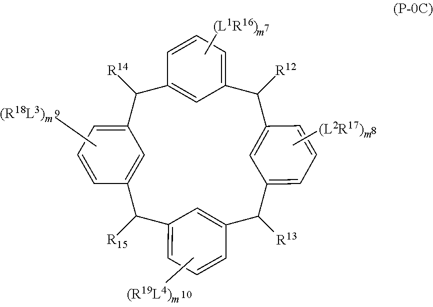

In formula (P-0C), L¹ to L⁴ are each independently a single bond, a straight alkylene group having 1 to 20 carbon atoms and optionally having a substituent, a branched alkylene group having 3 to 20 carbon atoms and optionally having a substituent, a cycloalkylene group having 3 to 20 carbon atoms and optionally having a substituent, an arylene group having 6 to 24 carbon atoms and optionally having a substituent, —O—, —OC(═O)—, —OC(O)O—, —N(R²⁰)—C(═O)—, —N(R²⁰)—C(═O)O—, —S—, —SO—, or —SO₂—, R²⁰ is a hydrogen atom or an alky group having 1 to 10 carbon atoms and optionally having a substituent, R¹⁶ to R¹⁹ are each independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, the group represented by formula (TS-0), the group represented by formula (TS-1), a cyano group, a nitro group, a hydroxy group, a heterocyclic group, a halogen atom, a carboxyl group, an alkylsilyl group having 1 to 20 carbon atoms; a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid; or a hydrogen atom, R¹² to R¹⁵ are each independently an alkyl group having 2 to 20 carbon atoms, the group represented by formula (TS-0), the group represented by formula (TS-1), or a group represented by the following formula (P-0C-1).

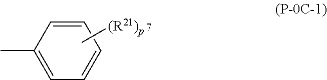

Each R²¹ is independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, a cyano group, a nitro group, a heterocyclic group, a halogen atom, a carboxyl group, an alkylsilyl group having 1 to 20 carbon atoms, or a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid, provided that at least one of R¹² to R¹⁹ is the group represented by formula (TS-0) or the group represented by formula (TS-1); m⁷ to m¹⁰ are each independently an integer of 1 to 4, and p⁷ is an integer of 0 to 5.

[10] The compound according to any of [1] to [6], wherein the compound is a compound represented by the following formula (P-1).

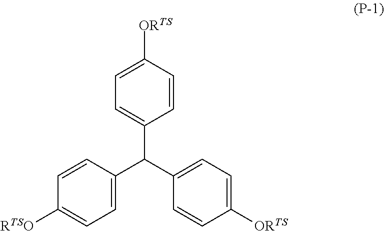

In formula (P-1), OR^{TS} has the same meaning as in formula (P-0).

[11] A composition comprising the compound according to any of [1] to [10].

[12] The composition according to [11], further comprising a solvent.

[13] The composition according to [11] or [12], further comprising an acid generating agent.

[14] The composition according to any of [11] to [13], further comprising an acid crosslinking agent.

[15] A resist film formed from the composition according to any of [11] to [14].

[16] A method for forming a pattern, comprising

a film formation step of forming a film on a substrate by use of the composition according to any of [11] to [14],

an exposure step of exposing the film, and

a development step of developing the film exposed in the exposure step, to thereby form a pattern.

[17] A method for producing the compound according to any of [1] to [10], comprising

a step of condensing a compound represented by the following formula (P-0′), and a compound represented by the following formula (TS-0′) or a compound represented by the following formula (TS-1′) to thereby obtain a condensate, and

a step of reacting the condensate, a salt having an anion containing fluorine or iodine, and an alkylating agent.

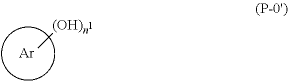

In formula (P-0′), Ar and n¹ have the same meanings as in formula (P-0).

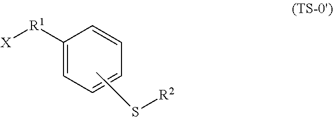

In formula (TS-0′), X is a halogen atom, and R¹ and R² have the same meanings as in formula (TS-0).

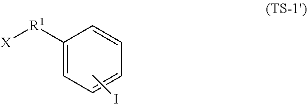

In formula (TS-1′), X is a halogen atom and R¹ has the same meaning as in formula (TS-1).

[18] An acid generating agent comprising the compound according to any of [1] to [10].

[19] A composition comprising the acid generating agent according to [18].

[20] The composition according to [19], further comprising a solvent.

[21] The composition according to [19] or [20], further comprising an acid crosslinking agent.

[22] The composition according to any of [19] to [21], wherein the composition is a composition for underlayer film formation for lithography.

[23] The composition according to [22], further comprising a silicon-containing compound.

[24] An underlayer film formed from the composition according to [22] or [23].

[25] A method for forming a pattern, comprising

a step of forming a resist underlayer film by use of the composition according to [22] or [23],

a step of forming at least one photoresist layer on the resist underlayer film, and

a step of irradiating a predetermined region of the photoresist layer with radiation to thereby perform development.

[26] The composition according to any of [19] to [21], wherein the composition is a composition for optical article formation.

[27] An optical article formed from the composition according to [26].

### Advantageous Effects of Invention

According to the present invention, there can be provided a compound which is high in sensitivity and which provides high resolution and high flatness, and a method for producing the compound, an acid generating agent, a composition including the compound or the acid generating agent, a resist film, an underlayer film, an optical article, as well as a method for forming a pattern by use of the compound or the acid generating agent.

### BRIEF DESCRIPTION OF DRAWINGS

FIG. 1 A ¹H-NMR spectrum of BEPMS in Example 1.

FIG. 2 A ¹H-NMR spectrum of MTP-BEPMS in Example 1.

FIG. 3 A ¹H-NMR spectrum of an MTP-BEPMS ion compound in Example 1.

FIG. 4 A ¹H-NMR spectrum of BHPMS in Example 8.

FIG. 5 A ¹H-NMR spectrum of MTP-BHPMS in Example 8.

FIG. 6 A ¹H-NMR spectrum of an MTP-BHPMS ion compound in Example 8.

### DESCRIPTION OF EMBODIMENTS

Hereinafter, embodiments of the present invention will be described (hereinafter, sometimes referred to as “the present embodiment”). Herein, the present embodiment is illustrative for describing the present invention, and the present invention is not limited to only the present embodiment.

[Compound]

A compound according to the present embodiment is represented by the following formula (P-0).

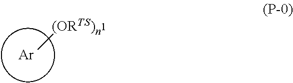

In formula (P-0), Ar is a group having an aryl group having 6 to 60 carbon atoms, each OR^{TS} is independently a hydroxy group, a group represented by the following formula (TS-0), or a group represented by the following formula (TS-1), and n¹ is an integer of 1 to 20, provided that at least one OR's is a group represented by the following formula (TS-0) or a group represented by the following formula (TS-1).

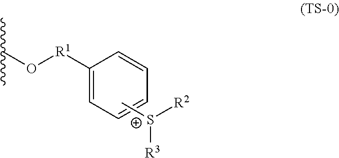

In formula (TS-0), R¹ is a single bond or a divalent group having 1 to 30 carbon atoms and optionally having a substituent, R² is an alkyl group having 1 to 10 carbon atoms and optionally having a substituent or an aryl group having 6 to 10 carbon atoms and optionally having a substituent, R³ is an alkyl group having 1 to 10 carbon atoms and optionally having a substituent or an aryl group having 6 to 10 carbon atoms and optionally having a substituent, and An⁻ is an anion containing fluorine or iodine.

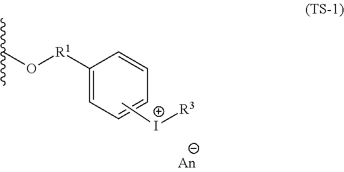

In formula (TS-1), R¹, R³ and An⁻ have the same meanings as in formula (TS-0).

A chemical structure of the compound according to the present embodiment can be confirmed by ¹H-NMR measurement and IR measurement. The compound has an ion moiety having a specified structure at a terminal, and thus, when used as a resist material or the like, exhibits high sensitivity and provides high resolution and high flatness. The rate of diffusion of a molecule of the compound according to the present embodiment is appropriate, and thus high resolution is exhibited with high sensitivity being kept. The compound has an appropriate molecular weight, thus is hardly volatilized and allows for relatively low film reduction in curing, and thus allows high flatness to be exhibited.

Herein, unless otherwise defined, the “substituted” means that one or more hydrogen atoms in a functional group is/are each substituted with a substituent. Examples of the “substituent” include, but not particularly limited, a halogen atom, a hydroxy group, a cyano group, a nitro group, an amino group, a thiol group, a heterocyclic group, a straight aliphatic hydrocarbon group having 1 to 20 carbon atoms, a branched aliphatic hydrocarbon group having 3 to 20 carbon atoms, a cyclic aliphatic hydrocarbon group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 20 carbon atoms, an alkoxycarbonyl group having 2 to 20 carbon atoms, an alkyloyloxy group having 1 to 20 carbon atoms, an aryloyloxy group having 7 to 30 carbon atoms or an alkylsilyl group having 1 to 20 carbon atoms.

In formula (P-0), Ar is a group having an aryl group having 6 to 60 carbon atoms. Ar preferably has 5 to 40 carbon atoms. Examples of Ar include phenyl, naphthyl, anthrathyl, biphenyl, fluorene, and any group containing such Ar. In formula (P-0), each OR^{TS} is independently a hydroxy group, the group represented by formula (TS-0), or the group represented by formula (TS-1). n¹ is an integer of 1 to 20, and at least one OR^{TS} is the group represented by formula (TS-0) or the group represented by formula (TS-1). In other words, formula (P-0) contains at least one of the group represented by formula (TS-0) or the group represented by formula (TS-1). n¹ is preferably 1 to 4.

In formula (TS-0), R¹ is a single bond, or a divalent group having 1 to 30 carbon atoms and optionally having a substituent, and is preferably a divalent group having 2 to 6 carbon atoms and optionally having a substituent. Examples of the divalent group having 2 to 6 carbon atoms include an alkyleneoxy group having 2 to 6 carbon atoms, such as an ethyleneoxy group and a propyleneoxy group, and a phenylene group. In formula (TS-0), R² is an alkyl group having 1 to 10 carbon atoms and optionally having a substituent or an aryl group having 6 to 10 carbon atoms and optionally having a substituent. Examples of the alkyl group having 1 to 10 carbon atoms include a methyl group, an ethyl group, a propyl group, an isopropyl group, a butyl group, a t-butyl group, a decyl group, and a cyclohexyl group. Examples of the aryl group having 6 to 10 carbon atoms include a phenyl group and a naphthyl group. In particular. R² is preferably a methyl group, an ethyl group, or a phenyl group, more preferably a methyl group.

In formula (TS-0), R³ is an alkyl group having 1 to 10 carbon atoms and optionally having a substituent or an aryl group having 6 to 10 carbon atoms and optionally having a substituent, and is preferably an alkyl group having 1 to 10 carbon atoms and optionally having a substituent. Examples of the alkyl group having 1 to 10 carbon atoms include a methyl group, an ethyl group, a propyl group, an isopropyl group, a butyl group, a t-butyl group, a decyl group, and a cyclohexyl group, and a methyl group is preferable. In formula (TS-0), An⁻ is an anion containing fluorine or iodine, and is preferably R⁴SO₃⁻ (R⁴ is a monovalent group containing fluorine or iodine and having 1 to 9 carbon atoms), PF₆⁻ or SbF₆⁻. Examples of R⁴ include a trifluoromethyl group and a nonafluorobutyl group. In particular, An⁻ is preferably CF₃SO₃⁻.

In formula (TS-1), R¹, R³ and An⁻ have the same meanings as in formula (TS-0), and a group similar to that of formula (TS-0) is preferable.

The compound represented by formula (P-0) is preferably, for example, a compound represented by the following formula (P-0A).

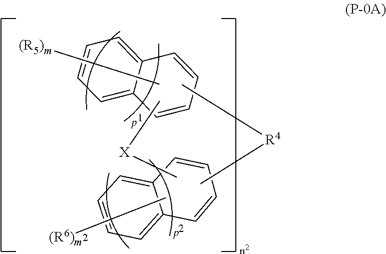

In formula (P-0A), each X is independently an oxygen atom, a sulfur atom or non-crosslinking, R⁴ is a single bond or a 2n-valent group having 1 to 30 carbon atoms and optionally having a substituent, R⁵ and R⁶ are each independently a halogen atom, a straight alkyl group having 1 to 30 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a cyano group, a nitro group, an amino group, a carboxylic acid group, a thiol group, a hydroxy group, the group represented by formula (TS-0), or the group represented by formula (TS-1), wherein the alkyl group, the aryl group, the alkenyl group, the alkynyl group, and the alkoxy group each optionally contain an ether bond, a ketone bond or an ester bond, m¹ and m² are each independently an integer of 0 to 7, p¹ and p² are each independently 0 or 1, and n² is an integer of 1 to 4, provided that at least one of m¹ and m² is an integer of 1 to 7 and formula (P-0A) contains at least one of the group represented by formula (TS-0) or the group represented by formula (TS-1) as R⁵ or R⁶.

In formula (P-0A), R⁴ is a single bond or a 2n-valent group having 1 to 30 carbon atoms and optionally having a substituent. The 2n-valent group having 1 to 30 carbon atoms is preferably a 2n-valent group having 1 to 16 carbon atoms, and examples thereof include a methylene group, a phenylmethylene group, a naphthylmethylene group, a biphenylmethylene group, a cyclohexylphenylmethylene group, an anthrathylmethylene group, and a biphenylethylene group. R⁴ is preferably a group represented by R^{A}-R^{B}, wherein R^{A} is a methine group and R^{B} is an aryl group having 5 to 29 carbon atoms and optionally having a substituent, and in this case, n² is 1.

In formula (P-0A), R⁵ and R⁶ are each independently a halogen atom, a straight alkyl group having 1 to 30 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a cyano group, a nitro group, an amino group, a carboxylic acid group, a thiol group, a hydroxy group, the group represented by formula (TS-0), or the group represented by formula (TS-1). The alkyl group, the aryl group, the alkenyl group, the alkynyl group, and the alkoxy group each optionally contain an ether bond, a ketone bond or an ester bond. Examples of the halogen atom include a fluorine atom, a chlorine atom, a bromine atom, and an iodine atom. Examples of the straight alkyl group having 1 to 30 carbon atoms include a methyl group, an ethyl group, a propyl group, a butyl group, and a decyl group. Examples of the branched alkyl group having 3 to 30 carbon atoms include an isopropyl group, an isobutyl group, and t-butyl. Examples of the cyclic alkyl group having 3 to 30 carbon atoms include a cyclopropyl group, a cyclobutyl group, a cyclopentyl group, a cyclohexyl group, a cyclodexyl group, and a nonahydronaphthyl group. Examples of the aryl group having 6 to 30 carbon atoms include a phenyl group and a naphthyl group. Examples of the alkenyl group having 2 to 30 carbon atoms include a vinyl group and an allyl group. Examples of the alkoxy group having 1 to 30 carbon atoms include a methoxy group, an ethoxy group, a propoxy group, a butoxy group, and a triacontyloxy group.

In formula (P-0A), m¹ and m² are each independently an integer of 0 to 7, preferably an integer of 1 to 7, provided that at least one of m¹ and m² is an integer of 1 to 7 and formula (P-0A) contains at least one of the group represented by formula (TS-0) or the group represented by formula (TS-1) as R⁵ or R⁶. p¹ and p² are each independently 0 or 1. n² is an integer of 1 to 4, preferably an integer of 1 to 2.

Examples of the compound represented by formula (P-0A) include any compound disclosed in International Publication No. WO2013/024778, in which a hydroxy group (—OH) in the compound is substituted with a group represented by —OR^{TS}. Specific examples include the following compounds. Herein, the compound represented by formula (P-0A) is not limited to these specific compounds.

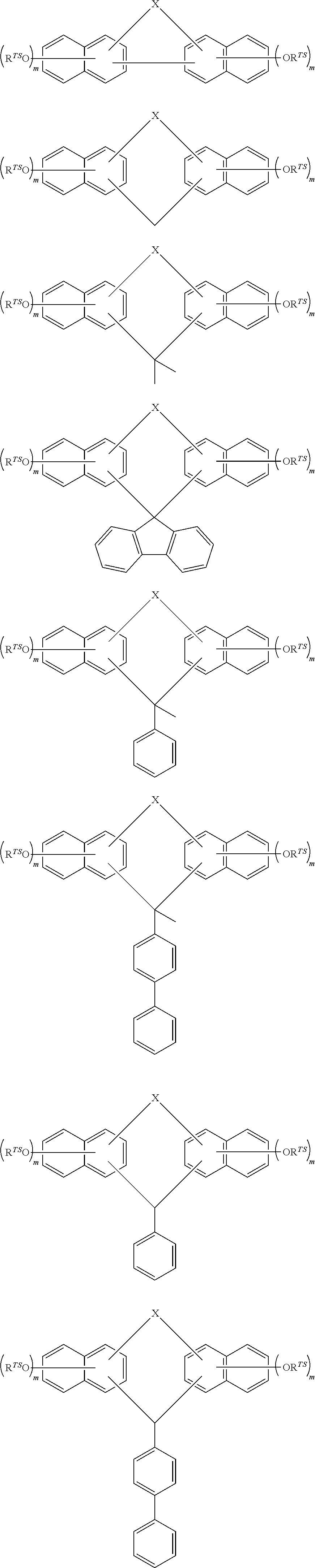

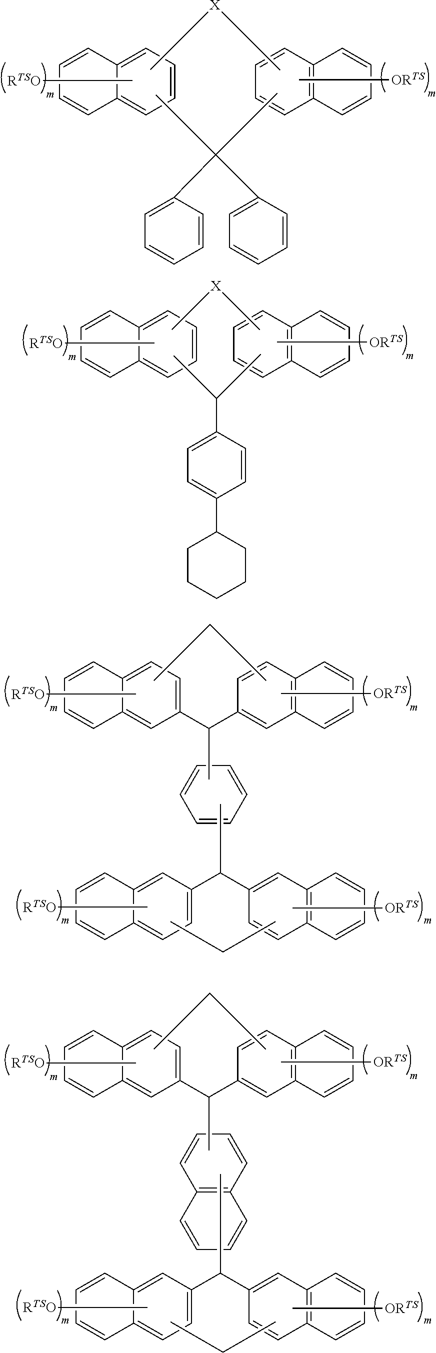

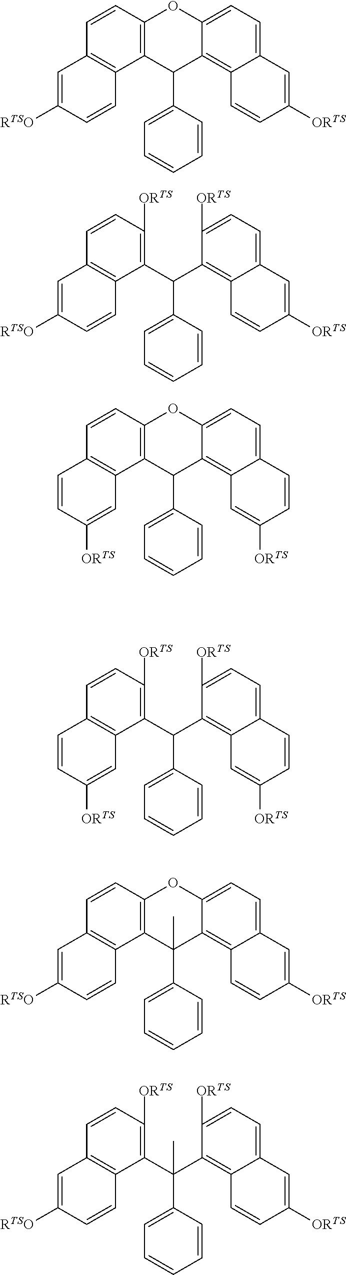

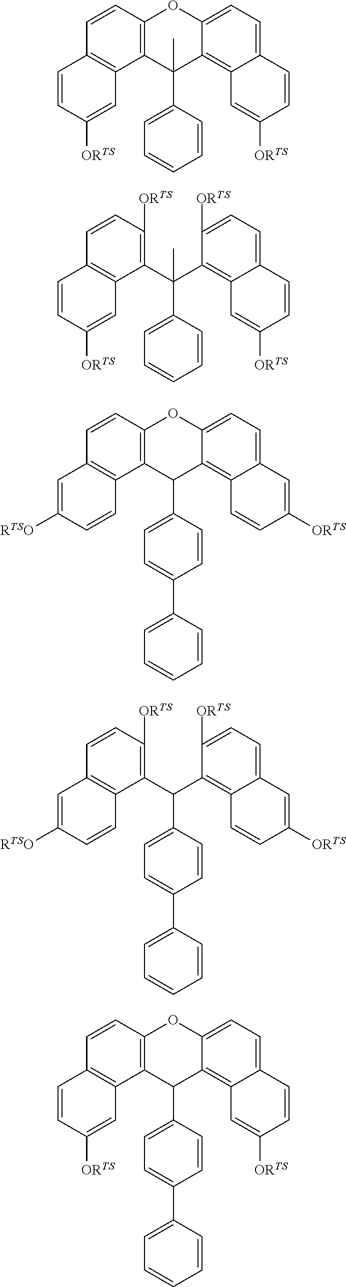

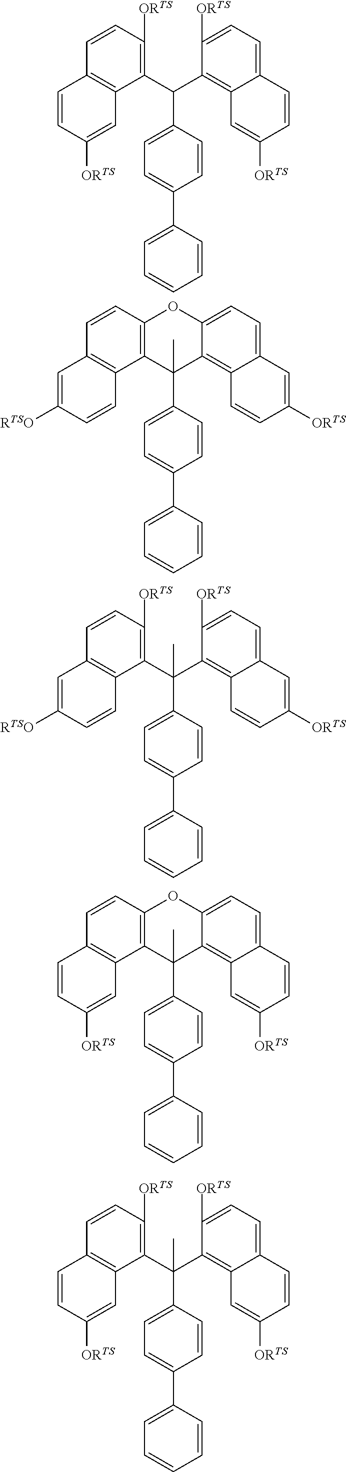

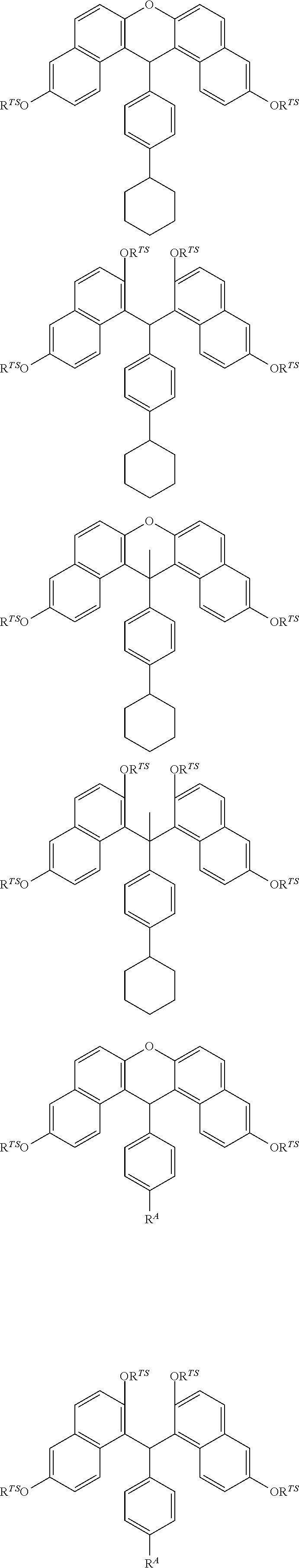

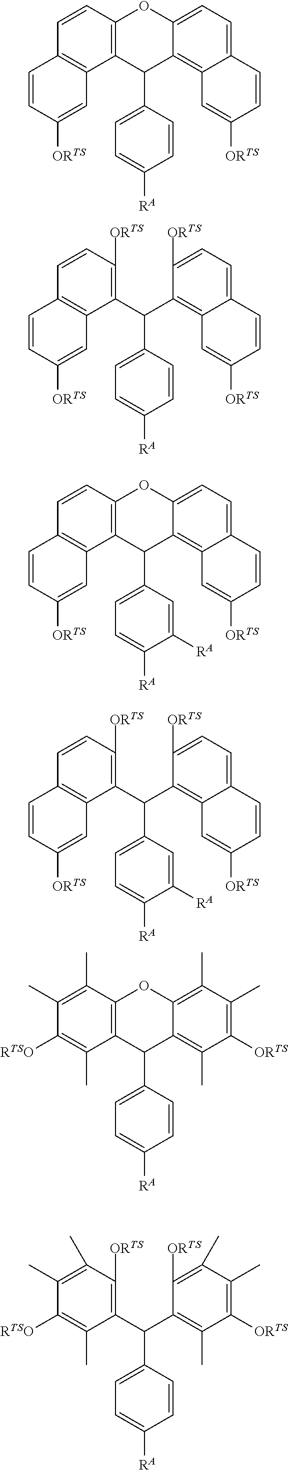

In the formulae, R^{A} is a fluorine atom, a chlorine atom, a bromine atom, an iodine atom, a hydroxy group, a cyano group, a nitro group, an amino group, a thiol group, a heterocyclic group, a straight aliphatic hydrocarbon group having 1 to 20 carbon atoms, a branched aliphatic hydrocarbon group having 3 to 20 carbon atoms, a cyclic aliphatic hydrocarbon group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 20 carbon atoms, an alkoxycarbonyl group having 2 to 20 carbon atoms, an alkyloyloxy group having 1 to 20 carbon atoms, an aryloyloxy group having 7 to 30 carbon atoms or an alkylsilyl group having 1 to 20 carbon atoms, and OR^{TS} has the same meaning as in formula (P-0). Examples of the straight aliphatic hydrocarbon group having 1 to 20 carbon atoms include a methyl group, an ethyl group, a propyl group, a butyl group, an octyl group, a nonyl group, a decyl group, and a dodecyl group.

The compound represented by formula (P-0) is preferably, for example, a compound represented by the following formula (P-0B).

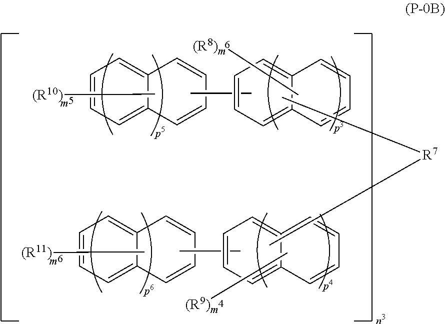

In formula (P-0B), R⁷ is a 2n-valent group having 1 to 30 carbon atoms, R⁸ to R¹¹ are each independently a halogen atom, a straight alkyl group having 1 to 10 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a thiol group, a cyano group, a nitro group, an amino group, a carboxylic acid group, a hydroxy group, the group represented by formula (TS-0), or the group represented by formula (TS-1), wherein the alkyl group, the aryl group, the alkenyl group, the alkynyl group, and the alkoxy group each optionally contain an ether bond, a ketone bond or an ester bond, m³ and m⁴ are each independently an integer of 0 to 8, m⁵ and m⁶ are each independently an integer of 0 to 9, p³ to p⁶ are each independently an integer of 0 to 2, and n¹ is an integer of 1 to 4, provided that at least one of m³, m⁴, m⁵ and m⁶ is an integer of 1 or more and formula (P-0B) contains at least one of the group represented by formula (TS-0) or the group represented by formula (TS-1), as R⁸, R⁹, R¹⁰ or R¹¹.

In formula (P-0B). R⁷ is a 2n-valent group having 1 to 30 carbon atoms, preferably a 2n-valent group having 1 to 16 carbon atoms, and examples thereof include a methylene group, a phenylmethylene group, a naphthylmethylene group, a biphenylmethylene group, a cyclohexylphenylmethylene group, an anthrathylmethylene group, and a biphenylethylene group. R⁷ is preferably a group represented by R^{A}—R^{B}, wherein R^{A} is a methine group and R^{B} is an aryl group having 5 to 29 carbon atoms and optionally having a substituent, and in this case, n² is 1.

In formula (P-0B), R⁸ to R¹¹ are each independently a halogen atom, a straight alkyl group having 1 to 10 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a thiol group, a cyano group, a nitro group, an amino group, a carboxylic acid group, a hydroxy group, the group represented by formula (TS-0), or the group represented by formula (TS-1). The alkyl group, the aryl group, the alkenyl group, the alkynyl group, and the alkoxy group each optionally contain an ether bond, a ketone bond or an ester bond. Examples of the halogen atom include a fluorine atom, a chlorine atom, a bromine atom, and an iodine atom. Examples of the straight alkyl group having 1 to 10 carbon atoms include a methyl group, an ethyl group, a propyl group, a butyl group, and a decyl group. Examples of the branched alkyl group having 3 to 30 carbon atoms include an isopropyl group, an isobutyl group, and t-butyl. Examples of the cyclic alkyl group having 3 to 30 carbon atoms include a cyclopropyl group, a cyclobutyl group, a cyclopentyl group, a cyclohexyl group, a cyclodexyl group, and a nonahydronaphthyl group. Examples of the aryl group having 6 to 30 carbon atoms include a phenyl group and a naphthyl group. Examples of the alkenyl group having 2 to 30 carbon atoms include a vinyl group and an allyl group. Examples of the alkoxy group having 1 to 30 carbon atoms include a methoxy group, an ethoxy group, a propoxy group, a butoxy group, and a triacontyloxy group.

In formula (P-0B), m³ and m⁴ are each independently an integer of 0 to 8, preferably an integer of 0 to 2, and m⁵ and m⁶ are each independently an integer of 0 to 9, preferably an integer of 0 to 2, provided that at least one of m³, m⁴, m⁵ and m⁶ is an integer of 1 or more and formula (P-0B) contains at least one of the group represented by formula (TS-0) or the group represented by formula (TS-1), as R⁸, R⁹, R¹⁰ or R¹¹. p³ to p⁶ are each independently an integer of 0 to 2, preferably an integer of 0 to 1. n³ is an integer of 1 to 4, preferably an integer of 1 to 2.

Examples of the compound represented by formula (P-0B) include any compound disclosed in International Publication No. WO2015/137486, in which a hydroxy group (—OH) in the compound is substituted with a group represented by —OR^{TS}, and specific examples include the following compounds. The compound represented by formula (P-0B) is not limited to these specific compounds.

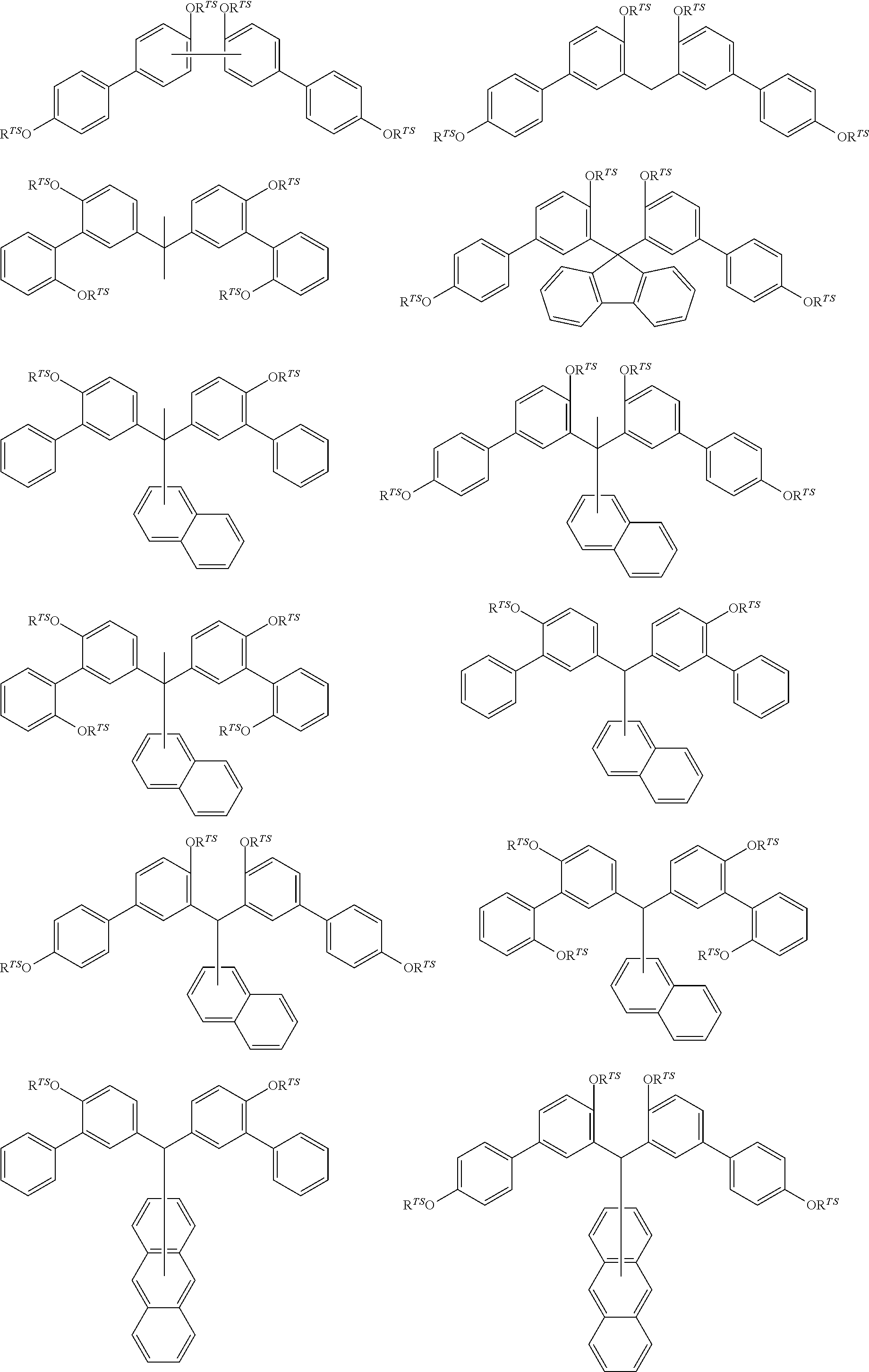

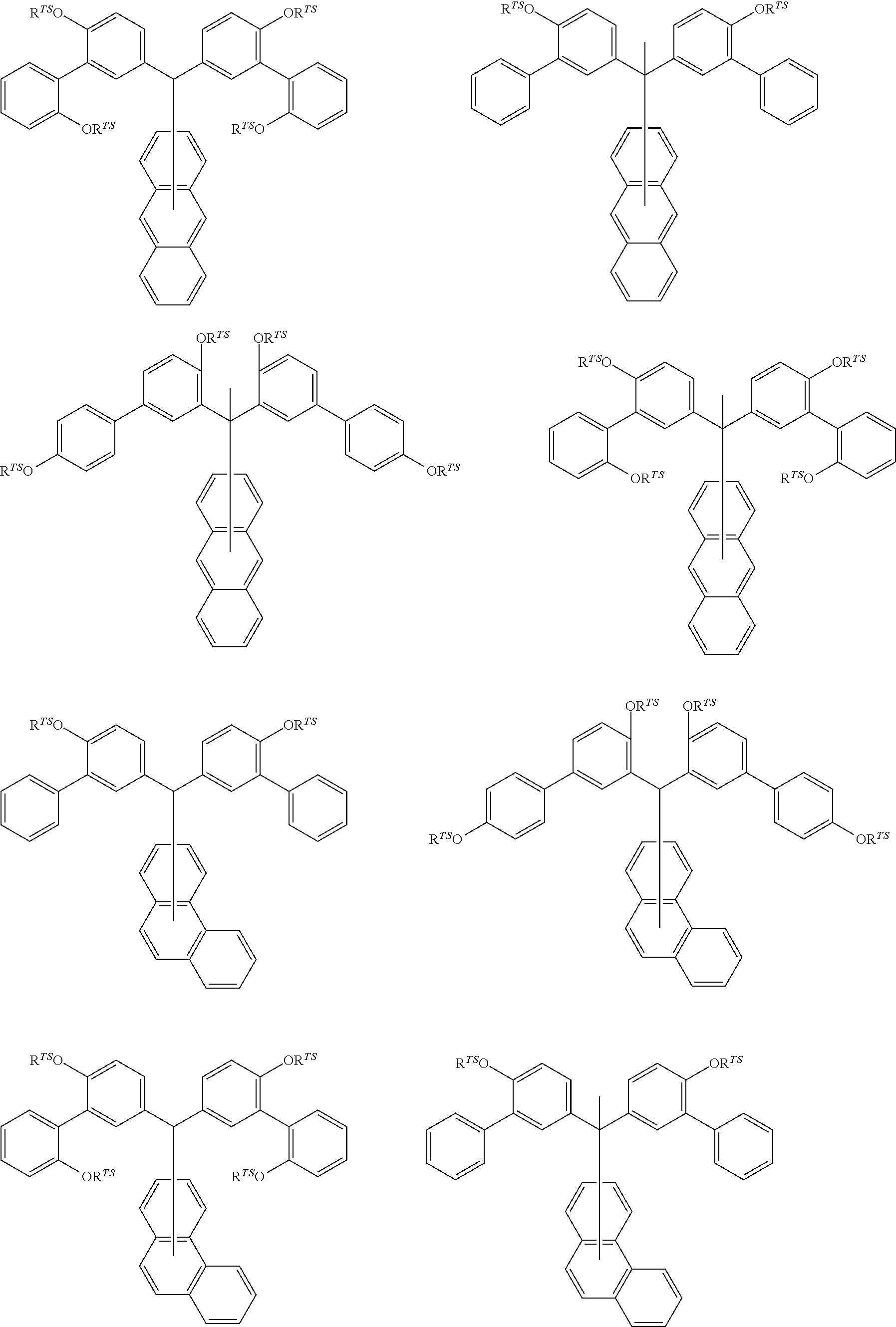

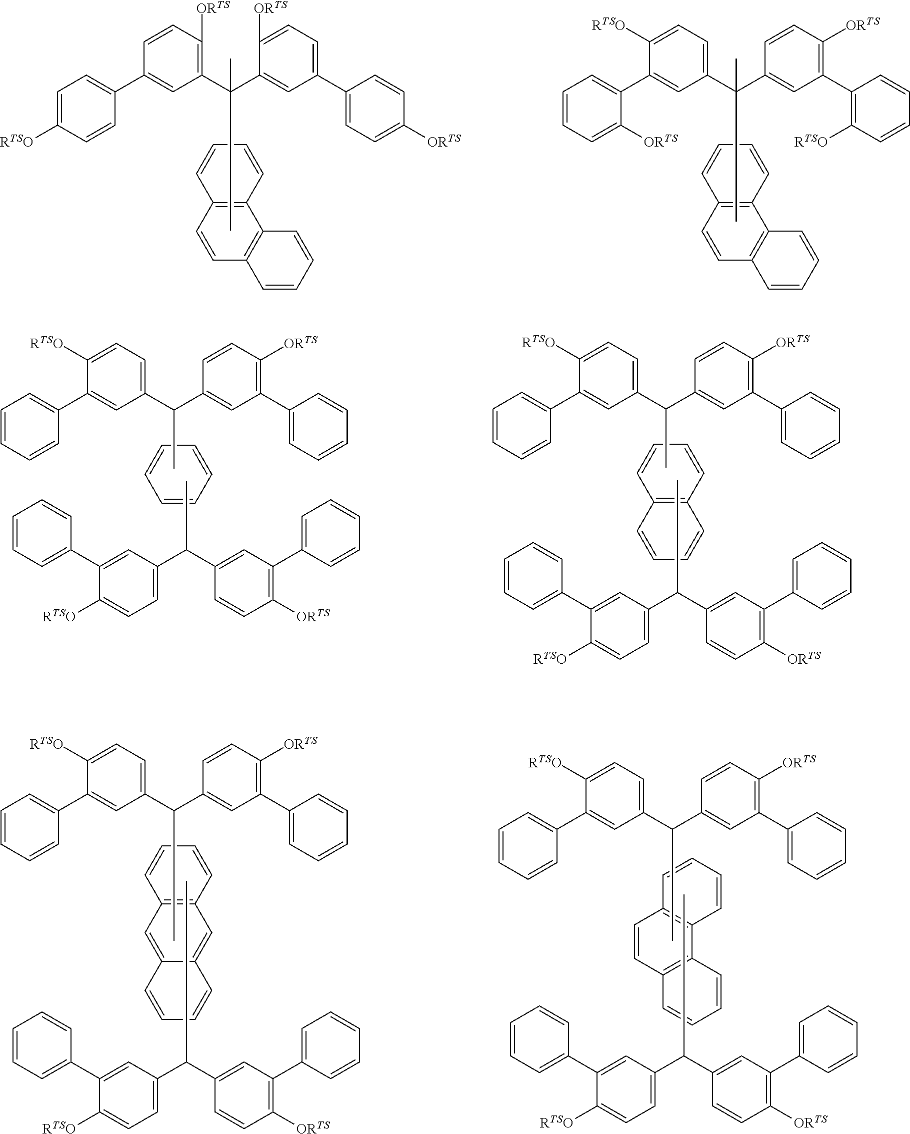

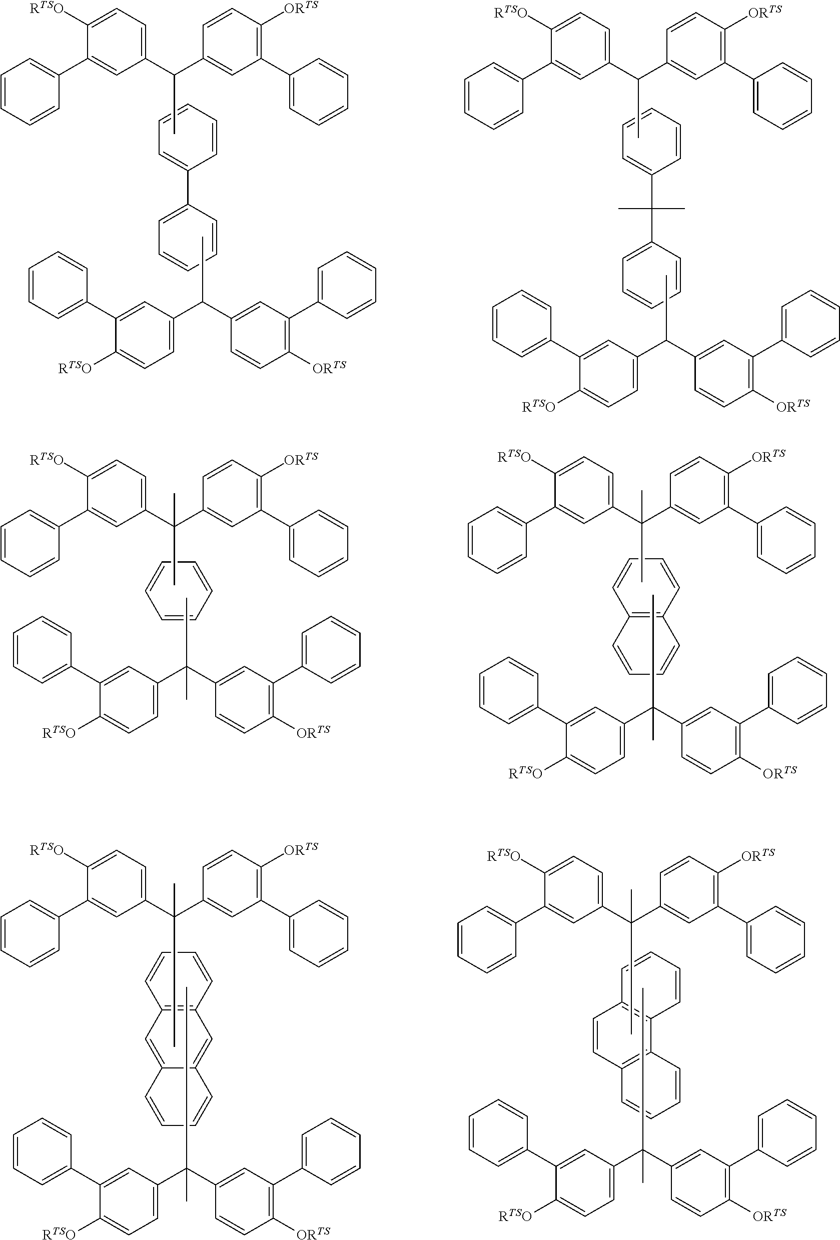

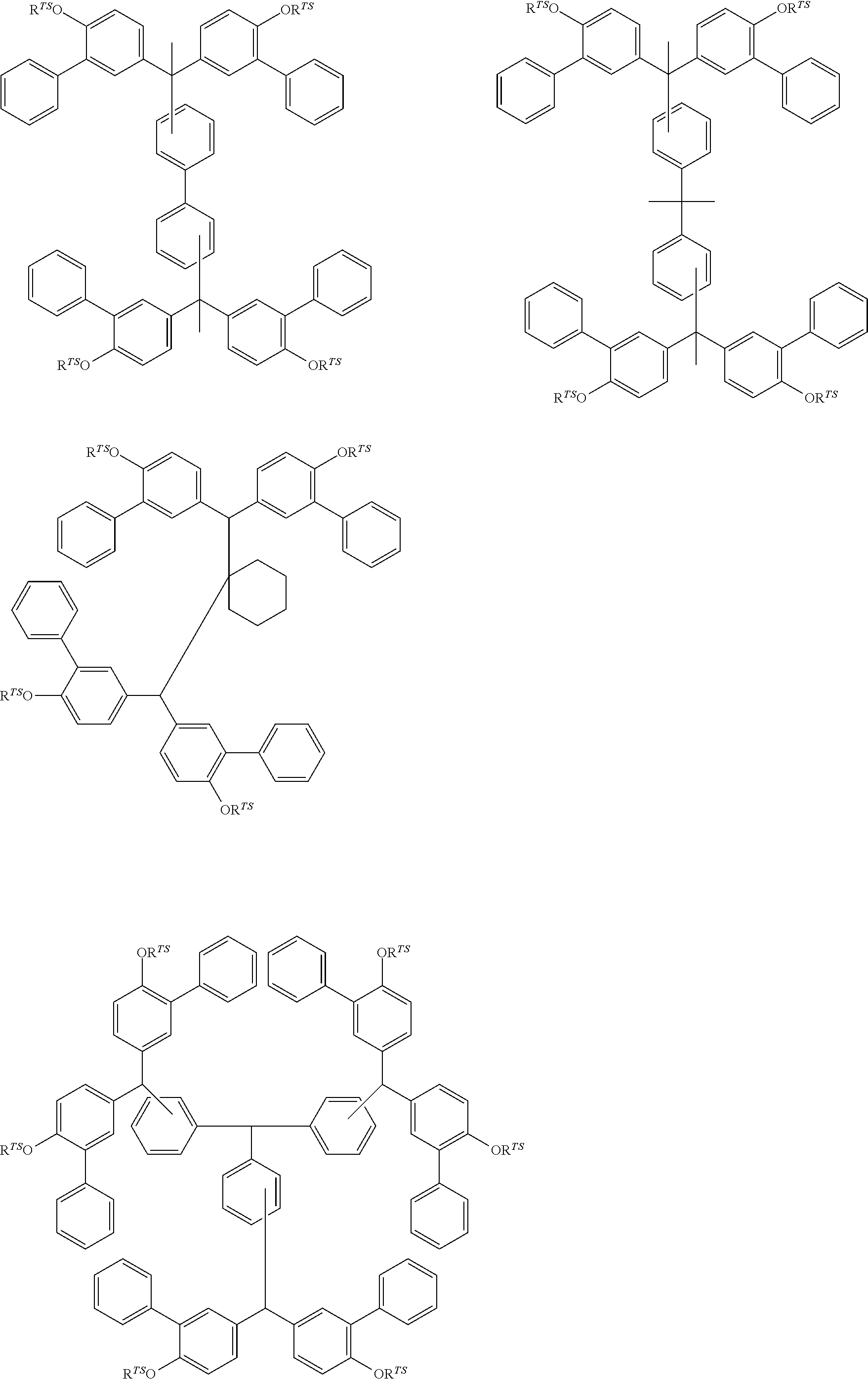

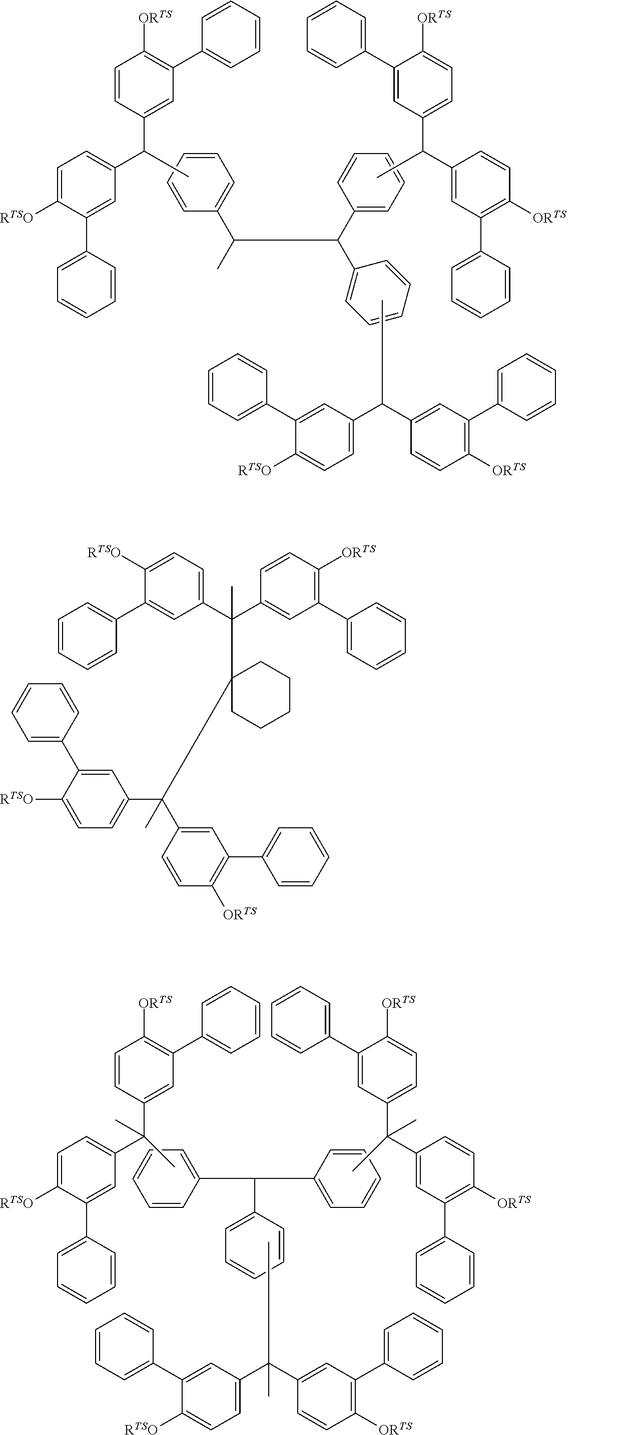

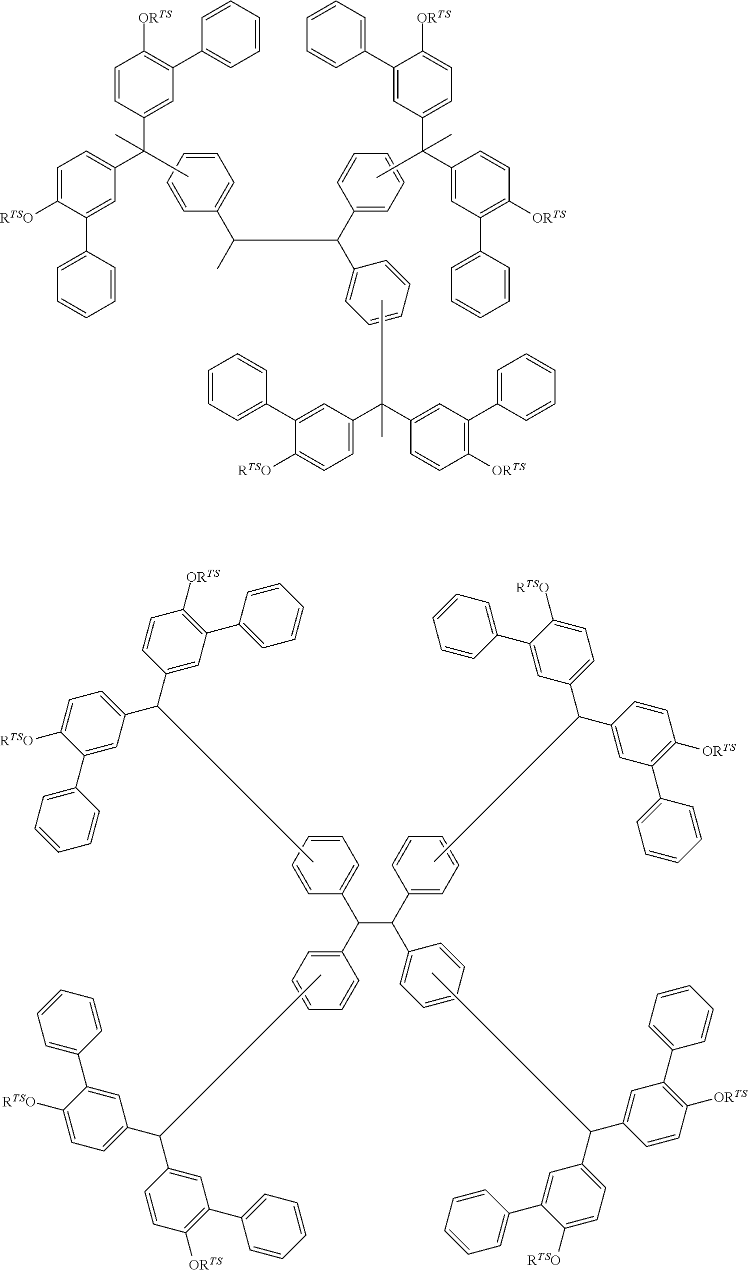

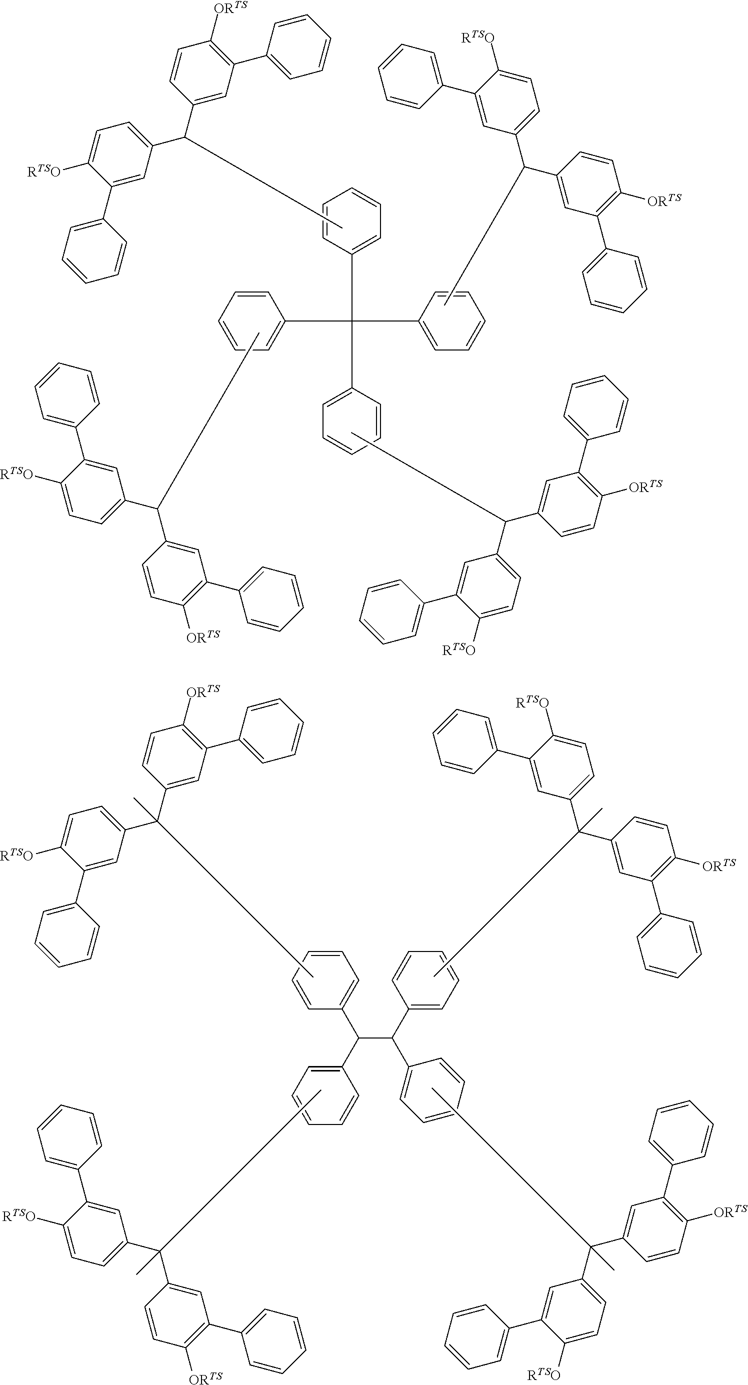

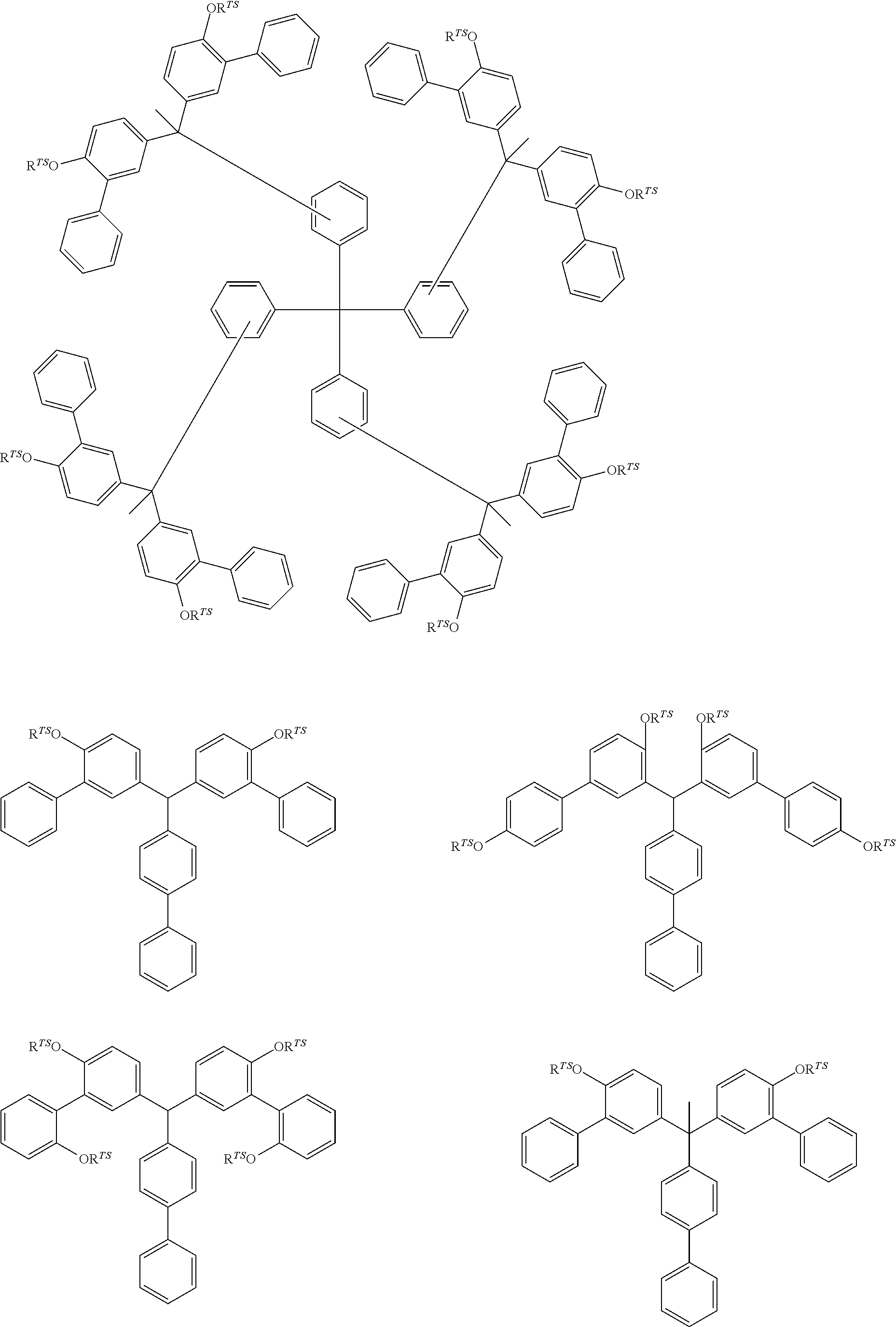

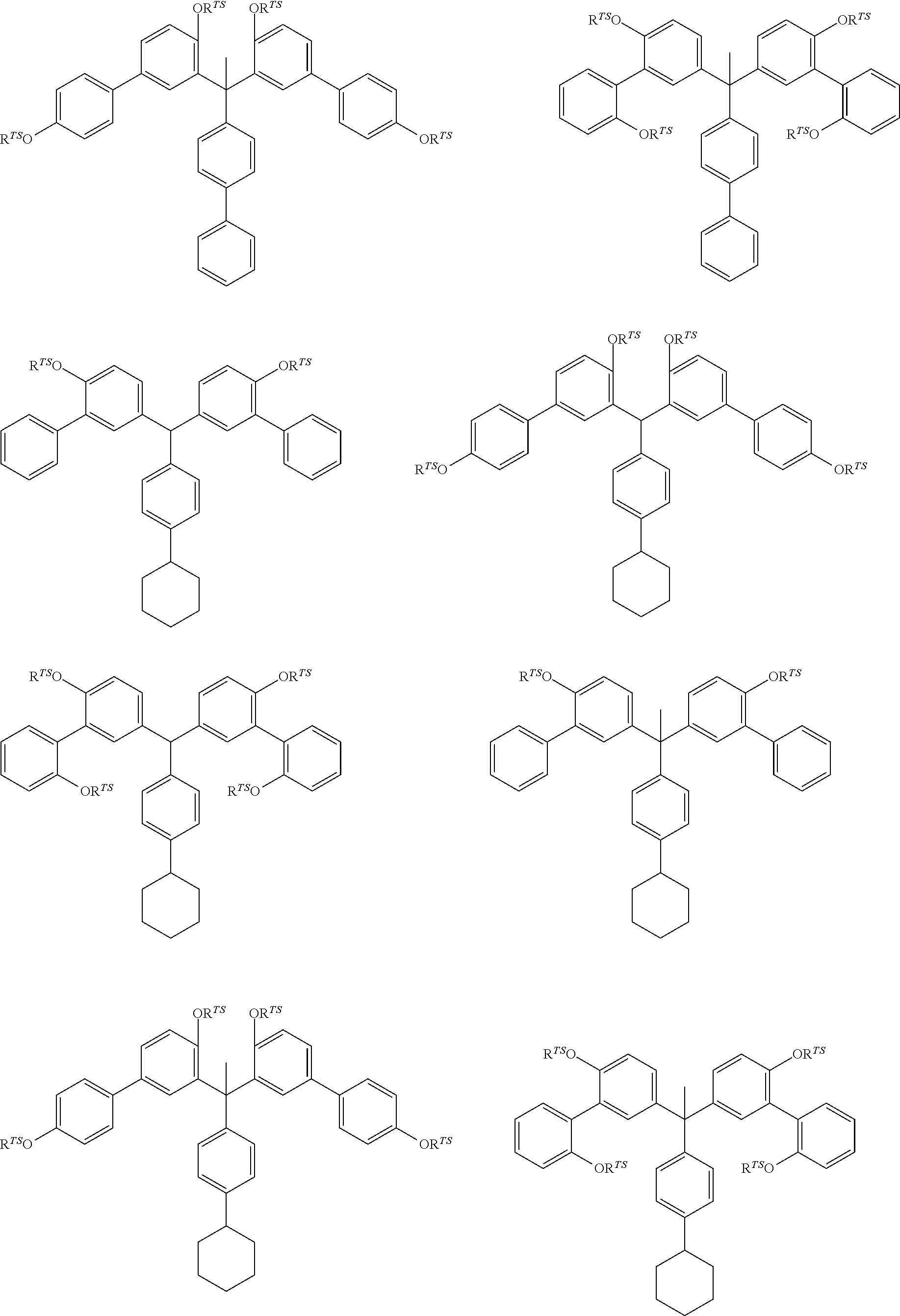

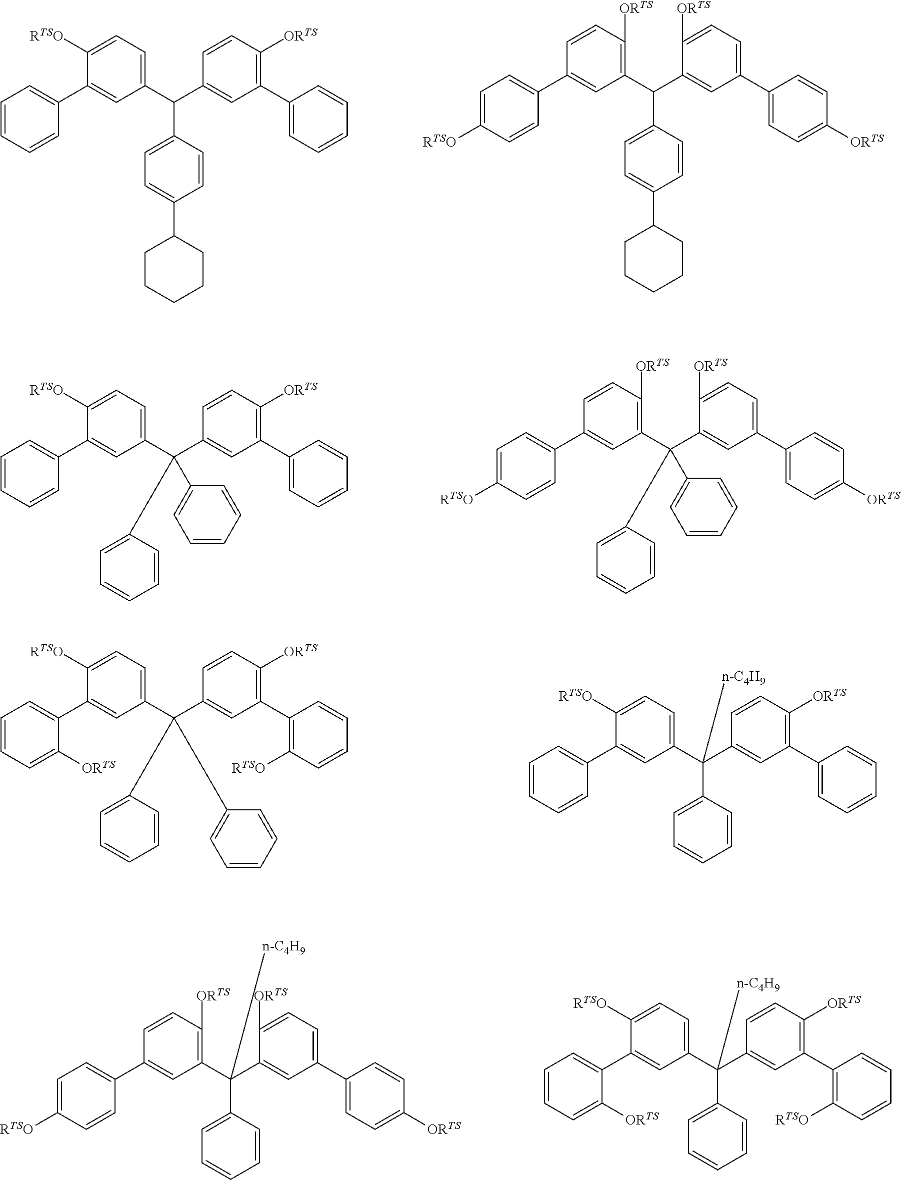

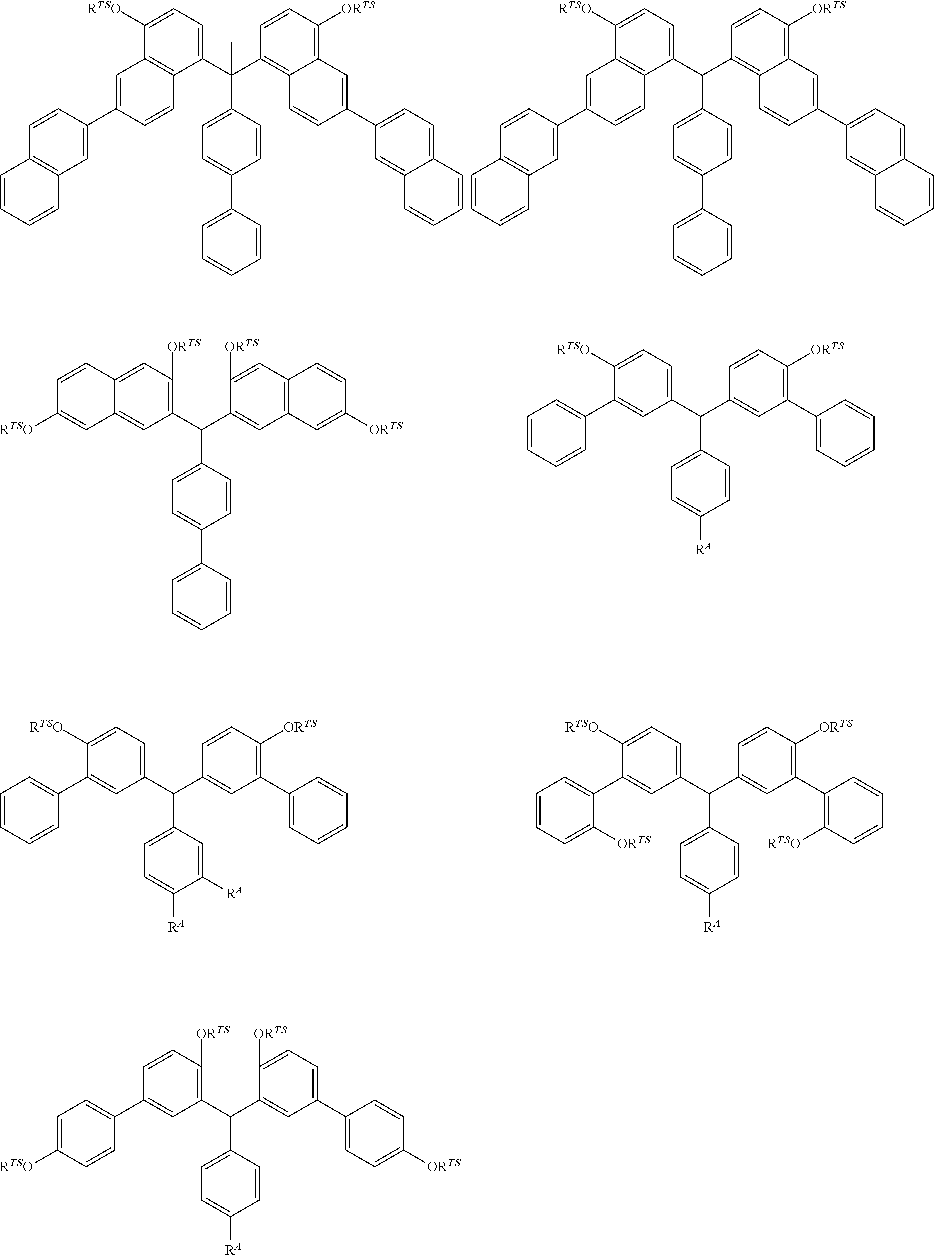

In the formulae, R^{A} is a fluorine atom, a chlorine atom, a bromine atom, an iodine atom, a hydroxy group, a cyano group, a nitro group, an amino group, a thiol group, a heterocyclic group, a straight aliphatic hydrocarbon group having 1 to 20 carbon atoms, a branched aliphatic hydrocarbon group having 3 to 20 carbon atoms, a cyclic aliphatic hydrocarbon group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 20 carbon atoms, an alkoxycarbonyl group having 2 to 20 carbon atoms, an alkyloyloxy group having 1 to 20 carbon atoms, an aryloyloxy group having 7 to 30 carbon atoms or an alkylsilyl group having 1 to 20 carbon atoms, and OR^{TS} has the same meaning as in formula (P-0). Examples of the straight aliphatic hydrocarbon group having 1 to 20 carbon atoms include a methyl group, an ethyl group, a propyl group, a butyl group, an octyl group, a nonyl group, a decyl group, and a dodecyl group.

The compound represented by formula (P-0) is preferably, for example, a compound represented by the following formula (P-0C).

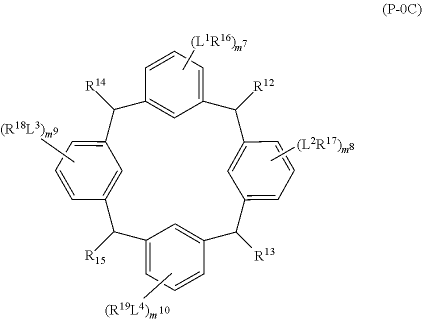

In formula (P-0C), L¹ to L⁴ are each independently a single bond, a straight alkylene group having 1 to 20 carbon atoms and optionally having a substituent, a branched alkylene group having 3 to 20 carbon atoms and optionally having a substituent, a cycloalkylene group having 3 to 20 carbon atoms and optionally having a substituent, an arylene group having 6 to 24 carbon atoms and optionally having a substituent, —O—, —OC(═O)—, —OC(O)O—, —N(R²⁰)—C(═O)—, —N(R²⁰)—C(═O)O—, —S—, —SO—, or —SO₂—, R²⁰ is a hydrogen atom or an alkyl group having 1 to 10 carbon atoms and optionally having a substituent, R¹ to R¹⁹ are each independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, the group represented by formula (TS-0), the group represented by formula (TS-1), a cyano group, a nitro group, a hydroxy group, a heterocyclic group, a halogen atom, a carboxyl group, an alkylsilyl group having 1 to 20 carbon atoms, a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid; or a hydrogen atom, R¹² to R¹⁵ are each independently an alkyl group having 2 to 20 carbon atoms, the group represented by formula (TS-0), the group represented by formula (TS-1), or a group represented by the following formula (P-0C-1):

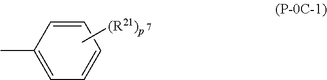

Each R²¹ is independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, a cyano group, a nitro group a heterocyclic group, a halogen atom, a carboxyl group, an alkylsilyl group having 1 to 20 carbon atoms, or a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid, provided that at least one of R¹² to R¹⁹ is the group represented by formula (TS-0) or the group represented by formula (TS-1); m⁷ to m¹⁰ are each independently an integer of 1 to 4, and p⁷ is an integer of 0 to 5.

In formula (P-0C), L¹ to L⁴ are each independently a single bond, a straight alkylene group having 1 to 20 carbon atoms and optionally having a substituent, a branched alkylene group having 3 to 20 carbon atoms and optionally having a substituent, a cycloalkylene group having 3 to 20 carbon atoms and optionally having a substituent, an arylene group having 6 to 24 carbon atoms and optionally having a substituent, —O—, —OC(═O)—, —OC(═O)O—, —N(R²⁰)—C(═O)—, —N(R²⁰)—C(═O)O—, —S—, —SO—, or —SO₂—. The straight alkylene group having 1 to 20 carbon atoms is preferably a straight alkylene group having 1 to 10 carbon atoms, and examples thereof include a methylene group, an ethylene group, a propylene group, and a decylene group. The branched alkylene group having 3 to 20 carbon atoms is preferably a branched alkylene group having 1 to 16 carbon atoms, and examples thereof include an isopropylene group, an isobutylene group, a phenylmethylene group, a naphthylmethylene group, a biphenylmethylene group, a cyclohexylphenylmethylene group, an anthrathylmethylene group, and a biphenylethylene group. The cycloalkylene group having 3 to 20 carbon atoms is preferably, for example, a cycloalkylene group having 3 to 10 carbon atoms, and examples thereof include a cyclopropylene group, a cyclobutylene group, a cyclopentylene group, a cyclohexylene group, a cyclodexylene group, and a nonahydronaphthylene group. The arylene group having 6 to 24 carbon atoms is preferably for example, an arylene group having 6 to 12 carbon atoms, and examples thereof include a phenylene group, a naphthylene group, and a biphenylene group. R²⁰ is a hydrogen atom, or an alkyl group having 1 to 10 carbon atoms and optionally having a substituent. Examples of the alkyl group having 1 to 10 carbon atoms include a methyl group, an ethyl group, a propyl group, an isopropyl group, a butyl group, an isobutyl group, and a t-butyl group.

In formula (P-0C), R¹⁶ to R¹⁹ are each independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, the group represented by formula (TS-0), the group represented by formula (TS-1), a cyano group, a nitro group, a hydroxy group, a heterocyclic group, a halogen atom, a carboxyl group, or an alkylsilyl group having 1 to 20 carbon atoms, a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid; or a hydrogen atom. The alkyl group having 1 to 20 carbon atoms is preferably an alkyl group having 1 to 10 carbon atoms, and examples thereof include a methyl group, an ethyl group, a propyl group, an isopropyl group, a butyl group, an isobutyl group, and a t-butyl group. The cycloalkyl group having 3 to 20 carbon atoms is preferably a cycloalkyl group having 1 to 10 carbon atoms, and examples thereof include a cyclopropyl group, a cyclobutyl group, a cyclopentyl group, a cyclohexyl group, a cyclodexylene group, and a nonahydronaphthylene group. The aryl group having 6 to 20 carbon atoms is preferably an aryl group having 6 to 10 carbon atoms, and examples thereof include a phenyl group, a naphthyl group, and a biphenyl group. The alkoxy group having 1 to 20 carbon atoms is preferably an alkoxy group having 1 to 10 carbon atoms, and examples thereof include a methoxy group, an ethoxy group, a propoxy group, a butoxy group, and a dexyl group. Examples of the heterocyclic group include a pyrrole group, an imidazole group, and a carbazole group. Examples of the halogen atom include a fluorine atom, a chlorine atom, a bromine atom, and an iodine atom. The alkylsilyl group having 1 to 20 carbon atoms is preferably an alkylsilyl group having 1 to 9 carbon atoms, and examples thereof include a trimethylsilyl group, a triethylsilyl group, a triisopropylsilyl group, and a tert-butyldimethylsilyl group.

The substituted methyl group having 2 to 20 carbon atoms and having the property of being dissociated by an acid is preferably a substituted methyl group having 4 to 18 carbon atoms, more preferably a substituted methyl group having 6 to 16 carbon atoms. Specific examples of the substituted methyl group can include, but are not limited to, a methoxymethyl group, a methylthiomethyl group, an ethoxymethyl group, a n-propoxymethyl group, an isopropoxymethyl group, a n-butoxymethyl group, a t-butoxymethyl group, a 2-methylpropoxymethyl group, an ethylthiomethyl group, a methoxyethoxymethyl group, a phenyloxymethyl group, a 1-cyclopentyloxymethyl group, a 1-cyclohexyloxymethyl group, a benzylthiomethyl group, a phenacyl group, a 4-bromophenacyl group, a 4-methoxyphenacyl group, a piperonyl group, and a substituent group represented by the following formula (1). Specific examples of R^{2A} in the following formula (1) include, but are not limited to, a methyl group, an ethyl group, an isopropyl group, a n-propyl group, a t-butyl group, and a n-butyl group. R^{2A} in the following formula (1) is an alkyl group having 1 to 4 carbon atoms.

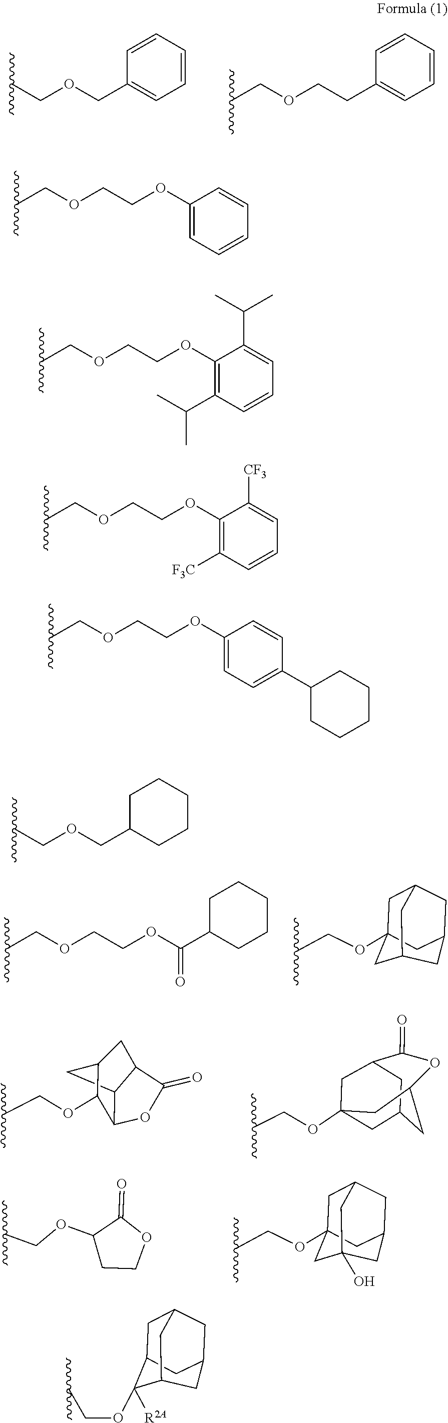

The 1-substituted ethyl group having 3 to 20 carbon atoms and having the property of being dissociated by an acid is preferably a 1-substituted ethyl group having 5 to 18 carbon atoms, more preferably a substituted ethyl group having 7 to 16 carbon atoms. Specific examples of the 1-substituted ethyl group can include, but are not limited to, a 1-methoxyethyl group, a 1-methylthioethyl group, a 1,1-dimethoxyethyl group, a 1-ethoxyethyl group, a 1-ethylthioethyl group, a 1,1-diethoxyethyl group, a n-propoxyethyl group, an isopropoxyethyl group, a n-butoxyethyl group, a t-butoxyethyl group, a 2-methylpropoxyethyl group, a 1-phenoxyethyl group, a 1-phenylthioethyl group, a 1,1-diphenoxyethyl group, a 1-cyclopentyloxyethyl group, a 1-cyclohexyloxyethyl group, a 1-phenylethyl group, a 1,1-diphenylethyl group, and a substituent group represented by the following formula (2). R^{2A} in the following formula (2) has the same meaning as in formula (1).

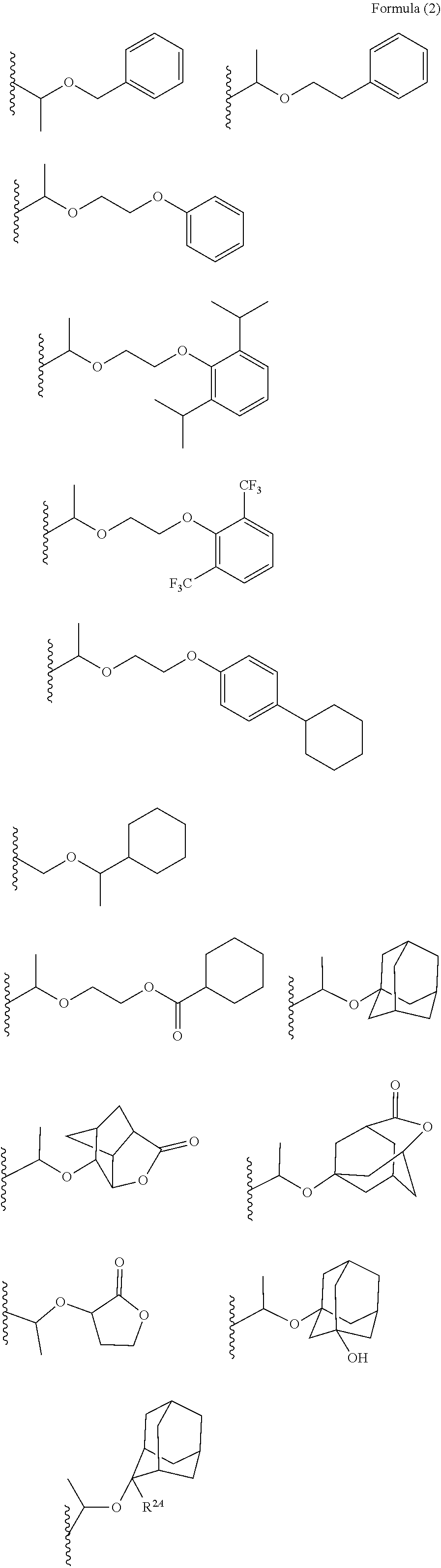

The 1-substituted-n-propyl group having 4 to 20 carbon atoms and having the property of being dissociated by an acid is preferably a 1-substituted-n-propyl group having 6 to 18 carbon atoms, more preferably a 1-substituted-n-propyl group having 8 to 16 carbon atoms. Specific examples of the 1-substituted-n-propyl group can include, but are not limited to, a 1-methoxy-n-propyl group and a 1-ethoxy-n-propyl group.

The 1-branched alkyl group having 3 to 20 carbon atoms and having the property of being dissociated by an acid is preferably a 1-branched alkyl group having 5 to 18 carbon atoms, more preferably a branched alkyl group having 7 to 16 carbon atoms. Specific examples of the 1-branched alkyl group can include, but are not limited to, an isopropyl group, a sec-butyl group, a tert-butyl group, a 1,1-dimethylpropyl group, a 1-methylbutyl group, a 1,1-dimethylbutyl group, a 2-methyladamantyl group, and a 2-ethyladamantyl group.

The silyl group having 1 to 20 carbon atoms and having the property of being dissociated by an acid is preferably a silyl group having 3 to 18 carbon atoms, more preferably a silyl group having 5 to 16 carbon atoms. Specific examples of the silyl group can include, but are not limited to, a trimethylsilyl group, an ethyldimethylsilyl group, a methyldiethylsilyl group, a triethylsilyl group, a tert-butyldimethylsilyl group, a tert-butyldiethylsilyl group, a tert-butyldiphenylsilyl group, a tri-tert-butylsilyl group and a triphenylsilyl group.

The acyl group having 2 to 20 carbon atoms and having the property of being dissociated by an acid is preferably an acyl group having 4 to 18 carbon atoms, more preferably an acyl group having 6 to 16 carbon atoms. Specific examples of the acyl group can include, but are not limited to, an acetyl group, a phenoxyacetyl group, a propionyl group, a butyryl group, a heptanoyl group, a hexanoyl group, a valeryl group, a pivaloyl group, an isovaleryl group, a lauryloyl group, an adamantylcarbonyl group, a benzoyl group, and a naphthoyl group.

The 1-substituted alkoxyalkyl group having 2 to 20 carbon atoms and having the property of being dissociated by an acid is preferably a 1-substituted alkoxymethyl group having 2 to 20 carbon atoms, more preferably a 1-substituted alkoxymethyl group having 4 to 18 carbon atoms, further preferably a 1-substituted alkoxymethyl group having 6 to 16 carbon atoms. Specific examples of the 1-substituted alkoxymethyl group can include, but are not limited to, a 1-cyclopentylmethoxymethyl group, a 1-cyclopentylethoxymethyl group, a 1-cyclohexylmethoxymethyl group, a 1-cyclohexylethoxymethyl group, a 1-cyclooctylmethoxymethyl group, and a 1-adamantylmethoxymethyl group.

The cyclic ether group having 2 to 20 carbon atoms and having the property of being dissociated by an acid is preferably a cyclic ether group having 4 to 18 carbon atoms, more preferably a cyclic ether group having 6 to 16 carbon atoms. Specific examples of the cyclic ether group can include, but are not limited to, a tetrahydropyranyl group, a tetrahydrofuranyl group, a tetrahydrothiopyranyl group, a tetrahydrothiofuranyl group, a 4-methoxytetrahydropyranyl group, and a 4-methoxytetrahydrothiopyranyl group.

The alkoxycarbonyl group having 2 to 20 carbon atoms and having the property of being dissociated by an acid is preferably an alkoxycarbonyl group having 4 to 18 carbon atoms, more preferably an alkoxycarbonyl group having 6 to 16 carbon atoms. Specific examples of the alkoxycarbonyl group can include, but are not limited to, a methoxycarbonyl group, an ethoxycarbonyl group, a n-propoxycarbonyl group, an isopropoxycarbonyl group, a n-butoxycarbonyl group, a tert-butoxycarbonyl group, and a group represented by the following formula (3) wherein n=0.

The alkoxycarbonylalkyl group and having the property of being dissociated by an acid is preferably an alkoxycarbonylalkyl group having 3 to 20 carbon atoms, more preferably an alkoxycarbonylalkyl group having 4 to 18 carbon atoms, further preferably an alkoxycarbonylalkyl group having 6 to 16 carbon atoms. Specific examples of the alkoxycarbonylalkyl group can include, but are not limited to, a methoxycarbonylmethyl group, an ethoxycarbonylmethyl group, a n-propoxycarbonylmethyl group, an isopropoxycarbonylmethyl group, a n-butoxycarbonylmethyl group, and a group represented by the following formula (3) wherein n=1 to 4.

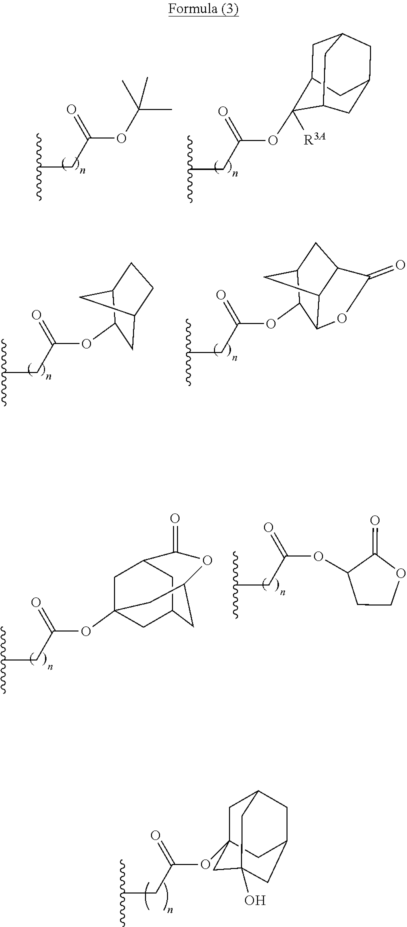

In formula (3). R^{3A} is a hydrogen atom, or a straight or branched alkyl group having 1 to 4 carbon atoms, and n is an integer of 0 to 4.

In formula (P-0C), R¹² to R¹⁵ are each independently an alkyl group having 2 to 20 carbon atoms, the group represented by formula (TS-0), the group represented by formula (TS-1), or a group represented by formula (P-0C-1). The alkyl group having 2 to 20 carbon atoms is preferably an alkyl group having 2 to 10 carbon atoms, and examples thereof include an ethyl group, a propyl group, an isopropyl group, a butyl group, an isobutyl group, a t-butyl group, and a decyl group.

In formula (P-0C-1), each R²¹ is independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, a cyano group, a nitro group, a heterocyclic group, a halogen atom, a carboxyl group, an alkylsilyl group having 1 to 20 carbon atoms; or a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkcyl group, each of which has the property of being dissociated by an acid. These groups can be the same as R¹⁶ to R¹⁹ in formula (P-0C).

Herein, in formula (P-0C), at least one of R¹² to R¹⁹ is the group represented by formula (TS-0) or the group represented by formula (TS-1). In formula (P-0C), m⁷ to m¹⁰ are each independently an integer of 1 to 4, preferably an integer of 1 to 3. In formula (P-0C-1), p⁷ is an integer of 0 to 5, preferably an integer of 0 to 3.

Examples of the compound represented by formula (P-0C) include any compound disclosed in Japanese Patent Laid-Open No. 2009-173623 and Japanese Patent Laid-Open No. 2009-173625, in which a hydroxy group (—OH) is substituted with a group represented by —OR^{TS}, and specific examples include the following compounds. Herein, the compound represented by formula (P-0C) is not limited to these specific compounds.

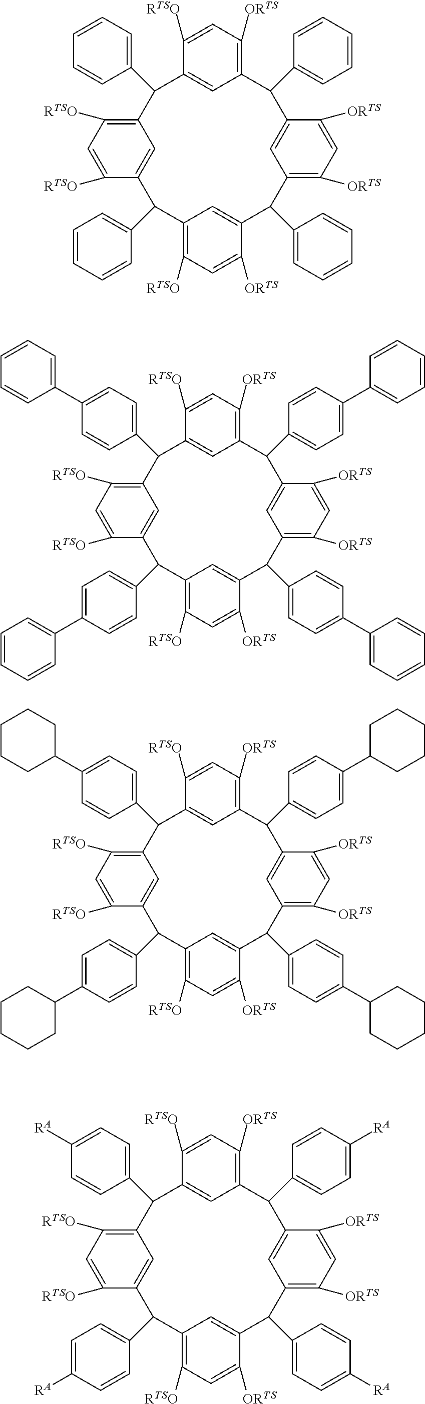

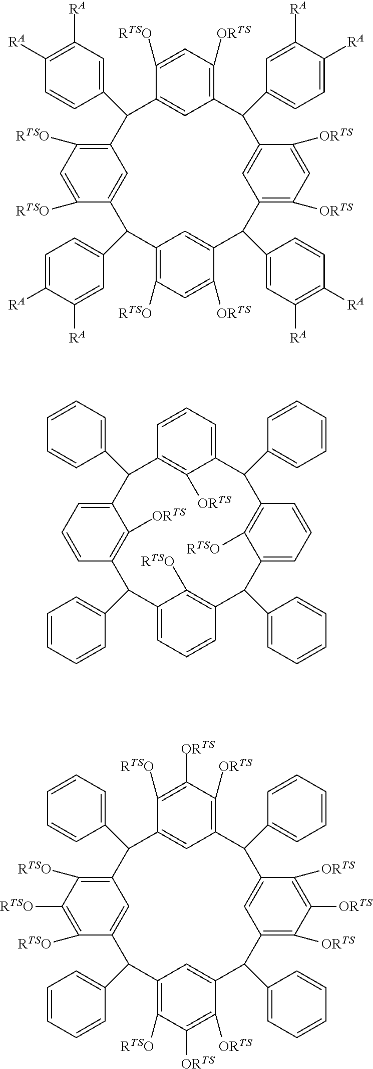

In the formulae, R^{A} is a fluorine atom, a chlorine atom, a bromine atom, an iodine atom, a hydroxy group, a cyano group, a nitro group, an amino group, a thiol group, a heterocyclic group, a straight aliphatic hydrocarbon group having 1 to 20 carbon atoms, a branched aliphatic hydrocarbon group having 3 to 20 carbon atoms, a cyclic aliphatic hydrocarbon group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 20 carbon atoms, an alkoxycarbonyl group having 2 to 20 carbon atoms, an alkyloyloxy group having 1 to 20 carbon atoms, an aryloyloxy group having 7 to 30 carbon atoms, or an alkylsilyl group having 1 to 20 carbon atoms, and OR^{TS} has the same meaning as in formula (P-0). Examples of the straight aliphatic hydrocarbon group having 1 to 20 carbon atoms include a methyl group, an ethyl group, a propyl group, a butyl group, an octyl group, a nonyl group, a decyl group, and a dodecyl group.

The compound represented by formula (P-0) is preferably, for example, a compound represented by the following formula (P-1).

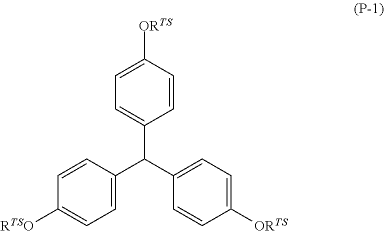

In formula (P-1), OR^{TS} has the same meaning as in formula (P-0).

Examples of the compound represented by formula (P-1) include the following compounds. Herein, the compound represented by formula (P-1) is not limited to these specific compounds.

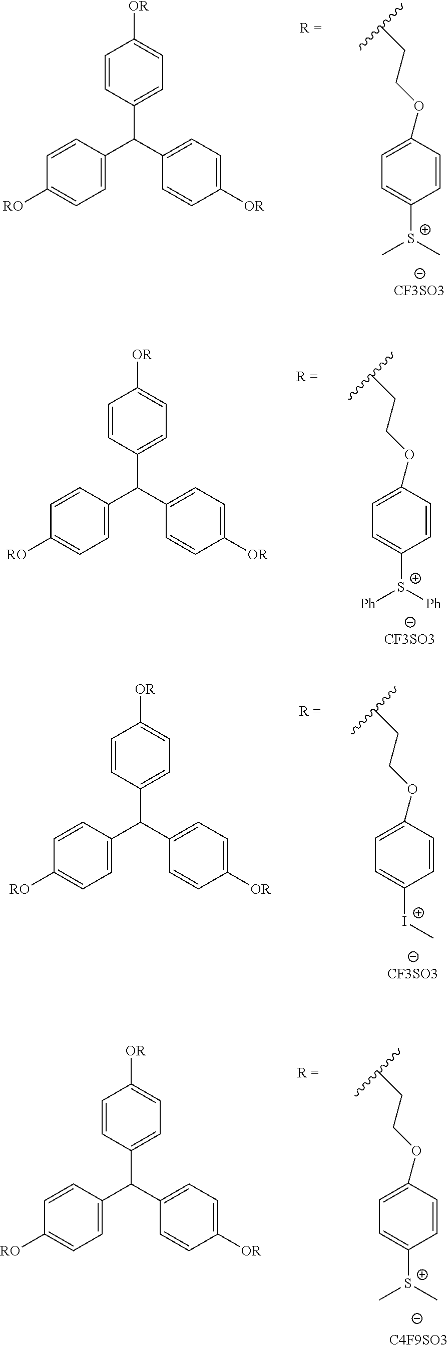

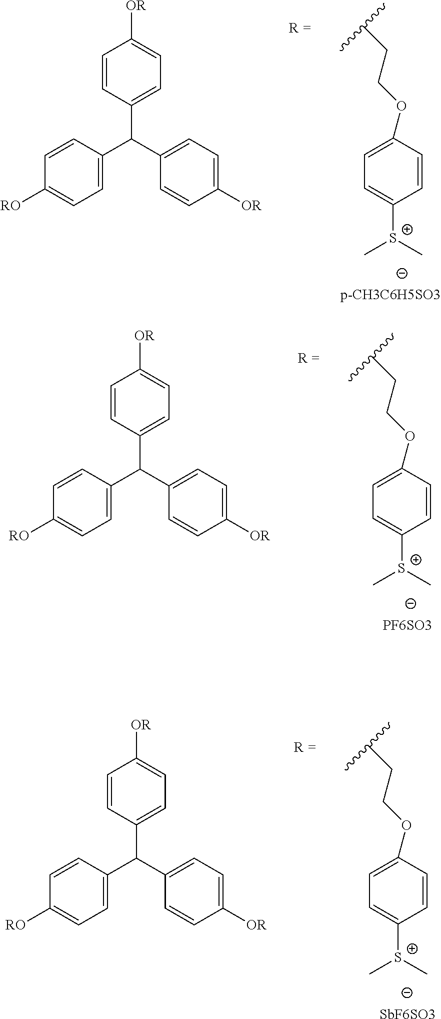

[Method for Producing Compound]

A method for producing the compound according to the present embodiment includes a step (hereinafter, also referred to as “condensation step”) of condensing a compound represented by the following formula (P-0′), and a compound represented by the following formula (TS-0) or a compound represented by the following formula (TS-1′) to thereby obtain a condensate, and a step (hereinafter, also referred to as “alkylating step”) of reacting the condensate, a salt having an anion containing fluorine or iodine, and an alkylating agent.

Herein, unless otherwise defined, the “alkylating” represents alkylating or arylating, the “alkylating agent” represents an alkylating agent or an arylating agent, and the “alkylating step” represents an alkylating step or an arylating step.

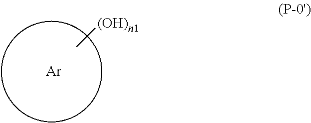

In formula (P-0′), Ar and n¹ have the same meanings as in formula (P-0).

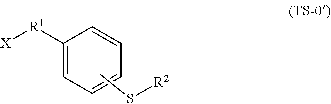

In formula (TS-0′), X is a halogen atom, and R¹ and R² have the same meanings as in formula (TS-0).

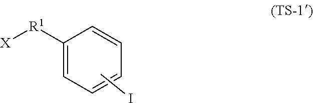

In formula (TS-1′), X is a halogen atom and R¹ has the same meaning as in formula (TS-1).

According to the above method, the compound according to the present embodiment can be efficiently produced. Examples of the halogen atom in formula (TS-0′) and formula (TS-1′) include a fluorine atom, a chlorine atom, a bromine atom, and an iodine atom.

(Condensation Step)

In the present step, the compound represented by formula (P-0′), and the compound represented by formula (TS-0′) or the compound represented by formula (TS-1′) are condensed to thereby obtain a condensate. The compound represented by formula (TS-0′) or the compound represented by formula (TS-1′) can be obtained by, for example, reacting the compound of formula (TS-0′) or formula (TS-1′), in which an XR¹ group is a hydroxy group, with X—R¹—X. The condensation reaction of the compound represented by formula (P-0′) and the compound represented by formula (TS-0′) or the compound represented by formula (TS-1′) can be performed by, for example, a method in which a condensation reaction is made in the presence of a strong acid.

(Alkylating Step)

In the present step, the condensate obtained in the condensation step, a salt having an anion containing fluorine or iodine, and an alkylating agent are reacted. The reaction can be performed by, for example, a method including a reaction with an alkali metal salt of an anion or an acid represented by H⁺X⁻.

[First Composition]

A first composition according to the present embodiment includes the compound according to the present embodiment. The first composition according to the present embodiment can be, for example, a material for lithography or a material composition for lithography.

(Material for Lithography)

A material for lithography according to the present embodiment includes the compound according to the present embodiment. The material for lithography according to the present embodiment is a material usable in lithography techniques, is not particularly limited as long as it includes the compound according to the present embodiment, and, for example, can be taken with a solvent or the like and thus used as a material composition for lithography or also used in, for example, a resist application (namely, resist composition).

The material for lithography according to the present embodiment includes the compound according to the present embodiment, and thus has high sensitivity and provides high resolution and high flatness. The material for lithography according to the present embodiment can include no solvent.

(Material Composition for Lithography)

A material composition for lithography according to the present embodiment includes the material for lithography according to the present embodiment, and a solvent. The material composition for lithography has high sensitivity and provides high resolution and high flatness, and thus can impart a favorable resist pattern shape. For example, a resist film can be formed from the material composition for lithography.

<Physical Properties and the Like of Material Composition for Lithography>

The material for lithography of the present embodiment can be used in a resist application, as described above, and can form an amorphous film by a known method such as spin coating. A positive resist pattern and a negative resist pattern can be separately formed depending on the type of a developer used. Hereinafter, a case will be described where the material composition for lithography including the material for lithography of the present embodiment is used in a resist application (as a resist composition).

In a case where the material composition for lithography of the present embodiment corresponds to a positive resist pattern, the rate of dissolution at 23° C. of an amorphous film formed by spin coating with the material composition for lithography of the present embodiment, in a developer, is preferably 5 Å/sec or less, more preferably 0.05 to 5 Å/sec, further preferably 0.0005 to 5 Å/sec. When the rate of dissolution is 5 Å/sec or less, a resist insoluble in the developer can be provided. When the rate of dissolution is 0.0005 Å/sec or more, resolution performance may also be enhanced. It is presumed that the reason is because the change in solubility before and after exposure of the compound according to the present embodiment leads to an increase in contrast at the interface between an exposed region soluble in the developer and an unexposed region insoluble in the developer. The effects of reducing line edge roughness and of reducing defects are also exerted.

In a case where the material composition for lithography of the present embodiment corresponds to a negative resist pattern, the rate of dissolution at 23° C. of an amorphous film formed by spin coating with the material composition for lithography of the present embodiment, in a developer, is preferably 10 Å/sec or more. When the rate of dissolution is 10 Å/sec or more, the material composition is easily soluble in the developer and is much more suitable for a resist. When the rate of dissolution is 10 Å/sec or more, resolution performance may also be enhanced. It is presumed that the reason is because a micro surface portion of the compound according to the present embodiment is dissolved to result in a reduction in line edge roughness. The effect of reducing defects is also exerted. The rate of dissolution can be determined by dipping the amorphous film in the developer at 23° C. for a predetermined time, and measuring the film thickness before and after the dipping, visually or by a known method such as an ellipsometer or a QCM method.

In a case where the material composition for lithography of the present embodiment corresponds to a positive resist pattern, the rate of dissolution at 23° C. of a region of an amorphous film formed by spin coating with the material composition for lithography of the present embodiment, in a developer, the region being exposed by radiation from, for example, a KrF excimer laser, extreme ultraviolet light, electron beam or X-ray, is preferably 10 Å/sec or more. When the rate of dissolution is 10 Å/sec or more, the material composition is easily soluble in the developer and is much more suitable for a resist. When the rate of dissolution is 10 Å/sec or more, resolution performance may also be enhanced. It is presumed that the reason is because a micro surface portion of the compound according to the present embodiment is dissolved to result in a reduction in line edge roughness. The effect of reducing defects is also exerted.

In a case where the material composition for lithography of the present embodiment corresponds to a negative resist pattern, the rate of dissolution at 23° C. of a region of an amorphous film formed by spin coating with the material composition for lithography of the present embodiment, in a developer, the region being exposed by radiation from, for example, a KrF excimer laser, extreme ultraviolet light, electron beam or X-ray, is preferably 5 Å/sec or less, more preferably 0.05 to 5 Å/sec, further preferably 0.0005 to 5 Å/sec. When the rate of dissolution is 5 Å/sec or less, a resist insoluble in the developer can be provided. When the rate of dissolution is 0.0005 Å/sec or more, resolution performance may also be enhanced. It is presumed that the reason is because the change in solubility before and after exposure of the compound according to the present embodiment leads to an increase in contrast at the interface between an unexposed region soluble in the developer and an exposed region insoluble in the developer. The effects of reducing line edge roughness and of reducing defects are also exerted.

<Other Component in Material Composition for Lithography>

The material composition for lithography of the present embodiment includes the compound according to the present embodiment, as a solid component. The material composition for lithography of the present embodiment further includes a solvent, in addition to the compound according to the present embodiment.

The solvent for use in the material composition for lithography of the present embodiment is not particularly limited, and examples thereof can include ethylene glycol monoalkyl ether acetates such as ethylene glycol monomethyl ether acetate, ethylene glycol monoethyl ether acetate, ethylene glycol mono-n-propyl ether acetate and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate, propylene glycol monoethyl ether acetate(PGMEA), propylene glycol mono-n-propyl ether acetate and propylene glycol mono-n-butyl ether acetate; propylene glycol monoalkyl ethers such as propylene glycol monomethyl ether(PGME) and propylene glycol monoethyl ether: lactic acid esters such as methyl lactate, ethyl lactate, n-propyl lactate, n-butyl lactate and n-amyl lactate; aliphatic carboxylic acid esters such as methyl acetate, ethyl acetate, n-propyl acetate, n-butyl acetate, n-amyl acetate, n-hexyl acetate, methyl propionate and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutyl acetate, 3-methyl-3-methoxybutyl acetate, butyl 3-methoxy-3-methylpropionate, butyl 3-methoxy-3-methylbutyrate, methyl acetoacetate, methyl pyruvate and ethyl pyruvate; aromatic hydrocarbons such as toluene and xylene; ketones such as methyl ethyl ketone, 2-heptanone, 3-heptanone, 4-heptanone, cyclopentanone (CPN) and cyclohexanone (CHN); amides such as N,N-dimethylformamide, N-methylacetamide, N,N-dimethylacetamide and N-methylpyrrolidone: and lactones such as γ-lactone. These solvents can be used singly or in combinations of two or more kinds thereof.

The solvent for use in the material composition for lithography of the present embodiment is preferably a safe solvent, more preferably at least one selected from PGMEA, PGME, CHN, CPN, 2-heptanone, anisole, butyl acetate, ethyl propionate and ethyl lactate, further preferably at least one selected from PGMEA, PGME and CHN.

The relationship between the amount of the solid component and the amount of the solvent in the material composition for lithography of the present embodiment is not particularly limited, and is preferably a relationship between 1 to 80 mass % of the solid component and 20 to 99 mass % of the solvent, more preferably 1 to 50 mass % of the solid component and 50 to 99 mass % of the solvent, further preferably 2 to 40 mass % of the solid component and 60 to 98 mass % of the solvent, particularly preferably 2 to 10 mass % of the solid component and 90 to 98 mass % of the solvent, based on 100 mass % of the total mass of the solid component and the solvent.

The material composition for lithography of the present embodiment may include at least one selected from the group consisting of an acid generating agent (C), an acid crosslinking agent (G), an acid diffusion controlling agent (E) and other component (F), as other solid component.

The content of the compound according to the present embodiment in the material composition for lithography of the present embodiment is not particularly limited, and is preferably 50 to 99.4 mass %, more preferably 55 to 90 mass %, further preferably 60 to 80 mass %, particularly preferably 60 to 70 mass %, based on the mass of the total solid component (the sum of the compound according to the present embodiment, and the solid component(s) optionally used, for example, the acid generating agent (C), the acid crosslinking agent (G), the acid diffusion controlling agent (E) and other component (F), the same applies to the following). The above content results in a further enhancement in resolution and a further reduction in line edge roughness (LER).

<Acid Generating Agent (C)>

The material composition for lithography of the present embodiment preferably includes at least one acid generating agent (C) which directly or indirectly generates an acid by irradiation with any radiation selected from visible light, ultraviolet light, excimer laser, electron beam, extreme ultraviolet light (EUV), X-ray and ion beam.

In this case, the content of the acid generating agent (C) in the material composition for lithography of the present embodiment is preferably 0.001 to 49 mass %, more preferably 1 to 40 mass %, further preferably 3 to 30 mass %, particularly preferably 10 to 25 mass %, based on the mass of the total solid component. The acid generating agent (C) is used in the range of the content, and thus higher sensitivity is achieved and a pattern profile lower in edge roughness is obtained.

The acid generation method is not limited as long as an acid is generated in the system in the material composition for lithography of the present embodiment. If excimer laser is used instead of ultraviolet light such as g-ray or i-ray, finer processing can be made, and if electron beam, extreme ultraviolet light, X-ray or ion beam is used as a high energy line, further finer processing can be made.

The acid generating agent (C) is not particularly limited, and examples thereof include any compound disclosed in International Publication No. WO2017/033943. The acid generating agent (C) is preferably an acid generating agent having an aromatic ring, more preferably an acid generating agent having an sulfonic acid ion having an aryl group, particularly preferably diphenyltrimethylphenylsulfonium p-toluenesulfonate, triphenylsulfonium p-toluenesulfonate, triphenylsulfonium trifluoromethanesulfonate, or triphenylsulfonium nonafluoromethanesulfonate. Use of the acid generating agent can result in a reduction in line edge roughness.

The material composition for lithography of the present embodiment preferably further includes an optically active diazonaphthoquinone compound as the acid generating agent. The optically active diazonaphthoquinone compound is any diazonaphthoquinone substance including optically active polymeric or non-polymeric diazonaphthoquinone compound, and is not particularly limited as long as it can be generally used as a photosensitive component in a positive resist composition, and one or more thereof can be arbitrarily selected and used. In particular, an optically active non-polymeric diazonaphthoquinone compound is preferable, a low molecular compound having a molecular weight of 1500 or less is more preferable, a low molecular compound having a molecular weight of 1200 or less is further preferable, and a low molecular compound having a molecular weight of 1000 or less is particularly preferable, from the viewpoints of low roughness and the solubility. Preferable specific examples of the optically active non-polymeric diazonaphthoquinone compound include any optically active non-polymeric diazonaphthoquinone compound disclosed in International Publication No. WO2016/158881. The acid generating agent (C) can be used singly or in combinations of two or more kinds thereof.

<Acid Crosslinking Agent (G)>

In a case where the material composition for lithography of the present embodiment is used as a negative resist material or is used as an additive for an increase in strength of a pattern even in the case of a positive resist material, it preferably includes at least one acid crosslinking agent (G). The acid crosslinking agent (G) is a compound which can intramolecularly or intermolecularly crosslink the compound according to the present embodiment in the presence of an acid generated from the acid generating agent (C). The acid crosslinking agent (G) is not particularly limited, and examples thereof can include a compound having at least one crosslinkable group which can crosslink the compound according to the present embodiment.

Specific examples of the crosslinkable group can include, but not particularly limited, (i) hydroxyalkyl groups such as hydroxy (alkyl group having 1 to 6 carbon atoms), alkoxy having 1 to 6 carbon atoms (alkyl group having 1 to 6 carbon atoms), and acetoxy (alkyl group having 1 to 6 carbon atoms), or groups derived therefrom: (ii) carbonyl groups such as a formyl group and carboxy (alkyl group having 1 to 6 carbon atoms), or groups derived therefrom; (iii) nitrogen-containing group-containing groups such as a dimethylaminomethyl group, a diethylaminomethyl group, a dimethylolaminomethyl group, a diethylolaminomethyl group and a morpholinomethyl group; (iv) glycidyl group-containing groups such as a glycidyl ether group, a glycidyl ester group and a glycidyl amino group; (v) groups derived from aromatic groups of allyloxy having 1 to 6 carbon atoms (alkyl group having 1 to 6 carbon atoms), aralkyloxy having 1 to 6 carbon atoms (alkyl group having 1 to 6 carbon atoms), and the like, such as a benzyloxymethyl group and a benzoyloxymethyl group; and (vi) polymerizable multiple bond-containing groups such as a vinyl group and an isopropenyl group. The crosslinkable group in the acid crosslinking agent (G) is preferably a hydroxyalkyl group or an alkoxyalkyl group, particularly preferably an alkoxymethyl group.

Examples of the acid crosslinking agent (G) having the crosslinkable group can include, but not particularly limited, (i) methylol group-containing compounds such as a methylol group-containing melamine compound, a methylol group-containing benzoguanamine compound, a methylol group-containing urea compound, a methylol group-containing glycoluryl compound and a methylol group-containing phenolic compound; (ii) alkoxyalkyl group-containing compounds such as an alkoxyalkyl group-containing melamine compound, an alkoxyalkyl group-containing benzoguanamine compound, an alkoxyalkyl group-containing urea compound, an alkoxyalkyl group-containing glycoluryl compound and an alkoxyalkyl group-containing phenolic compound: (iii) carboxymethyl group-containing compounds such as a carboxymethyl group-containing melamine compound, a carboxymethyl group-containing benzoguanamine compound, a carboxymethyl group-containing urea compound, a carboxymethyl group-containing glycoluryl compound and a carboxymethyl group-containing phenolic compound: (iv) epoxy compounds such as a bisphenol A-based epoxy compound, a bisphenol F-based epoxy compound, a bisphenol S-based epoxy compound, a novolac resin-based epoxy compound, a resol resin-based epoxy compound and a poly(hydroxystyrene)-based epoxy compound.

A compound having a phenolic hydroxy group, and a compound and a resin each obtained by introducing the crosslinkable group into an acidic functional group in an alkali-soluble resin to thereby impart crosslinkability can be further used as the acid crosslinking agent (G). The rate of introduction of the crosslinkable group is not particularly limited, and is regulated so as to be, for example, 5 to 100 mol %, preferably 10 to 60 mol %, further preferably 15 to 40 mol %, relative to the entire acidic functional group in the compound having a phenolic hydroxy group and the alkali-soluble resin. The above range is preferable because of allowing the crosslinking reaction to sufficiently occur and allowing a reduction in rate of the residual film, a swelling phenomenon and meandering of a pattern, and the like to be avoided.

The acid crosslinking agent (G) in the material composition for lithography of the present embodiment is preferably an alkoxyalkylating urea compound or a resin thereof, or an alkoxyalkylating glycoluryl compound or a resin thereof (acid crosslinking agent (G1)), a phenol derivative having 1 to 6 benzene rings in its molecule and having two or more hydroxyalkyl groups or alkoxyalkyl groups in its molecule, in which the hydroxyalkyl groups or alkoxyalkyl groups are bound to any benzene ring described above (acid crosslinking agent (G2)), or a compound having at least one α-hydroxyisopropyl group (acid crosslinking agent (G3)). Examples include any compound disclosed in International Publication No. WO2017/033943.

The content of the acid crosslinking agent (G) in the material composition for lithography of the present embodiment is preferably 0.5 to 49 mass %, more preferably 0.5 to 40 mass %, further preferably 1 to 30 mass %, particularly preferably 2 to 20 mass %, based on the mass of the total solid component. The content ratio of the acid crosslinking agent (G) is preferably 0.5 mass % or more because of enabling the effect of suppressing solubility of a resist film in an alkaline developing solution to be enhanced, and enabling a reduction in rate of the residual film, and the occurrence of swelling and meandering of a pattern to be suppressed, and on the other hand, the content ratio is preferably 49 mass % or less because of enabling deterioration in heat resistance of a resist to be suppressed.

The content of at least one selected from the acid crosslinking agent (G1), the acid crosslinking agent (G2) and the acid crosslinking agent (G3) in the acid crosslinking agent (G) is also not particularly limited, and can fall within various ranges depending on the type and the like of the substrate for use in resist pattern formation.

<Acid Diffusion Controlling Agent (E)>

The material composition for lithography of the present embodiment may include an acid diffusion controlling agent (E) which has the effect of controlling diffusion of an acid generated from the acid generating agent by irradiation with radiation, in a resist film, to thereby inhibit an undesirable chemical reaction in an unexposed region. The acid diffusion controlling agent (E) is used to result in an enhancement in preservation stability of the material composition for lithography. Additionally, not only the resolution is further enhanced, but also a resist pattern can be inhibited from being changed in line width due to the variations in post exposure delay before irradiation with radiation and post exposure delay after irradiation with radiation, and is extremely excellent in process stability.

The acid diffusion controlling agent (E) is not particularly limited, and examples thereof include radiation-degradable basic compounds such as a nitrogen atom-containing basic compound, a basic sulfonium compound and a basic iodonium compound. Examples of the acid diffusion controlling agent (E) include any compound disclosed in International Publication No. WO2017/033943. The acid diffusion controlling agent (E) can be used singly or in combinations of two or more kinds thereof.

The content of the acid diffusion controlling agent (E) is preferably 0.001 to 49 mass %, more preferably 0.01 to 10 mass %, further preferably 0.01 to 5 mass %, particularly preferably 0.01 to 3 mass %, based on the mass of the total solid component. When the content of the acid diffusion controlling agent (E) is in the above range, a reduction in resolution, and degradation of a pattern shape, dimensional faithfulness and the like can be further suppressed. Furthermore, even if the post exposure delay after irradiation with radiation from irradiation with electron beam is increased, the shape of the upper layer portion of a pattern is not degraded. When the content of the acid diffusion controlling agent (E) is 10 mass % or less, sensitivity, developability in an unexposed region, and the like can be prevented from being deteriorated. The acid diffusion controlling agent is used to not only result in an enhancement in preservation stability of the material composition for lithography and an enhancement in resolution, but also enable a resist pattern to be inhibited from being changed in line width due to the variations in post exposure delay before irradiation with radiation and post exposure delay after irradiation with radiation, and to be extremely excellent in process stability.

(Other Component (F))

One or more of various additives such as a dissolution promoting agent, a dissolution controlling agent, a sensitizing agent, a surfactant, and an organic carboxylic acid or an oxo acid of phosphorus, or any derivative thereof can be, if necessary, added as other component (F) to the material composition for lithography of the present embodiment as long as the objects of the present embodiment are not impaired. Examples of such other component (F) include any compound disclosed in International Publication No. WO2017/033943.

The total content of such other component (F) is preferably 0 to 49 mass %, more preferably 0 to 5 mass %, further preferably 0 to 1 mass %, particularly preferably 0 mass %, based on the mass of the total solid component.

The contents of the compound according to the present embodiment, the acid generating agent (C), the acid diffusion controlling agent (E) and such other component (F) (the compound according to the present embodiment/acid generating agent (C)/acid diffusion controlling agent (E)/other component (F)) in the material composition for lithography of the present embodiment are preferably 50 to 99.4/0.001 to 49/0.001 to 49/0 to 49, more preferably 55 to 90/1 to 40/0.01 to 10/0 to 5, further preferably 60 to 80/3 to 30/0.01 to 5/0 to 1, particularly preferably 60 to 70/10 to 25/0.01 to 3/0, as expressed by mass % on the solid content basis.

The content ratio among the respective components is selected from such various ranges so that the sum of the components is 100 mass %. The above content ratio allows performances such as sensitivity, resolution, and developability to be further excellent.

The method for preparing the material composition for lithography of the present embodiment is not particularly limited, and examples thereof include a method including dissolving the respective components in the solvent in use to thereby provide a uniform solution, and thereafter, if necessary, filtering the solution by, for example, a filter having a pore size of about 0.2 μm.

The material composition for lithography of the present embodiment can include a resin as long as the objects of the present invention are not impaired. The resin is not particularly limited, and examples thereof include a novolac resin, polyvinylphenols, polyacrylic acid, polyvinylalcohol, a styrene-maleic anhydride resin, and a polymer containing acrylic acid, vinylalcohol or vinylphenol as a monomer unit, or any derivative thereof. The content of the resin is not particularly limited, is appropriately regulated depending on the type of the compound according to the present embodiment, here used, and is preferably 30 parts by mass or less, more preferably 10 parts by mass or less, further preferably 5 parts by mass or less, particularly preferably 0 parts by mass, per 100 parts by mass of the compound.

[Method for Forming Pattern]

In a case where a pattern is formed on a substrate by use of a material for lithography, for example, a method for forming a pattern can be used which includes a film formation step of forming a film on a substrate by use of the material for lithography according to the present embodiment or a composition including the material (hereinafter, these may be sometimes collectively referred to as “material or the like for lithography”), an exposure step of exposing the film, and a development step of developing the film exposed in the exposure step, to thereby form a pattern.

For example, in a case where the material or the like for lithography of the present embodiment is used to form a resist pattern, a method for forming a pattern (resist pattern) is not particularly limited, and examples of a suitable method include a method including a film formation step of coating a substrate with a resist composition including the above material or the like for lithography to thereby form a film (resist film), an exposure step of exposing the film (resist film) formed, and a development step of developing the film (resist film) exposed in the exposure step to thereby form a pattern (resist pattern). The resist pattern in the present embodiment can also be formed as an upper layer resist in a multi-layer process.

Examples of a specific method for forming the resist pattern include, but not particularly limited, the following method. First, a conventionally known substrate is coated with the resist composition by a coating procedure such as rotation coating, cast coating, or roll coating to thereby form a resist film. The conventionally known substrate is not particularly limited, and examples thereof include a substrate for electronic components, and such a substrate on which a predetermined wiring pattern is formed. More specific examples include, but not particularly limited, a silicon wafer, substrates made of metals such as copper, chromium, iron, and aluminum, and a glass substrate. Examples of the material for the wiring pattern include, but not particularly limited, copper, aluminum, nickel, and gold. An inorganic film or an organic film may be, if necessary, provided on the above substrate. Examples of the inorganic film include, but not particularly limited, an inorganic antireflective film (inorganic BARC). Examples of the organic film include, but not particularly limited, an organic antireflective film (organic BARC). A surface treatment with hexamethylene disilazane or the like may also be performed.

Next, the substrate coated is, if necessary, heated. The heating condition, while varied depending on the compositional profile of the resist composition, and the like, is preferably 20 to 250° C., more preferably 20 to 150° C. Such heating is preferable because of sometimes resulting in an enhancement in close contact of the resist with the substrate. Next, the resist film is exposed to any radiation selected from the group consisting of visible light, ultraviolet light, excimer laser, electron beam, extreme ultraviolet light (EUV), X-ray and ion beam, to thereby provide a desired pattern. The exposure conditions and the like are appropriately selected depending on the compositional profile of compounding in the resist composition, and the like.

In the method for forming a resist pattern of the present embodiment, heating is preferably performed after irradiation with radiation in order to stably form a high-accuracy and fine pattern in exposure. The heating condition, while varied depending on the compositional profile of compounding in the resist composition, and the like, is preferably 20 to 250° C., more preferably 20 to 150° C.

Next, the resist film exposed is developed by a developer to thereby form a predetermined resist pattern. The developer here selected is preferably a solvent having a solubility parameter (SP value) closer to that of the compound according to the present embodiment, here used, and a polar solvent or a hydrocarbon-based solvent, such as a ketone-based solvent, an ester-based solvent, an alcohol-based solvent, an amide-based solvent or an ether-based solvent, or an aqueous alkaline solution can be used. While a positive resist pattern or a negative resist pattern can be separately formed depending on the type of the developer, in general, a negative resist pattern is obtained in the case of a polar solvent or a hydrocarbon-based solvent, such as a ketone-based solvent, an ester-based solvent, an alcohol-based solvent, an amide-based solvent or an ether-based solvent, and a positive resist pattern is obtained in the case of an aqueous alkaline solution. Examples of the ketone-based solvent, the ester-based solvent, the alcohol-based solvent, the amide-based solvent, the ether-based solvent, hydrocarbon-based solvent, and the aqueous alkaline solution include any disclosed in International Publication No. WO2017/033943.

A plurality of such solvents may be mixed, or such a solvent may be mixed with a solvent other than such solvents, or water, and then used, as long as performance is exhibited. The water content in the entire developer is preferably less than 70 mass %, further preferably less than 50 mass %, more preferably less than 30 mass %, further preferably less than 10 mass %, and no moisture is particularly preferably substantially contained, in order to fully exert the effects of the present invention. In other words, the content of the organic solvent in the developer is not particularly limited, and is preferably 30 mass % or more and 100 mass % or less, further preferably 50 mass % or more and 100 mass % or less, more preferably 70 mass % or more and 100 mass % or less, further preferably 90 mass % or more and 100 mass % or less, particularly preferably 95 mass % or more and 100 mass % or less, based on the amount of the entire developer.

In particular, the developer is preferably a developer containing at least one solvent selected from the ketone-based solvent, the ester-based solvent, the alcohol-based solvent, the amide-based solvent and the ether-based solvent because resist performances such as resolution performance, roughness, and the like of a resist pattern are improved.

The vapor pressure at 20° C. of the developer is not particularly limited, and is, for example, preferably 5 kPa or less, further preferably 3 kPa or less, particularly preferably 2 kPa or less. The vapor pressure of the developer is 5 kPa or less, and thus the developer is inhibited from being evaporated on the substrate or in a development cup and temperature uniformity in a wafer surface is enhanced, resulting in an improvement in dimension uniformity in a wafer surface. Examples of the developer having such a vapor pressure include any developer disclosed in International Publication No. WO2017/033943.

A proper amount of a surfactant can be, if necessary, added to the developer. The surfactant is not particularly limited, and, for example, an ionic or non-ionic fluorine-based or silicon-based surfactant can be used. Examples of such a fluorine or silicon-based surfactant can include respective surfactants described in Japanese Patent Laid-Open No. 62-36663, Japanese Patent Laid-Open No. 61-226746, Japanese Patent Laid-Open No. 61-226745, Japanese Patent Laid-Open No. 62-170950, Japanese Patent Laid-Open No. 63-34540, Japanese Patent Laid-Open No. 7-230165, Japanese Patent Laid-Open No. 8-62834, Japanese Patent Laid-Open No. 9-54432, Japanese Patent Laid-Open No. 9-5988, and U.S. Pat. Nos. 5,405,720, 5,360,692, 5,529,881, 5,296,330, 5,436,098, 5,576,143, 5,294,511 and 5,824,451, and an non-ionic surfactant is preferable. The non-ionic surfactant is not particularly limited, and a fluorine-based surfactant or a silicon-based surfactant is further preferably used.

The amount of the surfactant used is usually 0.001 to 5 mass %, preferably 0.005 to 2 mass %, further preferably 0.01 to 0.5 mass %, based on the amount of the entire developer.

The development method here applied can be, for example, a method (dipping method) including dipping the substrate in a tank filled with the developer, for a certain time, a method (paddle method) including raising the developer on the substrate surface by surface tension and leaving it to still stand for a certain time for development, a method (spraying method) including spraying the developer on the substrate surface, or a method (dynamic dispense method) including continuously discharging the developer onto the substrate rotating at a certain speed, with scanning of a developer discharge nozzle at a certain speed. The time for pattern development is not particularly limited, and is preferably 10 seconds to 90 seconds.

After the development step, a step of stopping development under replacement with other solvent may be performed.

A step of washing with a rinsing liquid including an organic solvent, after development, is preferably included.

The rinsing liquid for use in a rinsing step after development is not particularly limited and a solution including a common organic solvent, or water can be used, as long as a resist pattern obtained by curing by crosslinking is not dissolved. The rinsing liquid here used is preferably a rinsing liquid containing at least one organic solvent selected from a hydrocarbon-based solvent, a ketone-based solvent, an ester-based solvent, an alcohol-based solvent, an amide-based solvent and an ether-based solvent. More preferably, after development, a step of washing with a rinsing liquid containing at least one organic solvent selected from the group consisting of a ketone-based solvent, an ester-based solvent, an alcohol-based solvent, and an amide-based solvent is performed. Further preferably, after development, a step of washing with a rinsing liquid containing an alcohol-based solvent or an ester-based solvent is performed. Still more preferably, after development, a step of washing with a rinsing liquid containing a monohydric alcohol is performed. Particularly preferably, after development, a step of washing with a rinsing liquid containing a monohydric alcohol having 5 or more carbon atoms is performed. The time for pattern rinsing is not particularly limited, and is preferably 10 seconds to 90 seconds.

Examples of the monohydric alcohol for use in the rinsing step after development include, but not particularly limited, straight, branched, and cyclic monohydric alcohols, specifically, 1-butanol, 2-butanol, 3-methyl-1-butanol, tert-butyl alcohol, 1-pentanol, 2-pentanol, 1-hexanol, 4-methyl-2-pentanol, 1-heptanol, 1-octanol, 2-hexanol, cyclopentanol, 2-heptanol, 2-octanol, 3-hexanol, 3-heptanol, 3-octanol, 4-octanol, or the like can be used, and a particularly preferable monohydric alcohol having 5 or more carbon atoms can be, for example, 1-hexanol, 2-hexanol, 4-methyl-2-pentanol, 1-pentanol, or 3-methyl-1-butanol.

A plurality of such components may be mixed, or such a component may be mixed with an organic solvent other than the above solvents.

The water content in the rinsing liquid is not particularly limited, and is preferably 10 mass % or less, more preferably 5 mass % or less, particularly preferably 3 mass % or less. The water content is 10 mass % or less, thereby enabling more favorable development characteristics to be obtained.

The vapor pressure at 20° C. of the rinsing liquid used after development is preferably 0.05 kPa or more and 5 kPa or less, more preferably 0.1 kPa or more and 5 kPa or less, further preferably 0.12 kPa or more and 3 kPa or less. The vapor pressure of the rinsing liquid is 0.05 kPa or more and 5 kPa or less, thereby resulting in a more enhancement in temperature uniformity in a wafer surface, and also more suppression of swelling due to penetration of the rinsing liquid and a more improvement in dimension uniformity in a wafer surface.

The rinsing liquid, to which a proper amount of a surfactant is added, can also be used.

In the rinsing step, the wafer subjected to development is subjected to a washing treatment with the rinsing liquid including an organic solvent. The washing treatment method is not particularly limited, and, for example, a method (rotation coating method) including continuously discharging the rinsing liquid onto the substrate rotating at a certain speed, a method (dipping method) including dipping the substrate in a tank filled with the rinsing liquid, or a method (spraying method) including spraying the rinsing liquid on the substrate surface can be applied, and in particular, preferably, the washing treatment is performed according to the rotation coating method and thereafter the substrate after the washing is rotated at a number of rotations of 2000 rpm to 4000 rpm to thereby remove the rinsing liquid from the substrate.

After a resist pattern is formed, a pattern wiring substrate is obtained by etching. The etching method can be performed by a known method such as dry etching using plasma gas or wet etching with, for example, an alkali solution, a cupric chloride solution or a ferric chloride solution.

After a resist pattern is formed, plating can also be performed. Examples of the plating method include, but not particularly limited, copper plating, solder plating, nickel plating, and gold plating.

The remaining resist pattern after etching can be peeled by an organic solvent. Examples of the organic solvent include, but not particularly limited, PGMEA (propylene glycol monomethyl ether acetate), PGME (propylene glycol monomethyl ether), and EL (ethyl lactate). Examples of the peeling method include, but not particularly limited, a dipping method and a spraying system. The wiring substrate where the resist pattern is formed may be a multi-layer wiring substrate, and may have a small-sized through-hole.

The wiring substrate in the present embodiment can also be formed by a method including forming the resist pattern, then depositing a metal in vacuum and thereafter dissolving the resist pattern by a solution, namely, a liftoff method.

[Acid Generating Agent]

An acid generating agent according to the present embodiment includes the compound according to the present embodiment. The compound has an ion moiety having a specified structure at a terminal, and thus exhibits high sensitivity and provides high resolution and high flatness when used in an acid generating agent in a resist material or the like. The rate of diffusion of a molecule of the compound according to the present embodiment is appropriate, and thus high resolution is exhibited with high sensitivity being kept. The compound has an appropriate molecular weight, thus is hardly volatilized and allows for relatively low film reduction in curing, and thus allows high flatness to be exhibited. The acid generating agent according to the present embodiment generates an acid due to the action of heat or radiation. Examples of the radiation include g-ray, i-ray, KrF excimer laser, ArF excimer laser, extreme ultraviolet light (EUV), or electron beam. The acid generating agent according to the present embodiment may include other acid generating agent other than the compound according to the present embodiment.

[Second Composition]

A second composition according to the present embodiment includes the acid generating agent according to the present embodiment. The second composition according to the present embodiment can be, for example, a composition for underlayer film formation for lithography or a composition for optical article formation, but not limited thereto.

(Composition for Underlayer Film Formation for Lithography, Underlayer Film for Lithography, and Method for Forming Pattern)

### First Embodiment

<Composition for Underlayer Film Formation for Lithography>

A composition for underlayer film formation for lithography according to a first embodiment of the present invention is a composition for underlayer film formation for lithography, including an acid generating agent according to the present embodiment and a silicon-containing compound (for example, a hydrolyzable organosilane, a hydrolysate thereof or a hydrolysis condensate thereof). The composition for underlayer film formation for lithography of the present embodiment can form an underlayer film for lithography, such as a resist underlayer film, and is high in heat resistance and also high in solvent solubility. Thus, rectangularity of a pattern is excellent. The reduction of film defects (thin film formation) can be made, and high close contact, favorable storage stability, high sensitivity, long-term light resistance and a favorable resist pattern shape can be imparted. The composition for underlayer film formation for lithography of the present embodiment can form an underlayer film for lithography, which provides high flatness.

The composition for underlayer film formation for lithography of the present embodiment can be suitably used in, for example, a method with a multi-layer resist where a resist underlayer film is further provided between an upper layer resist (a photoresist or the like) and a hard mask or an organic underlayer film. In such a multi-layer resist method, for example, a resist underlayer film is formed on an organic underlayer film or a hard mask interposed on the substrate, by a coating method or the like, and an upper layer resist (for example, a photoresist, an electron beam resist, or an EUV resist) is formed on the resist underlayer film. A resist pattern is formed by exposure and development, the resist underlayer film is dry etched by use of the resist pattern, to thereby transfer the pattern, and the organic underlayer film is etched to thereby transfer the pattern and process the substrate by the organic underlayer film.

In other words, the underlayer film for lithography (resist underlayer film) formed by use of the composition for underlayer film formation for lithography of the present embodiment not only hardly causes intermixing with the upper layer resist, but also has heat resistance and is higher in etching rate against, for example, a halogen-based (fluorine-based) etching gas, than the upper layer resist patterned, for use as a mask, and thus can provide a rectangular and favorable pattern. Furthermore, the underlayer film for lithography (resist underlayer film) formed by use of the composition for underlayer film formation for lithography of the present embodiment is high in resistance against an oxygen-based etching gas, and thus can serve as a favorable mask in patterning of a layer provided on a base material, such as a hard mask. The composition for underlayer film formation for lithography of the present embodiment can also be used in a mode where a plurality of such resist underlayer films are laminated. In this case, such resist underlayer films formed by use of the composition for underlayer film formation for lithography of the present embodiment are not particularly limited in terms of the location thereof (the orders of such films laminated), and may be located immediately below the upper layer resist, may be layers located closest to the substrate, or may be in a mode where sandwiching between such resist underlayer films is made.

When a fine pattern is formed, a resist film thickness tends to be thinner in order to prevent pattern collapse. A resist film is thinner, and thus pattern transfer can be made only when dry etching for transferring a pattern to a film present as an underlayer exhibits a higher etching rate than the etching rate of a film as an upper layer. In the present embodiment, the organic underlayer film can be interposed on the substrate and can be covered with the resist underlayer film (containing a silicon-based compound) of the present embodiment, and the resultant can be further covered with a resist film (organic resist film). An organic component film and an inorganic component film are considerably different in dry etching rate due to selection of an etching gas, and the organic component film is increased in dry etching rate by an oxygen-based gas and the inorganic component film is increased in dry etching rate by a halogen-containing gas.

For example, a resist underlayer film where pattern transfer is made can be used for dry etching of an organic underlayer film as an underlayer by an oxygen-based gas to thereby perform pattern transfer to the organic underlayer film, and the organic underlayer film where pattern transfer is made can be used for processing of the substrate by use of a halogen-containing gas. The underlayer film for lithography (resist underlayer film) formed by use of the composition for underlayer film formation for lithography of the present embodiment is also favorable in close contact, and thus can also inhibit a pattern transferred, from being collapsed.

The resist underlayer film with the composition for underlayer film formation for lithography of the present embodiment includes the acid generating agent according to the present embodiment, excellent in absorption ability of active light, and a silicon-containing compound (for example, a hydrolyzable organosilane, a hydrolysate thereof or a hydrolysis condensate thereof), thereby resulting in an enhancement in sensitivity of the upper layer resist, causing no intermixing with the upper layer resist, and allowing the shape of a pattern on the film forming the resist underlayer film after exposure and development to be rectangular. Thus, substrate processing by a fine pattern is made possible.

The resist underlayer film with the composition for underlayer film formation for lithography of the present embodiment has high heat resistance and thus can be used even in high-temperature baking conditions. Furthermore, the molecular weight is relatively low and the viscosity is low, thus even a substrate having difference in level (in particular, a fine space and/or a hole pattern) can be filled uniformly in its every corner, resulting in a tendency to relatively advantageously increase flattening properties and embedding properties.

The composition for underlayer film formation for lithography can further include a solvent, an acid, an acid crosslinking agent, and the like, in addition to the acid generating agent according to the present embodiment and the silicon-containing compound. Furthermore, optional components such as an organic polymer compound and a surfactant, and other components such as water, an alcohol, and a curing catalyst can be included.

—Solvent—

A known solvent can be appropriately used as the solvent for use in the present embodiment as long as it dissolves at least the acid generating agent according to the present embodiment. Examples include any solvent which can be included in any composition for underlayer film formation for lithography, disclosed in International Publication No. WO2017/188450.

The content of the solvent is not particularly limited, and is preferably 100 to 10,000 parts by mass, more preferably 200 to 8,000 parts by mass, further preferably 200 to 5,000 parts by mass based on 100 parts by mass of the total solid content of the composition for underlayer film formation for lithography, from the viewpoints of solubility and film formation.

—Acid—

The composition for underlayer film formation for lithography can include an acid from the viewpoint of promotion of curability. Examples of the acid include hydrofluoric acid, hydrochloric acid, hydrobromic acid, sulfuric acid, nitric acid, perchloric acid, phosphoric acid, methanesulfonic acid, benzenesulfonic acid, and toluenesulfone.

The content of the acid is not particularly limited, and is preferably 0.001 to 20 parts by mass, more preferably 0.005 to 10 parts by mass, further preferably 0.01 to 5 parts by mass based on 100 parts by mass of the total solid content of the composition for underlayer film formation for lithography, from the viewpoints of solubility and shape stability of a coating film.

—Acid Crosslinking Agent—

In a case where the composition for underlayer film formation for lithography is used as a negative resist material or is used as an additive for an increase in strength of a pattern even in the case of a positive resist material, the underlayer film forming composition can include at least one acid crosslinking agent. Examples of the acid crosslinking agent can include a compound having at least one group (hereinafter, referred to as “crosslinkable group”) which can form crosslinking in the presence of an acid. Examples can include any acid crosslinking agent which can be included in any composition for underlayer film formation for lithography disclosed in International Publication No. WO2017/188450. Specific examples of the acid crosslinking agent can also include any agent described in International Publication No. WO2013/024779.

The content of the acid crosslinking agent is not particularly limited, and is preferably 0.01 to 30 parts by mass, more preferably 0.05 to 20 parts by mass, further preferably 0.1 to 10 parts by mass based on 100 parts by mass of the total solid content of the composition for underlayer film formation for lithography, from the viewpoints of solubility and shape stability of a coating film.

—Silicon-Containing Compound—

The composition for underlayer film formation for lithography includes not only the acid generating agent according to the present embodiment, but also a silicon-containing compound. The silicon-containing compound may be an organic silicon-containing compound or an inorganic silicon-containing compound, and is preferably an organic silicon-containing compound. Examples of the inorganic silicon-containing compound include polysilazane compounds made of silicon oxide, silicon nitride, and silicon oxide nitride, which can form a film according to a coating system at a low temperature. Examples of the organic silicon-containing compound include a polysilsesquioxane-based compound, a hydrolyzable organosilane, and a hydrolysate thereof or a hydrolysis condensate thereof. A specific material of the polysilsesquioxane-based compound, here used, can be, but are not limited to, for example, any material described in Japanese Patent Laid-Open No. 2007-226170 and Japanese Patent Laid-Open No. 2007-226204. The hydrolyzable organosilane, the hydrolysate thereof, or the hydrolysis condensate thereof can include at least one hydrolyzable organosilane selected from the group consisting of a hydrolyzable organosilane of the following formula (D1) and the following formula (D2), a hydrolysate thereof or a hydrolysis condensate thereof (hereinafter, these may be simply referred to as “at least one organosilicon compound selected from the group consisting of formula (D1) and formula (D2)”). The composition for underlayer film formation for lithography, when includes at least one organosilicon compound selected from the group consisting of formula (D1) and formula (D2), allows control of a Si—O bond by adjustment of curing conditions to be easy, also has the advantage of cost, and is suitable for introduction of an organic component. Thus, a layer formed by use of the composition for underlayer film formation for lithography, including at least one organosilicon compound selected from the group consisting of formula (D1) and formula (D2), is useful as an intermediate resist layer (a layer between an upper layer resist layer and an organic underlayer film provided on a base material).

(R³)ₐSi(R⁴)₄₋ₐ Formula (D1):

In formula (D1), R³ represents an “organic group” having an alkyl group, an aryl group, an aralkyl group, an alkyl halide group, an aryl halide group, an aralkyl halide group, an alkenyl group, an epoxy group, an acryloyl group, a methacryloyl group, a mercapto group, an alkoxyaryl group, an acyloxyaryl group, an isocyanurate group, a hydroxy group, a cyclic amino group, or a cyano group; or a combination thereof, and is bound to a silicon atom by a Si—C bond, R⁴ represents an alkoxy group, an acyloxy group or a halogen group, and a represents an integer of 0 to 3.

[(R⁵)_{c}Si(R⁶)_{4-c}]₂Y_{b} Formula (D2):

In formula (D2), R⁵ represents an alkyl group, R⁶ represents an alkoxy group, an acyloxy group or a halogen group, Y represents an alkylene group or an arylene group, b represents an integer of 0 or 1, and c represents an integer of 0 or 1.

The acid generating agent according to the present embodiment and the silicon-containing compound (for example, at least one organosilicon compound selected from the group consisting of formula (D1) and formula (D2)) in the composition for underlayer film formation for lithography can be used at proportions so that the molar ratio ranges from 0.1:99.9 to 50:50. For example, a molar ratio ranging from 1:99 to 30:70 can be adopted in order to obtain a favorable resist shape. At least one organosilicon compound selected from the group consisting of formula (D1) and formula (D2) is preferably used as the hydrolysis condensate (polyorganosiloxane polymer).

R³ in the hydrolyzable organosilane represented by formula (D1) represents an “organic group” having an alkyl group, an aryl group, an aralkyl group, an alkyl halide group, an aryl halide group, an aralkyl halide group, an alkenyl group, an epoxy group, an acryloyl group, a methacryloyl group, a mercapto group, an alkoxyaryl group, an acyloxyaryl group, an isocyanurate group, a hydroxy group, a cyclic amino group, or a cyano group, or a combination thereof, and is bound to a silicon atom by a Si—C bond, R⁴ represents an alkoxy group, an acyloxy group, or a halogen group, and a represents an integer of 0 to 3.

R⁵ in the hydrolyzable organosilane represented by formula (D2) represents an alkyl group, R⁶ represents an alkoxy group, an acyloxy group, or a halogen group, Y represents an alkylene group or an arylene group, b represents an integer of 0 or 1, and c represents an integer of 0 or 1.

Examples of the hydrolyzable organosilanes represented by formula (D1) and formula (D2) include any hydrolyzable organosilane which can be included in any composition for underlayer film formation for lithography disclosed in International Publication No. WO2017/188450.

In the present embodiment, the acid generating agent according to the present embodiment, and the hydrolyzable organosilane or the like may be in the form of a mixed product without any reaction, to thereby form a film, or the acid generating agent according to the present embodiment in the composition for underlayer film formation for lithography, and the above hydrolyzable organosilane or the like may be subjected to hydrolytic condensation with as an acid catalyst, at least one compound selected from an inorganic acid, an aliphatic sulfonic acid and an aromatic sulfonic acid.

Examples of the acid catalyst here used can include hydrofluoric acid, hydrochloric acid, hydrobromic acid, sulfuric acid, nitric acid, perchloric acid, phosphoric acid, methanesulfonic acid, benzenesulfonic acid, and toluenesulfonic acid. The amount of the catalyst used is preferably 10⁻⁶ to 10 mol, more preferably 10⁻⁵ to 5 mol, further preferably 10⁻⁴ to 1 mol per mol of a monomer (the total amount of the acid generating agent according to the present embodiment and the hydrolyzable organosilane or the like).

The amount of water added in hydrolytic condensation of the monomer is preferably 0.01 to 100 mol, more preferably 0.05 to 50 mol, further preferably 0.1 to 30 mol per mol of a hydrolyzable substituent bound to the monomer (the acid generating agent according to the present embodiment and the hydrolyzable organosilane or the like). An amount added of 100 mol or less is preferable from an economic viewpoint because an apparatus for use in such a reaction is not too large.

The operation method includes, for example, initiating a hydrolytic condensation reaction by addition of the monomer to an aqueous catalyst solution. An organic solvent may be added to the aqueous catalyst solution or the monomer may be diluted with an organic solvent, or both may be performed. The reaction temperature is preferably 0 to 100° C., more preferably 40 to 100° C. A method is preferable where the temperature in dropping of the monomer is kept at 5 to 80° C. and thereafter aging at 40 to 100° C. is made.

Examples of the organic solvent which can be added to the aqueous catalyst solution or which can dilute the monomer include any organic solvent disclosed in International Publication No. WO2017/188450.

The amount of the organic solvent used is preferably 0 to 1,000 ml, particularly preferably 0 to 500 ml per mol of the monomer (the total amount of the acid generating agent according to the present embodiment and the hydrolyzable organosilane or the like). An amount of the organic solvent used, of 1,000 ml or less, is preferable from an economic viewpoint because a reaction container is not too large.

Thereafter, a neutralization reaction of the catalyst is performed when needed, and an alcohol generated in the hydrolytic condensation reaction is removed under reduced pressure to thereby provide an aqueous reaction mixture solution. The amount of an alkaline substance here usable in neutralization is preferably 0.1 to 2 equivalents relative to the acid used in the catalyst. The alkaline substance may be any substance as long as alkalinity is exhibited in water.

Subsequently, a by-product, for example, an alcohol generated from a reaction mixture in the hydrolytic condensation reaction is preferably removed. The heating temperature of the reaction mixture is preferably 0 to 100° C., more preferably 10 to 90° C., further preferably 15 to 80° C., while depends on the types of the organic solvent added and the alcohol generated in the reaction. The degree of depressurization here is preferably an atmospheric pressure or less, more preferably 80 kPa or less as an absolute pressure, further preferably 50 kPa or less as an absolute pressure, while differs depending on the types of the organic solvent, the alcohol and the like to be removed, an exhaust apparatus, a condensing apparatus, and the heating temperature. While the amount of the alcohol removed is difficult to precisely find, it is desirable to remove about 80 mass % or more of the alcohol generated or the like.

Next, the acid catalyst used m the hydrolytic condensation may be removed from the reaction mixture. The method for removing the acid catalyst can be, for example, a method including mixing water and the reaction mixture, and extracting a product with an organic solvent. The organic solvent here used is preferably one which can dissolve the product and which allows for separation into two layers when mixed with water. Examples include any organic solvent disclosed in International Publication No. WO2017/188450.

When the acid catalyst used in the hydrolytic condensation is removed from the reaction mixture, a mixture of a water-soluble organic solvent and a poorly water-soluble organic solvent can also be used. Examples include any mixture disclosed in International Publication No. WO2017/188450.

The mixing ratio of the water-soluble organic solvent and the poorly water-soluble organic solvent is appropriately selected, and the proportion of the water-soluble organic solvent based on 100 parts by mass of the poorly water-soluble organic solvent is preferably 0.1 to 1,000 parts by mass, more preferably 1 to 500 parts by mass, further preferably 2 to 100 parts by mass.

Even in both the case of a product in which the acid catalyst remains and the case of a product from which the acid catalyst is removed, a solution of such a product can be obtained by adding a final solvent and performing solvent exchange under reduced pressure. The temperature in the solvent exchange is preferably 0 to 100° C., more preferably 10 to 90° C., further preferably 15 to 80° C., while depends on the types of the reaction solvent and the extraction solvent to be removed. The degree of depressurization here is preferably an atmospheric pressure or less, more preferably 80 kPa or less as an absolute pressure, further preferably 50 kPa or less as an absolute pressure, while differs depending on the type of the extraction solvent to be removed, an exhaust apparatus, a condensing apparatus, and the heating temperature.

—Other Optional Component—

The composition for underlayer film formation for lithography can, if necessary, include an organic polymer compound, a crosslinking agent, a surfactant or the like, in addition to the above components.

The organic polymer compound can be used to thereby adjust, for example, the dry etching rate (the amount of the film thickness reduced per unit time), the attenuation coefficient and the refractive index of a resist underlayer film formed from the composition for underlayer film formation for lithography. The organic polymer compound is not particularly limited, and various organic polymers can be used. A polycondensation polymer, an addition polymerization polymer, or the like can be used. For example, any organic polymer compound disclosed in International Publication No. WO2017/188450 can be used.

The crosslinking agent can be used to thereby adjust, for example, the dry etching rate (the amount of the film thickness reduced per unit time) of a resist underlayer film formed from the composition for underlayer film formation for lithography. The crosslinking agent is not particularly limited, and various crosslinking agents can be used. Specific examples of the crosslinking agent usable in the present embodiment include a melamine compound, a guanamine compound, a glycoluryl compound, a urea compound, an epoxy compound, a thioepoxy compound, an isocyanate compound, an azide compound, and a compound containing a double bond such as an alkenyl ether group, the compounds each having at least one group selected from a methylol group, an alkoxymethyl group and an acyloxymethyl group, as a substituent (crosslinkable group), but not particularly limited thereto. Examples include any crosslinking agent disclosed in International Publication No. WO2017/188450.

The content of the crosslinking agent in the composition for underlayer film formation for lithography is not particularly limited, and is preferably 1 to 10 parts by mass, more preferably 1 to 5 parts by mass based on 100 parts by mass of the acid generating agent according to the present embodiment. The content is in the above preferable range to result in a tendency to suppress the occurrence of a mixing phenomenon with a resist layer and also tendencies to enhance an antireflective effect and enhance film formability after crosslinking.

The surfactant is effective for inhibiting surface defects or the like from occurring in coating of the substrate with the composition for underlayer film formation for lithography. Examples of the surfactant included in the composition for underlayer film formation for lithography include any surfactant disclosed in International Publication No. WO2017/188450. In a case where the surfactant is used, the proportion thereof can be, for example, 0 parts by mass to 5 parts by mass based on 100 parts by mass of the acid generating agent according to the present embodiment.

<Underlayer Film for Lithography and Method for Forming Pattern>

An underlayer film for lithography according to the first embodiment of the present invention can be formed by use of the composition for underlayer film formation for lithography according to the first embodiment of the present invention. The underlayer film for lithography of the present embodiment can be suitably used as an underlayer (resist underlayer film) of a photoresist (upper layer) for use in a multi-layer resist method.

In the present embodiment, a pattern can be formed by, for example, forming a resist underlayer film by use of the composition for underlayer film formation for lithography, forming at least one photoresist layer on the resist underlayer film, and thereafter irradiating a predetermined region of the photoresist layer with radiation to thereby perform development.

One aspect of the method for forming a pattern according to the first embodiment of the present invention by use of the composition for underlayer film formation for lithography according to the first embodiment of the present invention, produced as described above, can provide, for example, a method for forming a pattern, including forming an organic underlayer film on a substrate, with a coating type organic underlayer film material, forming a resist underlayer film on the organic underlayer film, by use of the composition for underlayer film formation for lithography of the first embodiment of the present invention, forming an upper layer resist film on the resist underlayer film, by use of an upper layer resist film composition, forming an upper layer resist pattern on the upper layer resist film, transferring a pattern to the resist underlayer film by etching with the upper layer resist pattern as a mask, transferring a pattern to the organic underlayer film by etching with the resist underlayer film to which a pattern is transferred, as a mask, and further transferring a pattern to the substrate (object to be processed) by etching with the organic underlayer film to which a pattern is transferred, as a mask.

Another aspect of the method for forming a pattern according to the first embodiment of the present invention can provide, for example, a method for forming a pattern, including forming an organic hard mask containing carbon as a main component, on a substrate, by a CVD method, forming a resist underlayer film on the organic hard mask, by use of the composition for underlayer film formation for lithography of the first embodiment of the present invention, forming an upper layer resist film on the resist underlayer film, by use of an upper layer resist film composition, forming an upper layer resist pattern on the upper layer resist film, transferring a pattern to the resist underlayer film by etching with the upper layer resist pattern as a mask, transferring a pattern to the organic hard mask by etching with the resist underlayer film to which a pattern is transferred, as a mask, and further transferring a pattern to the base material (object to be processed) by etching with the organic hard mask to which a pattern is transferred, as a mask.

The base material here used can be, for example, a semiconductor substrate. A silicon substrate can be commonly used as the semiconductor substrate, and any substrate of Si, amorphous silicon (α-Si), p-Si, SiO₂, SiN, SiON, W. TiN, Al, or the like, different in material from a layer to be processed, can be used without any particularly limitation.

A metal constituting the base material (object to be processed; including the semiconductor substrate) here used can be any of silicon, titanium, tungsten, hafnium, zirconium, chromium, germanium, copper, aluminum, indium, gallium, arsenicum, palladium, iron, tantalum, iridium, or molybdenum, or an alloy thereof.

A semiconductor substrate on which any of a metal film, a metal carbide film, a metal oxide film, a metal nitride film, a metal oxycarbide film, or a metal oxynitride film is formed as a layer to be processed (portion to be processed) can be used. Such a metal-containing layer to be processed, here used, is, for example, any of Si, SiO₂, SiN, SiON, SiOC, p-Si, α-Si, TiN, WSi, BPSG, SOG, Cr, CrO, CrON, MoSi, W, W—Si, Al, Cu, Al—Si, and the like, and various low-dielectric films and etching stopper films thereof, and such a film can be formed so as to usually have a thickness of 50 to 10,000 nm, in particular, 100 to 5,000 nm.

An organic underlayer film or an organic hard mask can be formed on the substrate in the method for forming a pattern of the present embodiment. In particular, the organic underlayer film can be formed from a coating type organic underlayer film material, by a rotation coating method or the like, and the organic hard mask can be formed from a material of an organic hard mask containing carbon as a main component, by a CVD method. The organic underlayer film and the organic hard mask are not particularly limited in terms of the types thereof, and are each preferably one which exhibits a sufficient antireflective film function in a case where the upper layer resist film is exposed to thereby form a pattern. The organic underlayer film or the organic hard mask can be formed to thereby transfer the pattern formed based on the upper layer resist film, onto the base material (object to be processed), without the occurrence of any difference in size conversion. Herein, the hard mask “containing carbon as a main component” means a hard mask in which 50 mass % or more of the solid content is constituted from a carbon-based material of amorphous hydrogenated carbon or the like called also “amorphous carbon” and represented by a-C:H. While an a-C:H film can be deposited by various techniques, plasma-enhanced chemical vapor deposition (PECVD) is widely used to achieve cost efficiency and to enable film quality to be adjusted. Examples of the hard mask can be seen in those described in, for example, Japanese Translation of PCT International Application Publication No. 2013-526783.

The resist underlayer film with the composition for resist underlayer film formation of the present embodiment, for use in the method for forming a pattern of the present embodiment, can be produced on an object to be processed, on which the organic underlayer film or the like is provided from the composition for underlayer film formation for lithography, by a spin coating method or the like. In a case where the resist underlayer film is formed by a spin coating method, baking is desirably made after spin coating in order to evaporate a solvent and promote a crosslinking reaction for the purpose of prevention of mixing with the upper layer resist film. The baking temperature is preferably in the range from 50 to 500° C. The baking temperature is here particularly preferably 400° C. or less in order to decrease thermal damage to the device, while depends on the structure of a device to be produced. The baking time is preferably in the range from 10 seconds to 300 seconds.

The method for forming a pattern on the upper layer resist film, adopted in the method for forming a pattern of the present embodiment, can be suitably any method of a lithography method using light at a wavelength of 300 nm or less or EUV light; an electron beam direct drawing method, and an inductive self-organization method. Such a method can be used to thereby form a fine pattern on the upper layer resist film.

The upper layer resist film composition can be appropriately selected depending on the method for forming a pattern on the upper layer resist film. For example, in the case of lithography with light of 300 nm or less, or EUV light, a chemical amplification type photoresist film material can be used as the upper layer resist film composition. Examples of such a photoresist film material can include a material for forming a positive pattern by formation of a photoresist film and exposure thereof, and then dissolution of an exposed region with an alkaline developing solution, and a material for forming a negative pattern by such formation and exposure and then dissolution of an unexposed region with a developer including an organic solvent.

A resist underlayer film formed from the composition for underlayer film formation for lithography of the present embodiment may absorb the light, while depends on the wavelength of light for use in a lithography process. In this case, the film can function as an antireflective film having the effect of preventing light reflected from the substrate.

An EUV resist underlayer film not only functions as a hard mask, but also can be used for the following purpose. The composition for underlayer film formation for lithography according to the present embodiment can be used as an underlayer antireflective film of EUV resist, which does not intermix with the EUV resist and can prevent reflection of undesirable exposure light in EUV exposure (wavelength 13.5 nm), for example, the above UV and DUV (ArF light, KrF light), from the substrate or interface. Such an underlayer of EUV resist can efficiently prevent reflection. The composition for underlayer film formation is excellent in absorption ability of EUV, and thus can exhibit sensitization of an upper layer resist composition, and contributes to an enhancement in sensitivity. In the case of use in such an EUV resist underlayer film, a process as in that of an underlayer film for photoresists can be performed.

### Second Embodiment

<Composition for Underlayer Film Formation for Lithography>

A composition for underlayer film formation for lithography according to a second embodiment of the present invention is a composition for underlayer film formation for lithography, including an acid generating agent according to the present embodiment. The composition for underlayer film formation for lithography of the present embodiment can allow for the reduction of film defects (thin film formation), is favorable in storage stability, is high in sensitivity and has long-term light resistance, and can provide a favorable resist pattern shape. The composition for underlayer film formation for lithography of the present embodiment can include no silicon-containing compound.

The composition for underlayer film formation for lithography of the present embodiment can be applied to a wet process, and a composition for underlayer film formation for lithography can be realized which is useful for formation of a photoresist underlayer film excellent in heat resistance, close contact, and level embedding properties, in particular, flatness. A compound capable of allowing for a relatively high crosslinking density, having high solvent solubility, and having a specified structure is used in the composition for underlayer film formation for lithography, and thus an underlayer film not only suppressed in degradation during baking, but also excellent in etching resistance to fluorine gas-based plasma etching or the like can be formed. Furthermore, close contact with a resist layer is also excellent, and thus an excellent resist pattern can be formed. The composition for underlayer film formation for lithography of the present embodiment particularly has excellent heat resistance and level embedding properties, and provides excellent flatness, and thus, for example, can be used as a composition for forming a resist underlayer film provided as the undermost layer among a plurality of resist layers. Herein, the resist underlayer film formed by use of the composition for underlayer film formation for lithography of the present embodiment may further include other resist underlayer between the film and the substrate.

The composition for underlayer film formation for lithography according to the present embodiment can further include a solvent, an acid crosslinking agent, and the like, in addition to the acid generating agent according to the present embodiment. A basic compound, and furthermore water, an alcohol, and a curing catalyst, as optional components can be further included. The content of the acid generating agent according to the present embodiment in the composition for underlayer film formation for lithography is preferably 0.001 to 49 mass %, more preferably 1 to 40 mass %, particularly preferably 3 to 30 mass %, from the viewpoints of coatability and quality stability.

—Solvent—

A known solvent can be appropriately used as the solvent for use in the present embodiment as long as it dissolves at least the acid generating agent according to the present embodiment. Examples include any solvent disclosed in International Publication No. WO2017/188451.

The content of the solvent is not particularly limited, and is preferably 100 to 10,000 parts by mass, more preferably 200 to 5,000 parts by mass, further preferably 200 to 1,000 parts by mass based on 100 parts by mass of the total solid content of the composition for underlayer film formation for lithography from the viewpoints of solubility and film formation.

—Acid Crosslinking Agent—

As described above, the composition for underlayer film formation for lithography of the present embodiment may, if necessary, include an acid crosslinking agent from the viewpoint of, for example, suppression of intermixing. Examples of the acid crosslinking agent usable in the present embodiment include a melamine compound, an epoxy compound, a guanamine compound, a glycoluryl compound, a urea compound, a thioepoxy compound, an isocyanate compound, an azide compound, and a compound containing a double bond such as an alkenyl ether group, the compounds each having at least one group selected from a methylol group, an alkoxymethyl group and an acyloxymethyl group, as a substituent (crosslinkable group), but not particularly limited thereto. These acid crosslinking agents can be used singly or in combinations of two or more kinds thereof. These may also be each used as an additive. A compound containing a hydroxy group can also be used as the crosslinking agent. Specific examples of the acid crosslinking agent include any acid crosslinking agent described in International Publication No. WO 2013/024779.

The content of the acid crosslinking agent in the composition for underlayer film formation for lithography of the present embodiment is not particularly limited, and is preferably 5 to 50 parts by mass, more preferably 10 to 40 parts by mass based on 100 parts by mass of the total solid content of the composition for underlayer film formation for lithography. The content is in the above preferable range to result in a tendency to suppress the occurrence of a mixing phenomenon with a resist layer and also tendencies to enhance an antireflective effect and enhance film formability after crosslinking.

—Basic Compound—

The composition for underlayer film formation for lithography of the present embodiment may further include a basic compound from the viewpoint of, for example, an enhancement in storage stability.

The basic compound serves as a quencher of an acid, which prevents an acid generated in a trace amount from the acid generating agent from progressing a crosslinking reaction. Examples of the basic compound include any primary, secondary or tertiary aliphatic amine, any mixed amine, any aromatic amine, any heterocyclic amine, any nitrogen-containing compound having a carboxy group, any nitrogen-containing compound having a sulfonyl group, any nitrogen-containing compound having a hydroxy group, any nitrogen-containing compound having a hydroxyphenyl group, any alcoholic nitrogen-containing compound, any amide derivative, or any imide derivative, but not particularly limited thereto. Specific examples of the basic compound include any basic compound described in International Publication No. WO 2013/024779.

The content of the basic compound in the composition for underlayer film formation for lithography of the present embodiment is not particularly limited, and is preferably 0.001 to 2 parts by mass, more preferably 0.01 to 1 parts by mass based on 100 parts by mass of the total solid content of the composition for underlayer film formation for lithography. The content is in the above preferable range to result in a tendency to enhance storage stability without excessive loss of the crosslinking reaction.

The composition for underlayer film formation for lithography of the present embodiment may include other resin or compound for the purposes of imparting thermosetting properties and controlling absorbance. Examples of such other resin or compound include a naphthol resin, a xylene resin, a naphthol-modified resin, a phenol-modified resin of a naphthalene resin, polyhydroxystyrene, a dicyclopentadiene resin, (meth)acrylate, dimethacrylate, trimethacrylate, tetramethacrylate, a resin containing a naphthalene ring, such as vinylnaphthalene and polyacenaphthylene, a resin containing a biphenyl ring, such as phenanthraquinone and fluorene, a resin containing a heterocyclic ring having a hetero atom, such as thiophene and indene, and a resin containing no aromatic ring; a rosin-based resin, a resin or a compound containing an alicyclic structure, such as cyclodextrin, adamantane (poly)ol, tricyclodecane (poly)ol, and derivatives thereof, but not particularly limited thereto. The composition for underlayer film formation for lithography of the present embodiment may further include a known additive. Examples of such a known additive include, but are not limited to, an ultraviolet absorbent, a surfactant, a colorant, and a nonionic surfactant.

<Resist Underlayer Film for Lithography and Method for Forming Pattern>

A resist underlayer film for lithography according to the second embodiment of the present invention is formed by use of the composition for underlayer film formation for lithography according to the second embodiment of the present invention. A pattern formed in the present embodiment can be used as, for example, a resist pattern or a circuit pattern.

A method for forming a pattern according to the second embodiment of the present invention includes a step (step A-1) of forming a resist underlayer film on a substrate, by use of the composition for underlayer film formation for lithography of the second embodiment of the present invention, a step (step A-2) of forming at least one photoresist layer on the resist underlayer film, and a step (step A-3) of, after formation of the at least one photoresist layer in step A-2, irradiating a predetermined region of the photoresist layer with radiation to thereby perform development. The “photoresist layer” means a layer provided in the outermost layer of the resist layer, namely, the outermost surface of the resist layer (opposite to the substrate).

Other method for forming a pattern of the second embodiment of the present invention includes a step (step B-1) of forming a resist underlayer film on a substrate, by use of the composition for underlayer film formation for lithography of the second embodiment of the present invention, a step (step B-2) of forming a resist intermediate layer film on the underlayer film, by use of a resist intermediate layer film material (for example, silicon-containing resist layer), a step (step B-3) of forming at least one photoresist layer on the resist intermediate layer film, a step (step B4) of, after formation of the at least one photoresist layer in step B-3, irradiating a predetermined region of the photoresist layer with radiation to thereby perform development and thus form a resist pattern, and a step (step B-5) of, after formation of the resist pattern in step B-4, etching the resist intermediate layer film with the resist pattern as a mask, etching the underlayer film with the resulting intermediate layer film pattern, as an etching mask, and etching the substrate with the resulting underlayer film pattern as an etching mask to thereby form a pattern on the substrate.

The method for forming the resist underlayer film for lithography of the present embodiment is not particularly limited as long as the resist underlayer film is formed from the composition for underlayer film formation for lithography of the present embodiment, and a known procedure can be applied. For example, the resist underlayer film can be formed by applying the composition for underlayer film formation for lithography of the present embodiment to a substrate by a known coating method or printing method such as spin coating or screen printing, and thereafter removing an organic solvent by volatilization or the like.

The resist underlayer film, when formed, is preferably subjected to baking treatment in order to not only suppress the occurrence of a mixing phenomenon with an upper layer resist (for example, a photoresist layer or a resist intermediate layer film), but also promote a crosslinking reaction. In this case, the baking temperature is not particularly limited, and is preferably in the range from 80 to 450° C., more preferably 200 to 400° C. The baking time is also not particularly limited, and is preferably in the range from 10 seconds to 300 seconds. The thickness of the resist underlayer film can be appropriately selected depending on requirement performance, is not particularly limited, and is usually preferably about 30 to 20,000 nm, more preferably 50 to 15,000 nm.

After the resist underlayer film is produced on the substrate, a resist intermediate layer film can be provided between the photoresist layer and the resist underlayer film. For example, in the case of a two-layer process, a silicon-containing resist layer, a single-layer resist including usual hydrocarbon, or the like can be provided as a resist intermediate layer film on the resist underlayer film. For example, in the case of a three-layer process, preferably, a silicon-containing intermediate layer is produced between the resist intermediate layer film and the photoresist layer, and furthermore a single-layer resist layer containing no silicon is produced thereon. The photoresist materials for use in formation of the photoresist layer, the resist intermediate layer film, and the resist layer provided therebetween can be known photoresist materials.

For example, the silicon-containing resist material for use in the two-layer process is preferably a positive photoresist material using a silicon atom-containing polymer such as a polysilsesquioxane derivative or a vinylsilane derivative, as a base polymer, and further including an organic solvent and, if necessary, a basic compound or the like, from the viewpoint of oxygen gas etching resistance. The silicon atom-containing polymer here used can be a known polymer for use in such a resist material.

For example, the silicon-containing intermediate layer for use in a three-layer process is preferably a polysilsesquioxane-based intermediate layer. The resist intermediate layer film is allowed to have the effect of an antireflective film, resulting in a tendency to enable reflection to be effectively suppressed. For example, if a material including many aromatic groups and having high substrate etching resistance is used in the resist underlayer film in a process for 193-nm exposure, a high k value and high substrate reflection tend to be caused, but the resist intermediate layer can suppress reflection to thereby allow substrate reflection to be 0.5% or less. Such an intermediate layer having an antireflective effect, here used for 193-nm exposure, is preferably, but are not limited to, polysilsesquioxane into which a phenyl group or a light absorption group having a silicon-silicon bond is introduced and which is to be crosslinked by an acid or heat.

A resist intermediate layer film formed by a Chemical Vapour Deposition (CVD) method can also be used. For example, a SiON film is known as an intermediate layer highly effective as an antireflective film produced by a CVD method, but not limited thereto. In general, formation of a resist intermediate layer film by a wet process such as a spin coating method or screen printing is simple and cost-effective as compared with by a CVD method. An upper layer resist in a three-layer process may be positive or negative, and the same resist as the single-layer resist usually used can be used.

A resist underlayer film of the present embodiment can also be used as an antireflective film for a usual single-layer resist or as an underlying material for suppression of pattern collapse. The resist underlayer film of the present embodiment is excellent in etching resistance for underlying processing, and thus can also be expected to function as a hard mask for underlying processing.

In a case where a resist layer is formed by the above known photoresist material, a wet process such as a spin coating method or screen printing is preferably used as in the case of formation of the resist underlayer film. After coating with the resist material according to a spin coating method or the like, pre-baking is usually performed, and the pre-baking is preferably performed at a baking temperature ranging from 80 to 180° C. for a baking time ranging from 10 seconds to 300 seconds. Thereafter, exposure can be performed and post-exposure baking (PEB) and development can be performed according to ordinary methods, to thereby provide a resist pattern. The thickness of each resist film is not particularly limited, and is generally preferably 30 nm to 500 nm, more preferably 50 nm to 400 nm.

The exposure light may be appropriately selected and used depending on the photoresist material used. Examples can commonly include high energy line having a wavelength of 300 nm or less, specifically, excimer laser at 248 nm, 193 nm and 157 nm, soft X-ray at 3 to 20 nm, electron beam, and X-ray.

A resist pattern formed by the above method is suppressed in pattern collapse by the resist underlayer film of the present embodiment. Therefore, the resist underlayer film of the present embodiment can be used to thereby obtain a finer pattern and also reduce the amount of exposure necessary for obtaining such a resist pattern.

Next, the resulting resist pattern is used as a mask to perform etching. Gas etching is preferably used as etching of the resist underlayer film in a two-layer process. Gas etching is suitably etching using an oxygen gas. Not only an oxygen gas, but also an inert gas such as He or Ar, and/or CO, CO₂, NH₃, SO₂, N₂, NO₂, and/or H₂ gas(es) can also be added. Gas etching can also be performed without use of any oxygen gas by using only CO, CO₂, NH₃, N₂, NO₂, and/or H₂ gas(es). In particular, the latter gas(es) is/are preferably used for side wall protection for prevention of undercutting of a pattern side wall.

Gas etching is preferably used also as etching of the intermediate layer (the layer located between the photoresist layer and the resist underlayer film) in a three-layer process. Gas etching here applied can be the same as that described above with respect to a two-layer process. In particular, processing of the intermediate layer in a three-layer process is preferably performed using a fluorocarbon gas with a resist pattern as a mask. Thereafter, for example, oxygen gas etching can be performed with an intermediate layer pattern as a mask, as described above, thereby performing processing of the resist underlayer film.

In a case where an inorganic hard mask intermediate layer film is formed as the intermediate layer, a silicon oxide film, a silicon nitride film, or a silicon oxide nitride film (SiON film) is formed by a CVD method, an ALD method, or the like. The method for forming a nitride film can be, but are not limited to, for example, any method described in Japanese Patent Laid-Open No. 2002-334869 or WO2004/066377. While a photoresist film can be formed directly on such an intermediate layer film, a photoresist film may be formed on an organic antireflective film (BARC) formed on such an intermediate layer film by spin coating.

A polysilsesquioxane-based intermediate layer is also preferably used as the intermediate layer. A resist intermediate layer film can be allowed to have the effect of an antireflective film, to result in a tendency to effectively suppress reflection. A specific material of the polysilsesquioxane-based intermediate layer, here used, can be, but are not limited to, any material described in, for example, Japanese Patent Laid-Open No. 2007-226170 or Japanese Patent Laid-Open No. 2007-226204.

Etching of the substrate can also be performed by an ordinary method, and, for example, etching mainly with a fluorocarbon gas can be performed in a case where the substrate is made of SiO₂ or SiN, and etching mainly with a chlorine-based or bromine-based gas can be performed in a case where the substrate is made of p-Si, Al, or W. In a case where the substrate is etched with a fluorocarbon gas, the silicon-containing resist in a two-layer resist process and the silicon-containing intermediate layer in a three-layer process are peeled at the same time as processing of the substrate. In a case where the substrate is etched with a chlorine-based or bromine-based gas, the silicon-containing resist layer or the silicon-containing intermediate layer is separately peeled, generally, peeled by dry etching with a fluorocarbon gas after processing of the substrate.

The resist underlayer film of the present embodiment is excellent in etching resistance of the substrate. The substrate can be appropriately selected from known substrates and used, and is not particularly limited, and examples thereof include Si, α-Si, p-Si, SiO₂, SiN, SiON, W, TiN, and Al. The substrate may be a laminate having on a base material (support), a film to be processed (substrate to be processed). Examples of the film to be processed include various Low-k films of Si, SiO₂, SiON, SiN, p-Si, α-Si, W, W—Si, Al, Cu, Al—Si, and the like, and stopper films thereof, and any substrate whose material is different from the base material (support) is usually used. The thickness of the substrate of interest to be processed or the film to be processed is not particularly limited, and is usually preferably about 50 nm to 10,000 nm, more preferably 75 nm to 5,000 nm.

The resist underlayer film of the present embodiment is excellent in embedding flatness to a substrate having difference in level. A known evaluation method can be appropriately selected and used as the method for evaluating the embedding flatness, and the embedding flatness to a substrate having difference in level can be evaluated, without any particular limitation, by, for example, coating a silicon substrate having difference in level, with a solution of each compound having a predetermined concentration adjusted, by spin coating, performing drying and removal of a solvent at 110° C. for 90 seconds to thereby form an underlayer film so that a predetermined thickness is achieved, and thereafter measuring the difference (AT) in underlayer film thickness between a line and space region and an opening region having no pattern after baking at a temperature of about 240 to 300° C. for a predetermined time, with an ellipsometer.

(Composition for Optical Article Formation and Optical Article)

A composition for optical component formation according to the present embodiment is a composition for optical component formation, including the acid generating agent according to the present embodiment. The composition for optical component formation is usefully used for formation of an optical article. The composition for optical component formation of the present embodiment includes the acid generating agent according to the present embodiment, and thus the resulting optical article can be expected to have a high refractive index and high transparency, and furthermore storage stability, structure-forming ability (film-forming ability), and heat resistance.

The refractive index of the optical article is preferably 1.65 or more, more preferably 1.70 or more, further preferably 1.75 or more from the viewpoints of a reduction in size of an optical component and an enhancement in light collection rate. The transparency of the optical article is preferably 70% or more, more preferably 80/or more, further preferably 90% or more from the viewpoint of an enhancement in light collection rate.

The method for measuring the refractive index is not particularly limited and a known method is used. Examples include a spectroscopic ellipsometry method, a minimum deviation method, a critical angle method (Abbe's system or Pulfrich's system), a V-block method, a prism coupler method, and an immersion method (Becke's line method). The method for measuring the transparency is not particularly limited and a known method is used. Examples include a method with a spectrophotometer and a spectroscopic ellipsometry method.

A cured product according to the present embodiment, forming an optical article obtained by curing of the composition for optical component formation, can be a three-dimensionally crosslinked product, is suppressed in coloration by a heat treatment in a broad range from a low temperature to a high temperature, and can be expected to have a high refractive index and high transparency.

The composition for optical component formation of the present embodiment can further include a solvent, in addition to the acid generating agent according to the present embodiment. The solvent can be the same as the solvent for use in the composition for underlayer film formation for lithography of the present embodiment, described above.

The relationship between the amount of the solid component and the amount of the solvent in the composition for optical component formation of the present embodiment is not particularly limited, and is preferably a relationship between 1 to 80 mass % of the solid component and 20 to 99 mass % of the solvent, more preferably 1 to 50 mass % of the solid component and 50 to 99 mass % of the solvent, further preferably 2 to 40 mass % of the solid component and 60 to 98 mass % of the solvent, particularly preferably 2 to 10 mass % of the solid component and 90 to 98 mass % of the solvent, based on 100 mass % of the total of the solid component and the solvent. The composition for optical component formation of the present embodiment can also include no solvent.

The composition for optical component formation of the present embodiment may include at least one selected from the group consisting of an acid crosslinking agent (G), an acid diffusion controlling agent (E) and other component (F), as other solid component.

The content of the acid generating agent according to the present embodiment in the composition for optical component formation of the present embodiment is not particularly limited, and is preferably 0.001 to 49 mass %, more preferably 1 to 40 mass %, further preferably 3 to 30 mass %, particularly preferably 3 to 20 mass %, based on the total mass of the solid component (the sum of the acid generating agent according to the present embodiment, and the solid component(s) optionally used, for example, the acid crosslinking agent (G), the acid diffusion controlling agent (E) and other component (F), the same applies to the following).

—Acid Crosslinking Agent (G)—

In a case where the composition for optical component formation of the present embodiment is used as an additive for an increase in strength of a structure, it preferably includes at least one acid crosslinking agent (G). The acid crosslinking agent (G) is not particularly limited, and can be the same as the acid crosslinking agent (G) which can be included in the composition for underlayer film formation for lithography of the present embodiment, described above.

The content of the acid crosslinking agent (G) in the composition for optical component formation of the present embodiment is preferably 0.5 to 49 mass %, more preferably 0.5 to 40 mass %, further preferably 1 to 30 mass %, particularly preferably 2 to 20 mass %, based on the mass of the total solid component. The content ratio of the acid crosslinking agent (G) is preferably 0.5 mass % or more because the effect of suppressing solubility of the composition for optical component formation in an organic solvent can be enhanced, and on the other hand, the content ratio is preferably 49 mass % or less because deterioration in heat resistance of the composition for optical component formation can be suppressed.

The content of at least one compound selected from the acid crosslinking agent (G1), the acid crosslinking agent (G2), and the acid crosslinking agent (G3) in the acid crosslinking agent (G) is also not particularly limited, and can fall within various ranges depending on the type of the substrate for use in formation of the composition for optical component formation.

—Acid Diffusion Controlling Agent (E)—

The composition for optical component formation of the present embodiment may include an acid diffusion controlling agent (E) which has the effect of controlling diffusion of an acid generated from the acid generating agent, in the composition for optical component formation, to thereby inhibit an undesirable chemical reaction. The acid diffusion controlling agent (E) is used to result in an enhancement in preservation stability of the composition for optical component formation. Additionally, not only the resolution is further enhanced, but also a structure can be inhibited from being changed in line width due to the variation in post exposure delay after heating, and is extremely excellent in process stability. The acid diffusion controlling agent (E) is not particularly limited, and can be, for example, the same as the acid diffusion controlling agent (E) which can be included in the composition for underlayer film formation for lithography of the present embodiment.

The content of the acid diffusion controlling agent (E) is preferably 0.001 to 49 mass %, more preferably 0.01 to 10 mass %, further preferably 0.01 to 5 mass %, particularly preferably 0.01 to 3 mass %, based on the mass of the total solid component. When the content of the acid diffusion controlling agent (E) is in the above range, a reduction in resolution, and degradation of a pattern shape, dimensional faithfulness, and the like can be further suppressed. Furthermore, even if the post exposure delay after irradiation with radiation from irradiation with electron beam is increased, the shape of the upper layer portion of a pattern is not degraded. When the content of the acid diffusion controlling agent (E) is 10 mass % or less, sensitivity, developability in an unexposed region, and the like can be prevented from being deteriorated. The acid diffusion controlling agent is used to not only result in an enhancement in preservation stability of the composition for optical component formation and an enhancement in resolution, but also enable the change in line width of the composition for optical component formation, due to the variations in post exposure delay before irradiation with radiation and post exposure delay after irradiation with radiation, to be suppressed, and allow for extremely excellent process stability.

—Other Component (F)—

One or more of various additives such as a dissolution promoting agent, a dissolution controlling agent, a sensitizing agent, a surfactant, and an organic carboxylic acid or an oxo acid of phosphorus, or any derivative thereof can be, if necessary, added as other component (F) to the composition for optical component formation of the present embodiment as long as the objects of the present embodiment are not impaired. Such other component (F) can be the same as, for example, other component (F) described above which can be included in the composition for underlayer film formation for lithography of the present embodiment.

The total content of such other component (F) is preferably 0 to 49 mass %, more preferably 0 to 5 mass %, further preferably 0 to 1 mass %, particularly preferably 0 mass %, based on the total mass of the solid component.

The contents of the acid generating agent according to the present embodiment, the acid diffusion controlling agent (E) and such other component (F) (the acid generating agent according to the present embodiment/acid diffusion controlling agent (E)/other component (F)) in the composition for optical component formation of the present embodiment are preferably 10 to 90/1 to 30/0 to 10, as expressed by mass % on the solid content basis. The content ratio among the respective components is selected from such various ranges so that the sum of the components is 10 mass %. The above content ratio allows performances such as sensitivity, resolution, and developability to be further excellent.

The method for preparing the composition for optical component formation of the present embodiment is not particularly limited, and examples thereof include a method including dissolving the respective components in the solvent in use to thereby provide a uniform solution, and thereafter, if necessary, filtering the solution by, for example, a filter having a pore size of about 0.2 μm.

The composition for optical component formation of the present embodiment can include other resin as long as the objects of the present invention are not impaired. Such other resin is not particularly limited, and examples thereof include a novolac resin, polyvinylphenols, polyacrylic acid, polyvinylalcohol, a styrene-maleic anhydride resin, and a polymer containing acrylic acid, vinylalcohol or vinylphenol as a monomer unit, or any derivative thereof. The content of the resin is not particularly limited, and appropriately regulated depending on the type of the acid generating agent according to the present embodiment, here used.

The cured product of the present embodiment is obtained by curing of the composition for optical component formation, and can be used for various resins. The cured product can be used as a highly versatile material imparting various characteristics such as a high melting point, a high refractive index and high transparency in various applications. The cured product can be obtained by subjecting the composition to a known method depending on each compositional profile, such as light irradiation or heating.

The cured product can be used for various synthetic resins such as an epoxy resin, a polycarbonate resin and an acrylic resin, and furthermore optical components such as a lens and an optical sheet, by means of functionality.

### EXAMPLES

Hereinafter, the present embodiment will be further specifically described with reference to Examples. However, the present invention is not limited to these Examples.

### Example 1

(Synthesis of BEPMS)

The following BEPMS was synthesized as shown in the following formula.

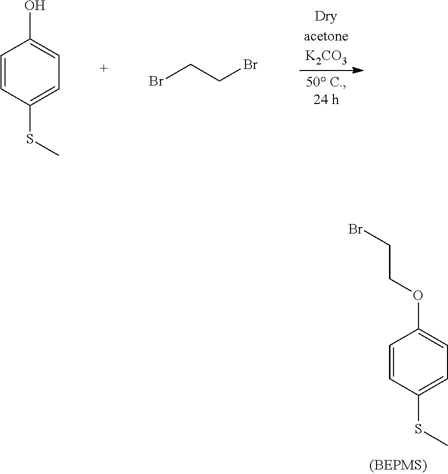

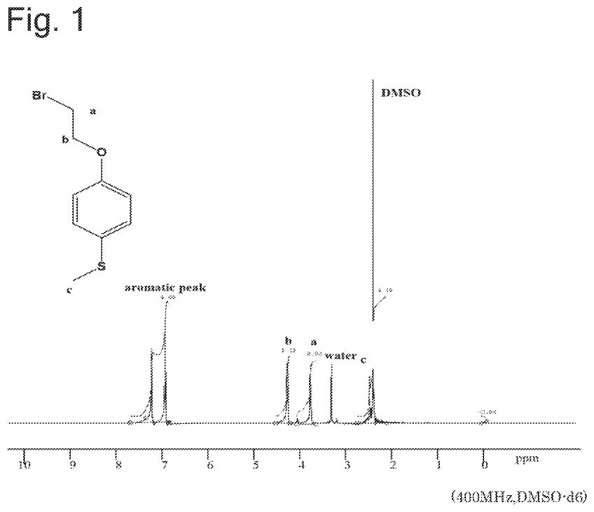

Specifically, BEPMS was synthesized by the following method. In a 200-ml eggplant flask, 4-methylthiophenol (22 mmol: 3.120 g) and potassium carbonate (85 mmol; 11.71 g) were dissolved in acetone (75 ml), and stirred under nitrogen at 0° C. for 15 minutes. Thereafter, dibromoethane (69 mmol: 12.90 g) was dropped, and the resultant was allowed to react at 50° C. for 24 hours. The resulting substance was subjected to membrane filtration, and the filtrate was applied to an evaporator for removal of the solvent, thereby providing a white solid (BEPMS). NMR and IR were used for structural analysis, and TLC measurement and melting point measurement were also performed. Thereafter, the product was purified by silica gel column chromatography with chloroform as a developing solvent. IR and NMR were used for structural analysis, and melting point measurement was also performed. The melting point was 64 to 66° C., the amount of yield was 1.43 g and the yield was 27.6%. A ¹H-NMR spectrum of the BEPMS is illustrated in FIG. 1.

(Synthesis of MTP-BEPMS)

The following MTP-BEPMS was synthesized as shown in the following formula.

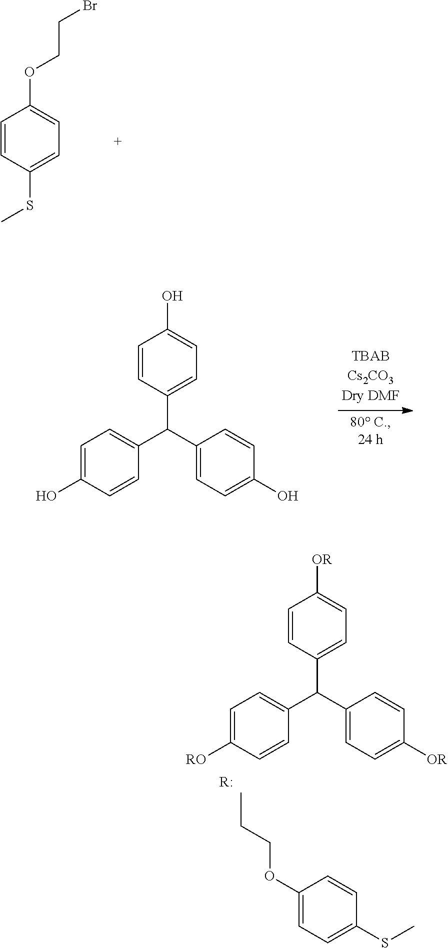

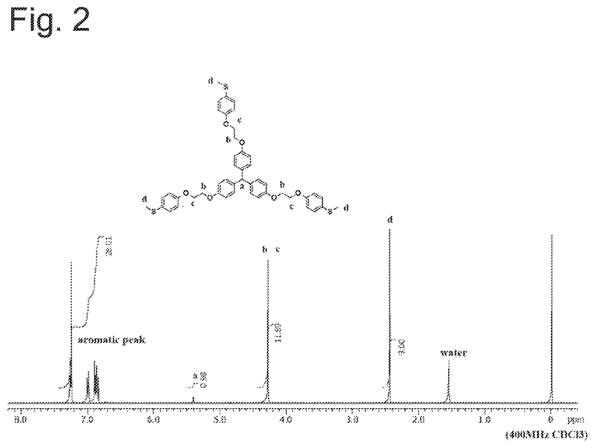

Specifically, MTP-BEPMS was synthesized by the following method. In a test tube, 4,4′,4″-trihydroxy-triphenylmethane (MTP) (0.5 mmol: 0.1461 g), cesium carbonate (2.0 mmol: 0.651 g), and TBAB (0.2 mmol: 0.0644) as a phase-transfer catalyst were dissolved in DMF (5 ml), and the resultant was stirred at 80° C. for 30 minutes. Thereafter, the BEPMS (2.0 mmol: 0.493 g) dissolved in DMF (2 ml) was dropped, and the resultant was allowed to react at 80° C. for 24 hours. The resulting substance was re-precipitated by 1 N HCl and subjected to Kiriyama's filtration, thereby providing a solid, and the solid was dissolved in chloroform, and re-precipitated by hexane. The resulting substance was subjected to membrane filtration, and thus an orange solid (MTP-BEPMS) was purified. NMR and IR were used for structural analysis, and melting point measurement was also performed. The amount of yield was 0.336 g, the yield was 85%, and the melting point was 112 to 113° C. A ¹H-NMR spectrum of the MTP-BEPMS is illustrated in FIG. 2.

(Synthesis of MTP-BEPMS Ion Compound)

The following MTP-BEPMS ion compound was synthesized as shown in the following formula.

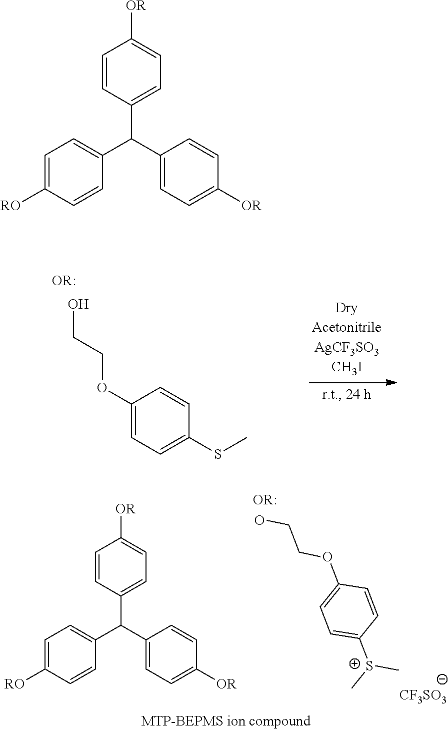

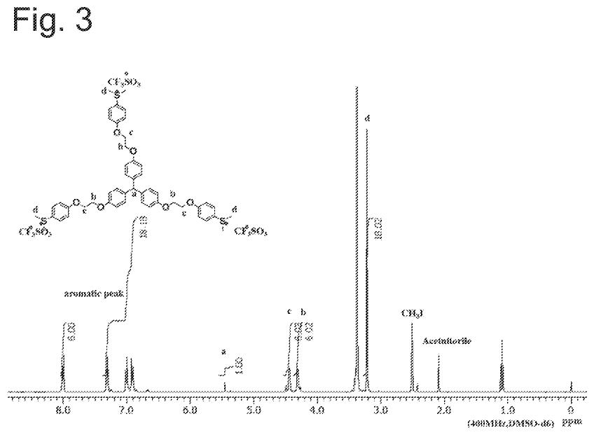

Specifically, an MTP-BEPMS ion compound was synthesized by the following method. The MTP-BEPMS (0.1 mmol: 0.0791 g) and AgCF₃SO₃BEPMS (0.4 mmol: 0.1027 g) were added to a 20-ml eggplant flask, the content was subjected to degassing and purging with nitrogen, thereafter iodomethane (0.4 mmol: 0.025 ml) and acetonitrile (5 ml) as a solvent were added thereto, and the resultant was allowed to react at ordinary temperature in a light-shielding condition for 24 hours. The resulting substance was subjected to membrane filtration, and the filtrate was applied to an evaporator for removal of the solvent, thereby providing a brown viscous solid. Thereafter, the solid was dissolved in acetone and re-precipitated by diethyl ether, and thus a brown viscous solid (MTP-BEPMS ion compound) was purified. NMR and IR were used for structural analysis. A ¹H-NMR spectrum of the MTP-BEPMS ion compound is illustrated in FIG. 3.

### Examples 2 to 7

Each ion compound shown in Table 1 was obtained by the same synthesis, in which each compound shown in Table 1 was used instead of 4,4′,4″-trihydroxy-triphenylmethane (MTP) used in Example 1. XBisN-1 here used was obtained in the same manner as in Synthesis Example 15 of International Publication No. WO2013/024778. BiF-1 here used was obtained in the same manner as in Synthesis Example 1 of International Publication No. WO2015/137485. NF71A7 here used was obtained in the same manner as the production of polyphenol (B) in International Publication No. WO2019/151403.

TABLE 1

| | Compound used for | |
|---|---|---|
| | synthesis | ion compound obtained |
| Example 1 | MTP | MTP-BEPMS ion compound |
| Example 2 | Bis-A | Bis-A-BEPMS ion compound |
| Example 3 | Bis-AF | Bis-AF-BEPMS ion compound |
| Example 4 | ODP | ODP-BEPMS ion compound |
| Example 5 | XBisN-1 | XBisN-1 -BEPMS ion compound |
| Example 6 | BiF-1 | BiF-1-BEPMS ion compound |
| Example 7 | NF71A7 | NF71A7-BEPMS ion compound |

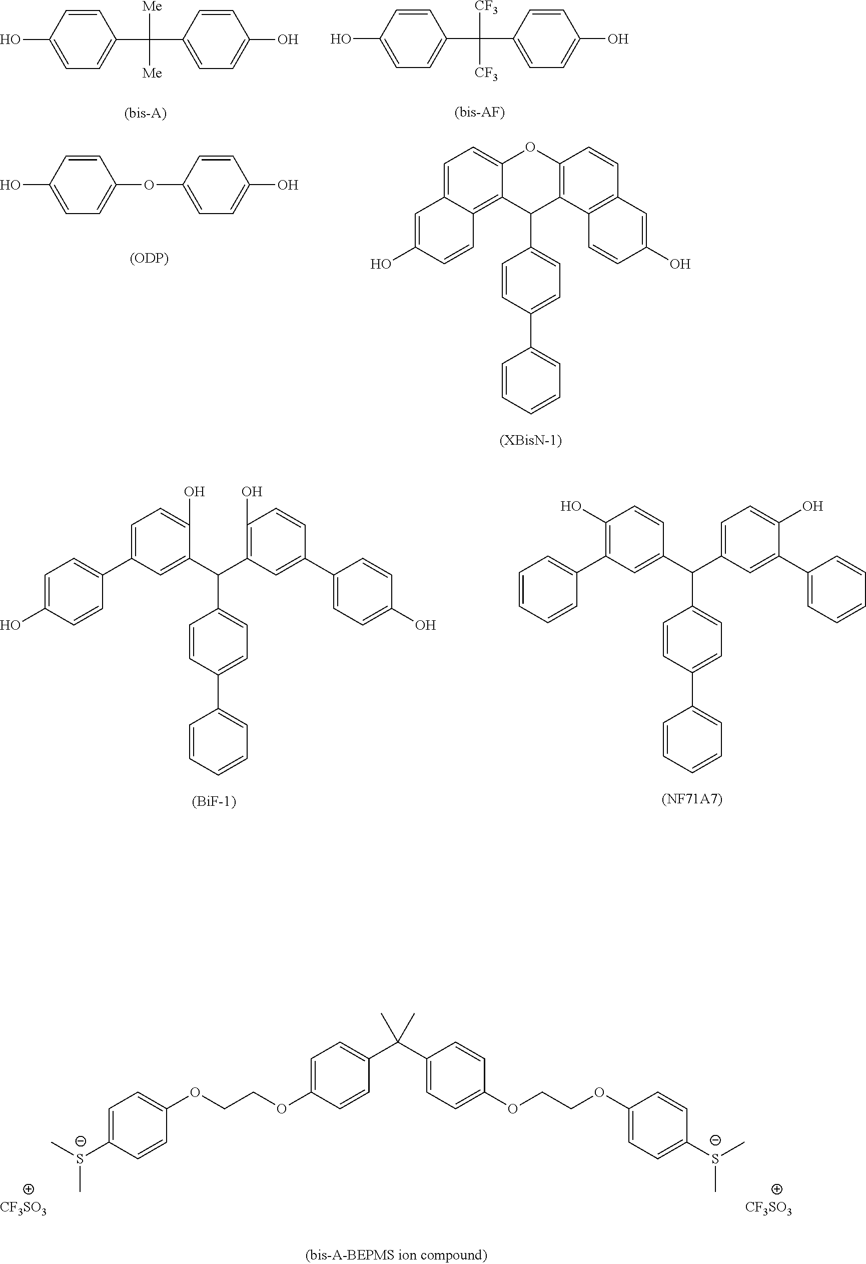

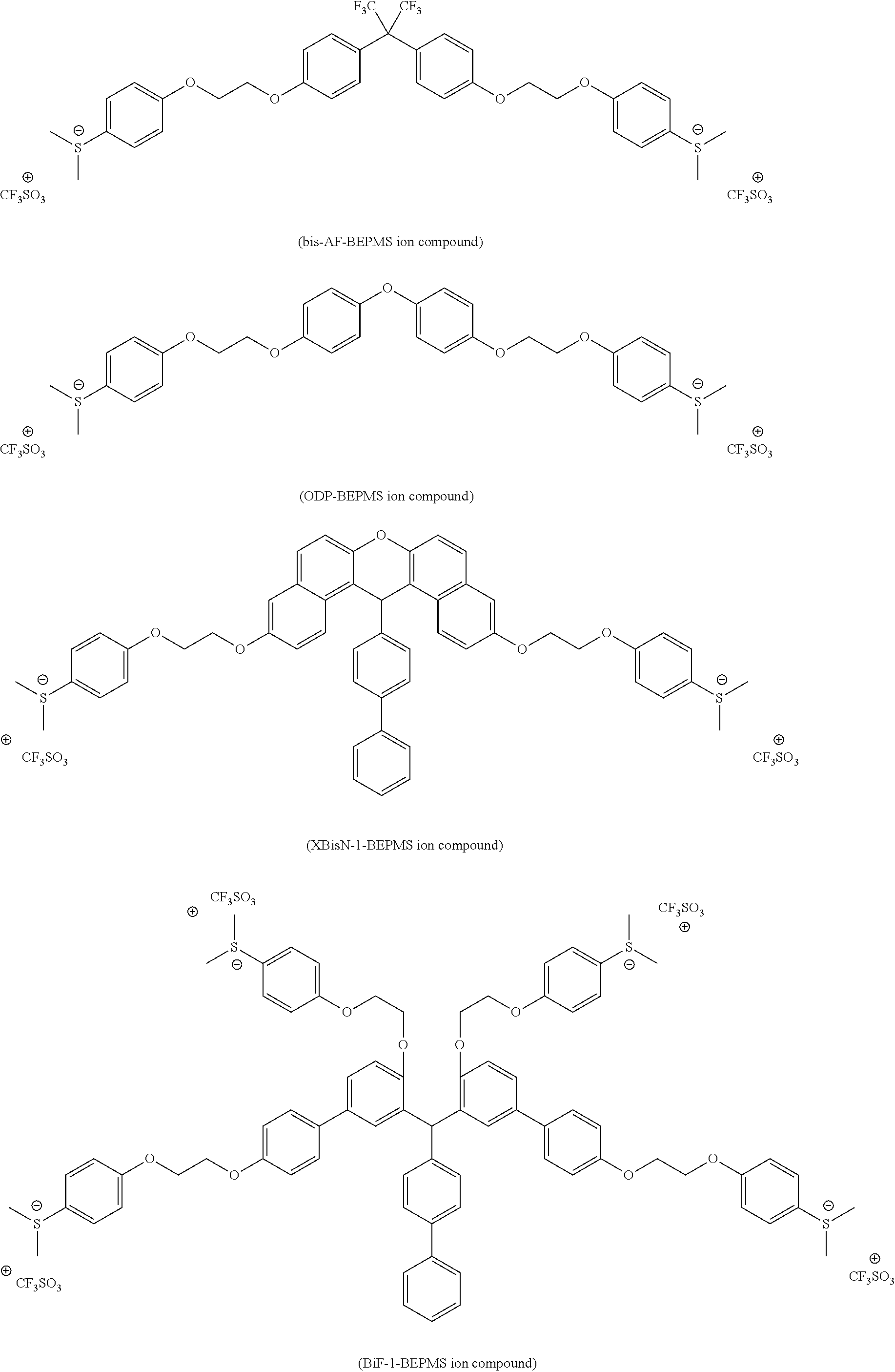

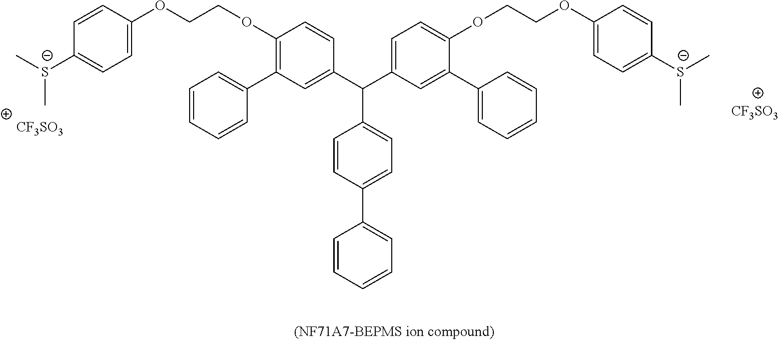

### Example 81

(Synthesis of BHPMS)

The following BHPMS was synthesized as shown in the following formula.

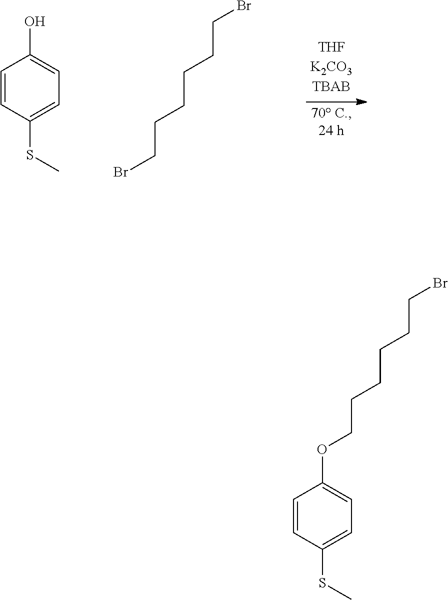

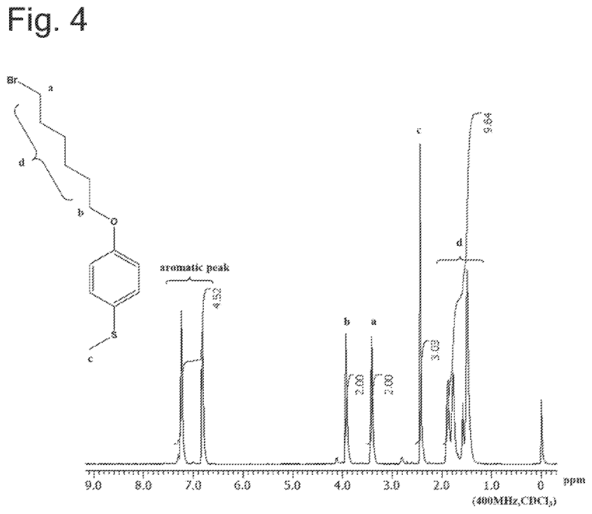

Specifically, BHPMS was synthesized by the following method. In a 100-ml eggplant flask, 4-methylthiophenol (7 mmol: 0.98 g) and potassium carbonate (7 mmol: 0.96 g) were dissolved in THF (30 ml), and stirred under nitrogen with reflex for 2 hours. Thereafter, 1,6 dibromohexane (35 mmol: 8.53 g) was added thereto, and the resultant was allowed to react at 70° C. for 24 hours. The resulting substance was extracted with hydrochloric acid and chloroform, and an organic layer was concentrated by an evaporator. Thereafter, a white solid (BHPMS) was obtained by re-precipitation with methanol as a poor solvent. Thereafter, the product was purified by silica gel column chromatography. IR and NMR were used for structural analysis, and melting point measurement was also performed. The melting point was 71 to 72° C., the amount of yield was 0.7 g and the yield was 33%. A ¹H-NMR spectrum of BHPMS is illustrated in FIG. 4.

(Synthesis of MTP-BHPMS)

The following MTP-BHPMS was synthesized as shown in the following formula.

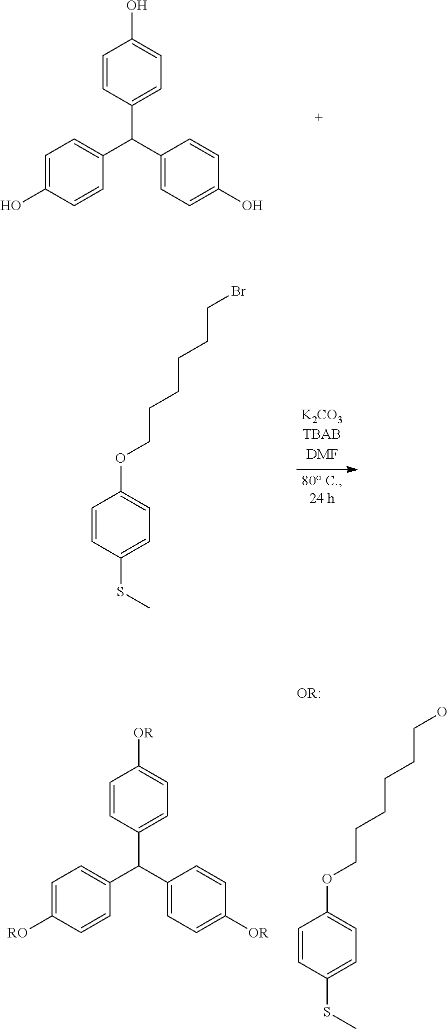

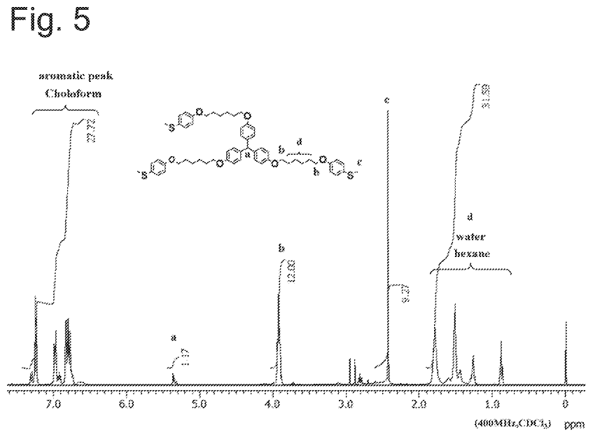

Specifically, MTP-BHPMS was synthesized by the following method. In a 50-mL eggplant flask, 4,4′,4″-trihydroxy-triphenylmethane (MTP) (0.5 mmol: 0.1461 g), potassium carbonate (2.0 mmol: 0.651 g), and TBAB (0.2 mmol: 0.0644) as a phase-transfer catalyst were dissolved in DMF (7 ml), and the resultant was stirred at 80° C. for 30 minutes. Thereafter, the BHPMS (2.0 mmol: 0.493 g) dissolved in DMF (3 ml) was dropped, and the resultant was allowed to react at 80° C. for 24 hours. The resulting substance was re-precipitated by 1 N HCl and subjected to Kiriyama's filtration, thereby providing a solid, and the solid was dissolved in chloroform, and re-precipitated by hexane. The resulting substance was recovered by decantation, thereby providing a red solid (MTP-BHPMS). NMR and IR were used for structural analysis. The amount of yield was 0.85 g and the yield was 88%. A ¹H-NMR spectrum of the MTP-BHPMS is illustrated in FIG. 5.

(Synthesis of MTP-BHPMS Ion Compound)

The following MTP-BHPMS ion compound was synthesized as shown in the following formula.

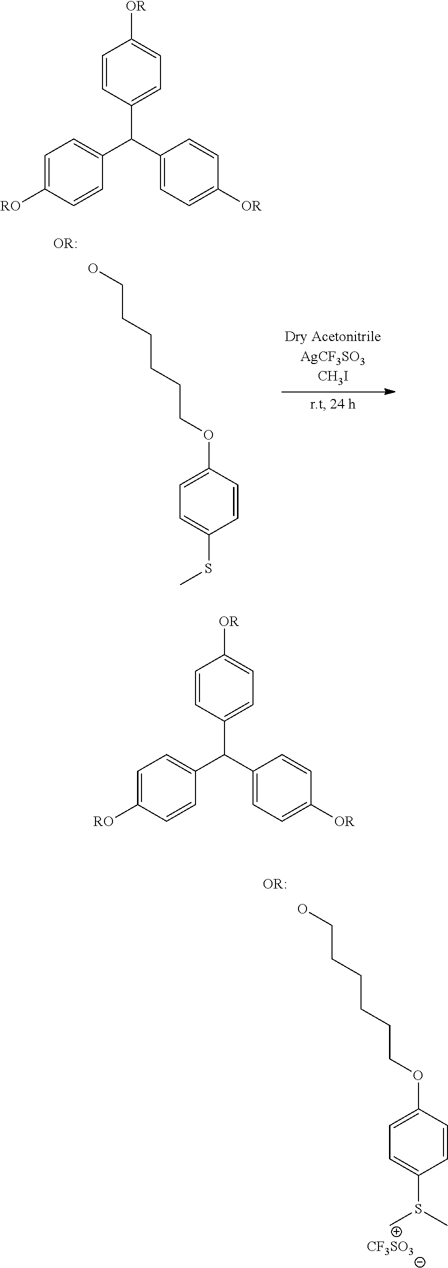

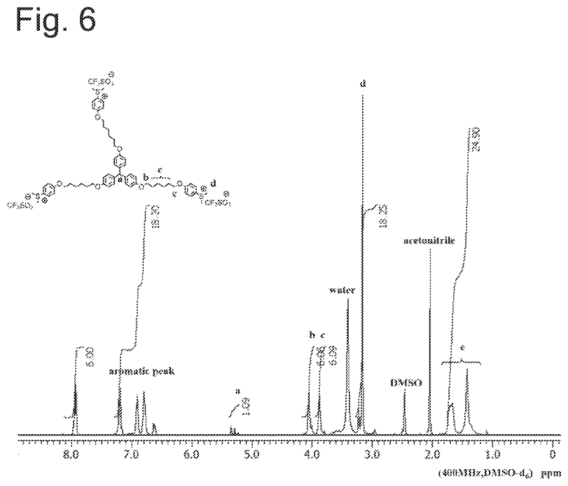

Specifically, an MTP-BHPMS ion compound was synthesized by the following method. The MTP-BHPMS (0.83 mmol: 0.80 g) and AgCF₃SO₃ (3 mmol: 0.77 g) were added to a 20-ml eggplant flask, the content was subjected to degassing and purging with nitrogen, thereafter iodomethane (3 mmol: 0.186 ml) and acetonitrile (5 ml) as a solvent were added thereto, and the resultant was allowed to react at ordinary temperature in a light-shielding condition for 24 hours. The resulting substance was subjected to membrane filtration, and the filtrate was applied to an evaporator for removal of the solvent, thereby providing a brown viscous solid. Thereafter, the solid was dissolved in acetone and re-precipitated by diethyl ether, and thus a red viscous solid (MTP-BHPMS ion compound) was purified. NMR and IR were used for structural analysis. A ¹H-NMR spectrum of the MTP-BHPMS ion compound is illustrated in FIG. 6.

[Evaluation of Heat Resistance]

The initial thermal decomposition temperature of each of the ion compounds obtained in Examples 1 to 8 was measured with a thermogravimetric apparatus (TGA). The measurement results are shown in Table 2.

TABLE 2

| | | Initial thermal |
|---|---|---|
| | | decomposition |
| | ion compound | temperature (° C.) |
| Example 1 | MTP-BEPMS ion compound | 280 |
| Example 2 | Bis-A-BEPMS ion compound | 190 |
| Example 3 | Bis-AF-BEPMS ion compound | 239 |
| Example 4 | ODP-BEPMS ion compound | 246 |
| Example 5 | XBisN-1-BEPI ion compound | 300 |
| Example 6 | BiF-1-BEPI ion compound | 250 |
| Example 7 | NF71A7-BEPI ion compound | 298 |
| Example 8 | MTP-BHPMS ion compound | 218 |

As shown in Table 2, all the ion compounds obtained in Examples 1 to 8 exhibited a high decomposition temperature. Therefore, it has been found that the compound according to the present embodiment has high heat resistance. The compound according to the present embodiment has heat resistance, can increase the baking temperature in film formation, and has the advantage of flattening. A film high in hardness can be obtained, and thus the compound can allow a pattern high in resolution to be maintained, when used as a resist or an underlayer film to form a pattern.

### Comparative Example 1

(Synthesis of AC-1)

AC-1 as a resin having a structure represented by the following formula was synthesized.

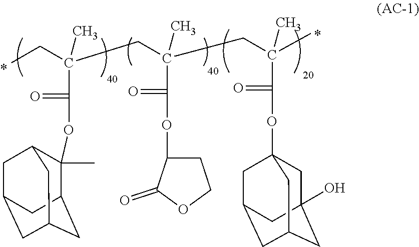

Specifically, AC-1 was synthesized by the following method. A reaction solution was obtained by dissolving 4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile in 80 mL of tetrahydrofuran. The reaction solution was subjected to polymerization under a nitrogen atmosphere for 22 hours, with the reaction temperature being retained at 63° C., and thereafter the reaction solution was dropped into 400 mL of n-hexane. The resulting resin was solidified and purified, and the resulting white powder was subjected to filtration, and then dried at 40° C. under reduced pressure overnight, to thereby obtain AC-1.

[Evaluation of Sensitivity]

Each of the ion compounds obtained in Examples 1 to 8 was dissolved in propylene glycol monomethyl ether, to thereby provide a 3% solution. The solution was dropped on a silicon wafer by use of a spin coater at 3300 rpm for 30 seconds. The resultant was baked at 90° C. for 60 seconds, to thereby obtain a thin film of 50 to 80 nm. After measurement of the film thickness, the film was irradiated with EUV by “EUV exposure apparatus (EUVES-7000)” manufactured by Litho Tech Japan Corporation, and dipped in ion-exchange water for 30 seconds to thereby perform development. The irradiance of EUV, at which the film thickness was 0, was defined as the sensitivity. The sensitivity was measured in the same manner, with respect to resin AC-1 obtained in Comparative Example 1, by dipping in a 2.38 mass % TMAH alkaline developing solution for 60 seconds to perform development, instead of dipping in ion-exchange water for 30 seconds. The results are shown in Table 3.

TABLE 3

| | | Sensitivity |
|---|---|---|
| | Ion compound | (mJ/cm²) |
| Example 1 | MTP-BEPMS ion compound | 18 |
| Example 2 | bis-A-BEPMS ion compound | 20 |
| Example 3 | bis-AF-BEPMS ion compound | 30 |
| Example 4 | ODP-BEPMS ion compound | 30 |
| Example 5 | XBisN-1-BEPMS ion compound | 25 |
| Example 6 | BiF-l-BEPMS ion compound | 30 |
| Example 7 | NF71A7-BEPMS ion compound | 30 |
| Example 8 | MTP-BHPMS ion compound | 25 |
| Comparative | AC-1 | 40 or more |
| Example 1 | | |

It has been found from the results in Table 3 that the compound according to the present embodiment is high in sensitivity and can be used as a resist high in sensitivity. The compound according to the present embodiment achieves high sensitivity without addition of any acid generating agent, thus requires no use of any chemical amplification mechanism with diffusion of an acid causing roughness, and allows a pattern high in resolution to be obtained when used as a resist.

[Evaluation of Heat Resistance in Case of Use as Acid Generating Agent]

A composition was prepared by compounding each of the ion compounds obtained in Examples 1 to 8, and di-tert-butyldiphenyliodonium nonafluoromethanesulfonate (DTDPI) manufactured by Midori Kagaku Co., Ltd., with XBisN-1 used as a raw material in Example 5, Nikalac MX270 (Nikalac) manufactured by Sanwa Chemical Co., Ltd., and propylene glycol monomethyl ether acetate (PGMEA), in amounts shown in Table 4. The unit of each numerical value in brackets in Table 4 is “parts by mass”.

TABLE 4

| | | | Crosslinking | |
|---|---|---|---|---|
| | Phenol | Acid generating agent | agent | Solvent |
| Example 1 | XBisN-1 | MTP-BEPMS | Nikalac | PGMEA |
| | (10) | ion compound (0.5) | (0.5) | (90) |
| Example 2 | XBisN-1 | bis-A-BEPMS | Nikalac | PGMEA |
| | (10) | ion compound (0.5) | (0.5) | (90) |
| Example 3 | XBisN-1 | bis-AF-BEPMS | Nikalac | PGMEA |
| | (10) | ion compound (0.5) | (0.5) | (90) |
| Example 4 | XBisN-1 | ODP-BEPMS | Nikalac | PGMEA |
| | (10) | ion compound (0.5) | (0.5) | (90) |
| Example 5 | XBisN-1 | XBisN-1-BEPMS | Nikalac | PGMEA |
| | (10) | ion compound (0.5) | (0.5) | (90) |
| Example 6 | XBisN-1 | BiF-1 -BEPMS | Nikalac | PGMEA |
| | (10) | ion compound (0.5) | (0.5) | (90) |
| Example 7 | XBisN-1 | NF71A7-BEPMS | Nikalac | PGMEA |
| | (10) | ion compound (0.5) | (0.5) | (90) |
| Example 8 | XBisN-1 | MTP-BHPMS | Nikalac | PGMEA |
| | (10) | ion compound (0.5) | (0.5) | (90) |
| Comparative | XBisN-1 | None | None | PGMEA |
| Example 2 | (10) | | | (90) |
| Comparative | XBisN-1 | DTDPI | Nikalac | PGMEA |
| Example 3 | (10) | (0.5) | (0.5) | (90) |

A silicon wafer where the film thickness was 300 nm was coated with the composition by spin coating, and baked at 150° C. for 60 seconds, and thus a film having a thickness of 100 nm was formed. Further baking was made at 400° C. for 60 seconds, and the rate of reduction in film thickness was measured. A rate of reduction in film thickness of less than 40% was rated as “A”, a rate of 40% or more and less than 60% was rated as “B”, and a rate of 60% or more was rated as “C”. The rating results are shown in Table 5.

TABLE 5

| | Rating of reduction |
|---|---|
| | in film thickness |
| Example 1 | A |
| Example 2 | A |
| Example 3 | A |
| Example 4 | A |
| Example 5 | A |
| Example 6 | A |
| Example 7 | A |
| Example 8 | A |
| Comparative | C |
| Example 2 | |
| Comparative | B |
| Example 3 | |

It has been found from Table 5 that the compound according to the present embodiment, when used as an acid generating agent, can form a film high in heat resistance.

From the foregoing, the compound according to the present embodiment can be suitably used for a resist film, an underlayer film, and an optical article.

1. A compound represented by the following formula (P-0): 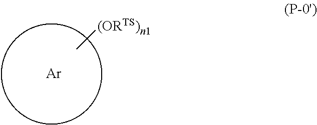
wherein Ar is a group having an aryl group having 6 to 60 carbon atoms, each OR^{TS} is independently a hydroxy group, a group represented by the following formula (TS-0), or a group represented by the following formula (TS-1), and n′ is an integer of 1 to 20, provided that at least one OR^{TS} is a group represented by the following formula (TS-0) or a group represented by the following formula (TS-1): 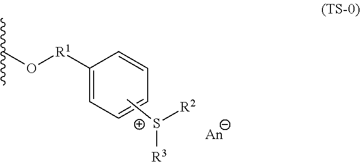
wherein R¹ is a single bond or a divalent group having 1 to 30 carbon atoms and optionally having a substituent, R² is an alkyl group having 1 to 10 carbon atoms and optionally having a substituent or an aryl group having 6 to 10 carbon atoms and optionally having a substituent, R³ is an alkyl group having 1 to 10 carbon atoms and optionally having a substituent or an aryl group having 6 to 10 carbon atoms and optionally having a substituent, and An⁻ is an anion containing fluorine or iodine; 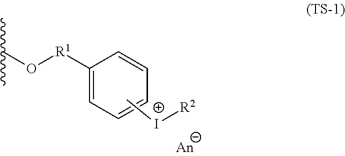
wherein R¹, R³ and An⁻ have the same meanings as in formula (TS-0).

2. The compound according to claim 1, wherein, in formula (TS-0) and formula (TS-1), R³ is an alkyl group having 1 to 10 carbon atoms and optionally having a substituent, and An⁻ is R⁴SO₃⁻, wherein R⁴ is a monovalent group containing fluorine or iodine, having 1 to 9 carbon atoms and optionally having a substituent.

3. The compound according to claim 1, wherein R¹ in formula (TS-0) and formula (TS-1) is a divalent group having 2 to 6 carbon atoms and optionally having a substituent.

4. The compound according to claim 1, wherein R² in formula (TS-0) is a methyl group or an ethyl group.

5. The compound according to claim 4, wherein R² in formula (TS-0) is a methyl group.

6. The compound according to claim 1, wherein, in formula (TS-0) and formula (TS-1), R³ is a methyl group and An⁻ is CF₃SO₃⁻.

7. The compound according to claim 1, wherein the compound is a compound represented by the following formula (P-0 Å): 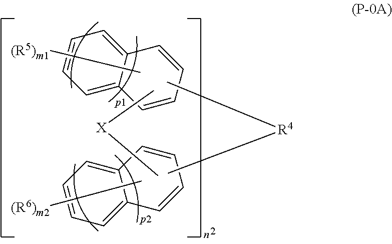
wherein each X is independently an oxygen atom, a sulfur atom or non-crosslinking, R⁴ is a single bond or a 2n-valent group having 1 to 30 carbon atoms and optionally having a substituent, R⁵ and R⁶ are each independently a halogen atom, a straight alkyl group having 1 to 30 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a cyano group, a nitro group, an amino group, a carboxylic acid group, a thiol group, a hydroxy group, the group represented by formula (TS-0), or the group represented by formula (TS-1), wherein the alkyl group, the aryl group, the alkenyl group, the alkynyl group, and the alkoxy group each optionally contain an ether bond, a ketone bond or an ester bond, m¹ and m² are each independently an integer of 0 to 7, p¹ and p² are each independently 0 or 1, and n² is an integer of 1 to 4, provided that at least one of m¹ and m² is an integer of 1 to 7 and formula (P-0A) contains at least one of the group represented by formula (TS-0) or the group represented by formula (TS-1) as R⁵ or R⁶.

8. The compound according to claim 1, wherein the compound is a compound represented by the following formula (P-0B): 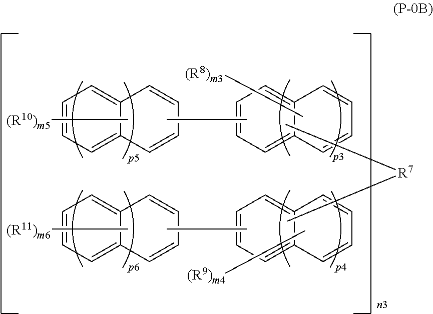
wherein R⁷ is a 2n-valent group having 1 to 30 carbon atoms, R⁸ to R¹¹ are each independently a halogen atom, a straight alkyl group having 1 to 10 carbon atoms and optionally having a substituent, a branched alkyl group having 3 to 30 carbon atoms and optionally having a substituent, a cyclic alkyl group having 3 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a thiol group, a cyano group, a nitro group, an amino group, a carboxylic acid group, a hydroxy group, the group represented by formula (TS-0), or the group represented by formula (TS-1), wherein the alkyl group, the aryl group, the alkenyl group, the alkynyl group, and the alkoxy group each optionally contain an ether bond, a ketone bond or an ester bond, m³ and m⁴ are each independently an integer of 0 to 8, m⁵ and m⁶ are each independently an integer of 0 to 9, p³ to p⁶ are each independently an integer of 0 to 2, and n³ is an integer of 1 to 4, provided that at least one of m³, m⁴, m⁵ and m⁶ is an integer of 1 or more and formula (P-0B) contains at least one of the group represented by formula (TS-0) or the group represented by formula (TS-1), as R⁸, R⁹, R¹⁰ or R¹¹.

9. The compound according to claim 1, wherein the compound is a compound represented by the following formula (P-0C): 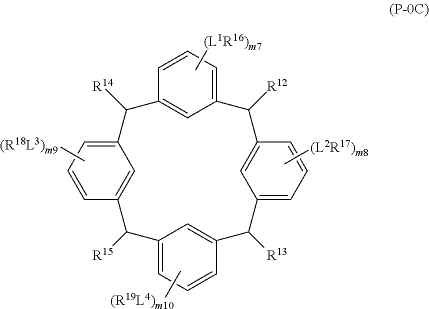
wherein L¹ to L⁴ are each independently a single bond, a straight alkylene group having 1 to 20 carbon atoms and optionally having a substituent, a branched alkylene group having 3 to 20 carbon atoms and optionally having a substituent, a cycloalkylene group having 3 to 20 carbon atoms and optionally having a substituent, an arylene group having 6 to 24 carbon atoms and optionally having a substituent, —O—, —OC(═O)—, —OC(═O)O—, —N(R²⁰)—C(═O)—, —N(R²⁰)—C(═O)O—, —S—, —SO—, or —SO₂—, R²⁰ is a hydrogen atom or an alkyl group having 1 to 10 carbon atoms and optionally having a substituent, R¹⁶ to R¹⁹ are each independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, the group represented by formula (TS-0), the group represented by formula (TS-1), a cyano group, a nitro group, a hydroxy group, a heterocyclic group, a halogen atom, a carboxyl group, an alkylsilyl group having 1 to 20 carbon atoms; a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid; or a hydrogen atom, R¹² to R¹⁵ are each independently an alkyl group having 2 to 20 carbon atoms, the group represented by formula (TS-0), the group represented by formula (TS-1), or a group represented by the following formula (P-0C-1): 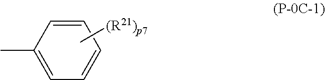
each R²¹ is independently an alkyl group having 1 to 20 carbon atoms and optionally having a substituent, a cycloalkyl group having 3 to 20 carbon atoms and optionally having a substituent, an aryl group having 6 to 20 carbon atoms and optionally having a substituent, an alkoxy group having 1 to 20 carbon atoms and optionally having a substituent, a cyano group, a nitro group, a heterocyclic group, a halogen atom, a carboxyl group, an alkylsilyl group having 1 to 20 carbon atoms; or a substituted methyl group having 2 to 20 carbon atoms, a 1-substituted ethyl group having 3 to 20 carbon atoms, a 1-substituted-n-propyl group having 4 to 20 carbon atoms, a 1-branched alkyl group having 3 to 20 carbon atoms, a silyl group having 1 to 20 carbon atoms, an acyl group having 2 to 20 carbon atoms, a 1-substituted alkoxyalkyl group having 2 to 20 carbon atoms, a cyclic ether group having 2 to 20 carbon atoms, an alkoxycarbonyl group having 2 to 20 carbon atoms, or an alkoxycarbonylalkyl group, each of which has the property of being dissociated by an acid, provided that at least one of R¹² to R¹⁹ is the group represented by formula (TS-0) or the group represented by formula (TS-1); m⁷ to m¹⁰ are each independently an integer of 1 to 4, and p⁷ is an integer of 0 to 5.

10. The compound according to claim 1, wherein the compound is a compound represented by the following formula (P-1): 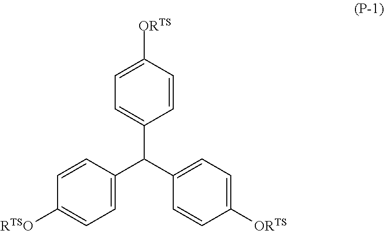
wherein OR^{TS} has the same meaning as in formula (P-0).

11. A composition comprising the compound according to claim 1.

12. The composition according to claim 11, further comprising a solvent.

13. The composition according to claim 11, further comprising an acid generating agent.

14. The composition according to claim 11, further comprising an acid crosslinking agent.

15. A resist film formed from the composition according to claim 11.

16. A method for forming a pattern, comprising
a film formation step of forming a film on a substrate by use of the composition according to claim 11,
an exposure step of exposing the film, and
a development step of developing the film exposed in the exposure step, to thereby form a pattern.

17. A method for producing the compound according to claim 1, comprising
a step of condensing a compound represented by the following formula (P-0′), and a compound represented by the following formula (TS-0′) or a compound represented by the following formula (TS-1′) to thereby obtain a condensate, and
a step of reacting the condensate, a salt having an anion containing fluorine or iodine, and an alkylating agent: 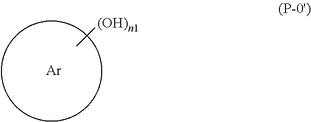
wherein Ar and n¹ have the same meanings as in formula (P-0); 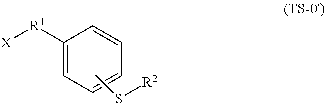
wherein X is a halogen atom, and R¹ and R² have the same meanings as in formula (TS-0); 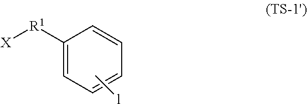
wherein X is a halogen atom and R¹ has the same meaning as in formula (TS-1).

18. An acid generating agent comprising the compound according to claim 1.

19. A composition comprising the acid generating agent according to claim 18.

20. The composition according to claim 19, further comprising a solvent.

21. The composition according to claim 19, further comprising an acid crosslinking agent.

22. The composition according to claim 19, wherein the composition is a composition for underlayer film formation for lithography.

23. The composition according to claim 22, further comprising a silicon-containing compound.

24. An underlayer film formed from the composition according to claim 22.

25. A method for forming a pattern, comprising
a step of forming a resist underlayer film by use of the composition according to claim 22,
a step of forming at least one photoresist layer on the resist underlayer film, and
a step of irradiating a predetermined region of the photoresist layer with radiation to thereby perform development.

26. The composition according to claim 19, wherein the composition is a composition for optical article formation.

27. An optical article formed from the composition according to claim 26.
